(19) United States
(12) Patent Application Publication
(10) Pub. No.: US 2008/0300222 A1
(43) Pub. Date: Dec. 4, 2008

(54) Phosphoantigen Salts Of Organic Bases And Methods For Their Crystallization

(75) Inventors: Gerard Coquerel, Boos (FR); Eric Aubin, Mont Saint Aignan (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Innate Pharma, Marseille (FR)

(21) Appl. No.: 12/088,477

(22) PCT Filed: Oct. 5, 2006
(86) PCT No.: PCT/EP06/67089

§ 371 Date: Mar. 28, 2008

Related U.S. Application Data
(60) Provisional application No. 60/724,308, filed on Oct. 6, 2005.

(51) Int. Cl. A61K 31/675; C07F 9/06; A61K 31/661; A61P 37/02

(52) U.S. Cl. 514/82; 546/23; 562/11; 514/106

(57) ABSTRACT

The invention provides novel phosphoantigen salts and novel crystalline phases of phosphoantigens salts, that the latter including non-solvated polymorphs, and solvates useful as pharmaceuticals. The invention also provides pharmaceutical compositions comprising, and processes for making, novel phosphoantigen crystalline phases. Methods of using such compositions for the treatment of disease, immunostimulatory or immune response modifying use are also provided. The invention also provides method for obtaining phosphoantigen crystals as well as highly pure phosphoantigen compositions.

### FIELD

The invention provides novel phosphoantigen salts and novel crystalline phases of phosphoantigens salts, that the latter including non-solvated polymorphs, and solvates useful as pharmaceuticals. The invention also provides pharmaceutical compositions comprising, and processes for making, novel phosphoantigen crystalline phases. Methods of using such compositions for the treatment of disease, immunostimulatory or immune response modifying use are also provided. The invention also provides method for obtaining phosphoantigen crystals as well as highly pure phosphoantigen compositions.

### BACKGROUND

A number of related phosphate containing compounds have been recently been proposed or developed as pharmaceutical agents. Certain compounds are phosphoesters or more generally phosphate-containing compounds, such as nucleotides (or polynucleotides), nucleotide analogs, for example 7-deaza-dGTP and 7-deaza-dATP, or bisphosphonates such as zoledronate (Novartis). Both nucleotides or nucleotide-related molecules as well as bisphosphonates have been found to have immunomodulatory activities. For example, Aldara® (3M, Minneapolis, USA) is being developed as an immune response modifier. Zoledronate, pamidronate and other bisphosphonates inhibit bone resorption and have been developed for osteoporosis, bone metastases, multiple myeloma and other disorders that are characterized by bone fragility, but were more recently investigated for their ability to activate gamma delta T cells (Kunzmann et al, (2000) Blood 96(2): 384-392). A number of other phosphate-containing compounds that comprise a pyrophosphate moiety have been proposed as immunomodulatory agents (Espinosa et al. (2001) Microbes and Infection (3): 645-654).

A number of these compounds have been used in research for various applications, while others are being developed for human therapeutic use. One biological property for which some of these compounds are desired is their immunostimulatory or immunomodulatory activity. Numerous phosphate containing compounds have been found to have the capacity to activate or stimulate Vγ9Vδ2 T cells, either directly or indirectly. For example, isopentenyl pyrophosphate (IPP) stimulates Vγ9Vδ2 T cells. More recently, the phosphoantigen called bromohydrin pyrophosphate (BrHPP) has entered clinical development (Phosphostim™, Innate Pharma S.A., France) for the treatment of humans in cancer indications. Several other compounds of the phosphoantigens are under investigation for future treatment of humans in a number of different therapeutic indications. Hintz M., Jomaa H., FEBS Letters (2001) 317; Giner J-L., Tetrahedron Letters 43 (2002) 5457; Amslinger S., J. Org. Chem., 67 (2002) 4590; Fox D. T., J. Org. Chem., 67 (2002) 5009; Wolff M., Tetrahedron Letters 43 (2002) 2555; Hecht S., Tetrahedron Letters 43 (2002) 8929; Espinosa et al. (J. Biol. Chem., 2001, Vol. 276, Issue 21, 18337-18344); U.S. Pat. Nos. 6,660,723, 5,639,653 and 6,624,151; European Patent No. 1109818B1, PCT patent publication nos. WO 95/20673, WO 00/12516, WO 00/12519, WO 03/050128, WO 02/083720 and WO 03/009855).

However, to date no method exists which permit these compounds to be obtained in highly pure form, which is desirable for compounds to be used in human therapy. Currently available methods such as preparative liquid chromatography generally involve expensive and time-consuming technologies and do not permit compositions substantially free of impurities and having a highly stable and non-hygroscopic character to be obtained. Stability and non-hygroscopicity are important criteria in the manufacturing of pharmaceutical products. Manufacturing of pharmaceutical products usually proceeds with preparation of batches of an API (active pharmaceutical ingredient) which must then be stored prior to filling of vials or more generally prior to use. High stability is also necessary for certain pharmaceutical forms such as tablets or capsules for example.

There is therefore a need in the art for compositions, particularly pharmaceutical compositions, of phosphoantigens which are of high purity, relatively non-hygroscopic, and highly stable and convenient manufacturing methods to obtain them.

### SUMMARY OF THE INVENTION

### General Crystalline Phases

The invention provides novel crystalline phases of phosphoantigens salts that include, non-solvated polymorphs and solvates useful as pharmaceuticals. The invention also provides pharmaceutical compositions comprising, and processes for making, these phosphoantigen crystalline. The invention also provides highly pure phosphoantigen compositions, including particularly pharmaceutical compositions comprising a phosphoantigen, generally as the API, in highly pure form. The invention also provides a novel process of obtaining highly pure phosphoantigen in a cost effective manner. Methods of using such compositions for the treatment or prevention of disease and most preferably for the direct or indirect activation of Vγ9Vδ2 T cells are also provided. In a preferred embodiment, the invention provides a crystalline phosphoantigen phase.

In one aspect the invention discloses a salt comprising the reaction product of phosphoantigen and an organic base, said salt is selected from the group consisting of:
(a) a crystalline phase of a phosphoantigen; a non-solvated polymorph of a salt of a phosphoantigen; and
(b) a solvate of a salt of a phosphoantigen.
   Preferably said organic base is (i) a natural alkaloids of quinquina, including but not limited to quinine, cinchonidine, cinchonine, and quinidine; (ii) a quinoline base, including but not limited to 8-hydroxy-quinoline and 5-chloro-8-hydroxy-quinoline; or (iii) a pharmaceutically acceptable base, including but not limited to benzathin, procain, N-methyl-D-glucamine, as well as basic and polar amino-acids, for example lysine and arginine.

Further, in an embodiment of the invention, numerous different crystalline phases of different phosphoantigen species and organic bases are characterized by powder X-ray diffraction patterns expressed in terms of 2-theta angles and relative intensities (even if preferential orientation can modify the intensities). In particular, the present invention provides quinine, cinchonidine, 8-hydroxyquinoline, and benzathin salts of phosphoantigens characterized by powder X-ray diffraction patterns expressed in terms of 2-theta angles.

The invention also provides pharmaceutical compositions comprising, and processes for making, these phosphoantigen crystalline phases. Methods of using such compositions for the treatment or prevention of disease and most preferably for modulation of immune cells, for example a γδT cell, a dendritic cell, a B cell, a T cell (e.g. CD8+ or CD4+), an NK.T cell, or a natural killer (NK) cell are also provided.

Compounds of the invention include, but are not limited to, crystalline phases, including non-solvated polymorphs and solvates of a phosphoantigen quinine, phosphoantigen cinchonidine, phosphoantigen cinchonine, phosphoantigen quinidine, phosphoantigen 8-hydroxy-quinoline, phosphoantigen 5-chloro-8-hydroxy-quinoline, phosphoantigen benzathin, phosphoantigen procain, phosphoantigen N-methyl-D-glucamine, phosphoantigen lysine, phosphoantigen arginine, phosphoantigen diethylamine, phosphoantigen ethylenediamine, phosphoantigen piperazine, phosphoantigen ethanolamine, phosphoantigen thiethanolamine, phosphoantigen betaine salts.

Pharmaceutical dosage forms of the invention comprise therapeutically or prophylactically effective amounts of soluble crystalline phases of phosphoantigen as disclosed herein.

### General Crystallization Methodology

Currently available methods such as preparative liquid chromatography generally involve expensive and long technologies. One object of the present invention is to provide a new process for obtaining highly pure phosphoantigens in a convenient and cost-effective manner. The invention discloses a method for obtaining, purifying, isolating, making or otherwise making or preparing a phosphoantigen or geometric isomer thereof, or a composition comprising the foregoing. Also provided are methods for preparing or making a medicament, storing an API, intermediate or formulated medicament, or using (e.g. administering, treating an individual) applicable to a range of phosphoantigen compounds, as has been demonstrated in the Examples using structurally different phosphoantigen compounds. The method generally comprises:

(1) acidification of a phosphoantigen,

(2) salification with an organic base, and

(3) isolating the crystals.

Preferred exemplary protocols are further provided below, although it will be appreciated that the person of skill in the art may adapt protocols in order to prepare phosphoantigen crystalline phases.

The invention also provides a method of further purifying phosphoantigens, particularly phosphoantigen salts, said method comprising the steps of
(a) crystallizing a phosphoantigen, preferably according to preceding steps (1) to (3); and
(b) recrystallizing the phosphoantigen to obtain a substantially or essentially pure phosphoantigen or phosphoantigen composition, and/or advantageously a phosphoantigen or phosphoantigen composition substantially or essentially free of impurities.
   Optionally said method further comprises step:
(c) formulating a drug product, preferably by adding a pharmaceutically acceptable organic base.

In a particularly preferred method and as further shown herein in the examples and in FIG. 22, a specific protocol that can be used to crystallize a wide range of phosphoantigens comprises the steps of:
(a) acidification of the phosphoantigen, preferably by adding an acidic cation exchange resin to the phosphoantigen in solution;
(b) salification with an organic base solution;
(c) evaporation of solvent (distillation)-solid state formation (either amorphous or semi-crystalline);
(d) dissolution of the solid of step (c) in a minimum amount of a suitable solvent;
(e) anti-solvent addition (first stage of crystallization—formation of a suspension);
   or (e)′: slow evaporation of solvent in a controlled atmosphere;
   or (e)″: cooling of the solution;
(f) maturation cycles on the suspension (==crystal growth−crystallinity improvement);
(g) crystallized solid isolation (preferably by filtration of the suspension);
(h) optional washings or slurries; and
(i) drying of the crystallized solid.

Following step (i), the crystallization process can be repeated as many times as desired in one or more recrystallization cycles, in which case steps (d) to (i) are repeated using the solid obtained in step (i) of the preceding crystallization cycle. This will serve to increase chemical purity. Optionally, the method can further comprise step (j) solid formulation.

Optionally, a phosphoantigen can be further purified from crystallized form, preferably by carrying out one or more, preferably at least 2, 3, 4 or 5 successive re-crystallizations (i.e., in addition to the initial crystallization) using the method described. A re-crystallization preferably comprises repeating steps (d) to (i) of the preceding paragraph using the solid obtained in step (i) of the preceding crystallization cycle.

Crystallization attempts of phosphoantigen inorganic salts (sodium, potassium) have been rarely successful. The resulting compounds are in almost all cases amorphous and moreover highly hygroscopic. By contrast, the inventors have surprisingly found out that the use of selected organic bases to prepare phosphoantigen organic salts promotes the isolation of stable crystalline phases. Depending on the organic base used and/or preferences of the user, the phosphoantigen salt obtained by the aforementioned crystallization method can be used as an intermediate in the production of an API or a medicament, and may for example be stored for further use. In other aspects, the phosphoantigen salt obtained by the aforementioned crystallization method can be used directly as a medicament, or in a medicament, generally as the API. For use of the phosphoantigen salt directly as a medicament, any of a number of pharmaceutically acceptable bases may be used. Optionally, the methods may comprise further steps of formulating a drug product from purified phosphoantigen compound with pharmaceutically acceptable organic bases. In a particularly preferred embodiment, a pharmaceutically acceptable organic base is used in the crystallization of the phosphoantigen such that the crystalline phase obtained can be used directly as a medicament; for this purpose preferred pharmaceutically acceptable organic bases include but are not limited to quinine, cinchonidine, cinchonine, quinidine, 8-hydroxy-quinoline, 5-chloro-8-hydroxy-quinoline, benzathin, procain, N-methyl-D-glucamine, lysine, arginine, diethylamine, ethylenediamine, piperazine, ethanolamine, thiethanolamine, betaine.

As demonstrated in the Examples, the methods described herein can also be advantageously used to obtain, isolate, purify or prepare a stereoisomer of a phosphoantigen, particularly a geometric isomer of a phosphoantigen. Using the present method, an enriched, or preferably substantially or essentially pure geometric isomer of a phosphoantigen can be obtained, such substantially or essentially pure geometric isomer of a phosphoantigen compound or a composition comprising also being within the scope of the present invention. Preferably, a “pure stereoisomer” or “pure geometric isomer” is a composition that comprises one stereoisomer or geometric isomer, respectively, of a compound and is substantially free of, essentially free of, or free of, other stereoisomers or geometric isomer of that compound. Preferably, a geometric isomer having one geometric form will be substantially free of the 5 opposite geometric isomer of the compound. A typical pure geometric isomer comprises greater than about 90%, 95%, 98%, 99% or 99.5% by weight of one geometric isomer of the compound and less than about 10%, 5%, 2%, 1% or 0.5% respectively by weight of another geometric isomer of the compound.

### Novel Crystalline Phases

The invention discloses several crystalline phosphoantigen phases, particularly a crystalline phosphoantigen anhydrate, a crystalline phosphoantigen hydrate, a crystalline phosphoantigen monohydrate, a crystalline phosphoantigen dihydrate, a crystalline phosphoantigen trihydrate. Preferably said crystalline phases are substantially or essentially pure and most preferably in isolated form. Encompassed also are compositions comprising at least two different crystalline phases, particularly a composition which is a mixture of two, three or more different crystalline phases. Also encompassed are solvates of any of said crystalline phases. Encompassed also are pharmaceutical formulations comprising the foregoing.

The invention discloses several crystalline phases of a phosphoantigen salt, particularly a crystalline phosphoantigen quinine salt, a phosphoantigen cinchonidine salt, a phosphoantigen 8-hydroxyquinoline salt and a phosphoantigen benzathine salt. Particularly preferred is a crystalline phase of a compound of Formulas I to III, preferably a quinine salt, cinchonidine salt, cinchonine salt, quinidine salt, 8-hydroxy-quinoline salt, 5-chloro-8-hydroxy-quinoline salt, benzathin salt, procain salt, N-methyl-D-glucamine salt, lysine salt, arginine salt, diethylamine salt, ethylenediamine salt, piperazine salt, ethanolamine salt, thiethanolamine salt, betaine salt. In other specific embodiments, the phosphoantigen is selected from the group consisting of BrHPP, IPP, HDMAPP, C-HDMAPP, N-HDMAPP and H-angelylPP. Most preferably the phase is a crystalline phosphoantigen of at least one of Phases A to O, as described below.

In the framework of the present invention, the expression “Formulas I to III”, designate all compounds derived from Formulas I to III: I, II, IIa, II, IIIa, IIIa1, IIIa2, IIIa3, A, B, IIIb, IIIb1, IIIb2, IIIb3, C, IIIc, IIIc1, IIIc2, IIIc3, D, E, F and G, as defined below.

Another embodiment of the invention encompasses a crystalline solvate of phosphoantigen, particularly a salt of a compound of Formulas I to III, particularly a crystalline solvate of a quinine salt, a crystalline solvate of a cinchonidine salt, a crystalline solvate of a 8-hydroxyquinoline salt and a crystalline solvate of a benzathin salt. Preferably said compound of Formulas I to III is selected from the group consisting of BrHPP, IPP, HDMAPP, C-HDMAPP, N-HDMAPP and H-angelylPP.

In further preferred specific embodiments, the invention encompasses a crystalline bisphosphonate, nucleotide or nucleotide analog, salt or solvate, particularly a crystalline quinine salt or solvate, a cinchonidine salt or solvate, an 8-hydroxyquinoline salt or solvate and a benzathine salt or solvate, of a bisphosphonate, nucleotide or nucleotide analog.

In brief, the general method provided herein can be used to prepare any of the above crystalline phases. Said process for preparing the crystalline phase comprises the steps of dissolving the phosphoantigen in an aqueous solution and acidification, preferably using acidic or strongly acidic cation exchange resin, precipitating the crystalline phase by adding the organic base, preferably quinine, cinchonidine, 8-hydroxyquinoline solvate or benzathine; and isolating the crystals, preferably by evaporating the solvent. The process may further comprise cooling the solution prior to isolating the crystals. This general method is applicable broadly to phosphoantigens and any structurally related compounds.

The crystalline phase of phosphoantigen may be a hydrate of any of various stoechiometries, for example ratios of molecules of phosphoantigen to molecules of water at 0.5:1, 1:1, 1:1.5, 1:2, 1:3, 1:4, etc. Also encompassed is a phosphoantigen anhydrate.

Yet another embodiment of the invention encompasses a pharmaceutical composition comprising at least one of the crystalline phases of phosphoantigen selected from the group consisting of phases A to O.

### High Purity Phosphoantigen Salts or Compositions Containing the Same

The present invention provides high purity phosphoantigen compounds as well as method for obtaining them. The invention also provides compositions useful as medicaments including particularly but not limited to pharmaceutical formulations. In preferred embodiments are dosage forms suitable for human administration. Exemplary doses for various compounds as well as ranges for the phosphoantigen class are further provided herein. Preferred doses comprise between about 0.01 mg and 10 g, more preferably between 0.1 mg and 10 g, between 0.1 mg and 1 g, between 1 mg and 1 g, between 10 mg and 1 g or between 10 mg and 100 mg of essentially pure or substantially pure phosphoantigen. The inventors also provide a process for purifying phosphoantigen to a high degree and that is easily scaled for commercial production; in one preferred aspect the invention provides a composition according to any of the embodiments herein, comprising at least 1 g, 2 g, 5 g, 10 g, 20 g, 50 g, 100 g, 250 g, 500 g or 1 kg of high purity, preferably substantially or essentially pure phosphoantigen compound, and most preferably non-hygoscopic. Preferably the phosphoantigen is a crystalline phase.

In a preferred embodiment, the method is used to manufacture a phosphoantigen having anionic purity that is at least 95%, 97%, 98%, 99%, 99.5%, 99.8% or 99.9%, as measured by HPAEC (expressed as relative area percent). Preferably the phosphoantigen also exhibits reduced levels of impurities compared to phosphoantigen compositions prepared by prior art methods, and preferably essentially or substantially free of impurities. Preferably the phosphoantigen is a crystalline phase.

Further encompassed by the invention are pharmaceutical formulations comprising a phosphoantigen having anionic purity that is at least 95%, 97%, 98%, 99%, 99.5%, 99.8% or 99.9%, as measured by HPAEC (expressed as relative area percent). Preferably the phosphoantigen is a crystalline phase.

Also encompassed by the invention are compositions comprising a phosphoantigen and an organic base, preferably wherein the base is selected from the group consisting of (i) a natural alkaloids of quinquina, including but not limited to quinine, cinchonidine, cinchonine, and quinidine; (ii) a quinoline base, including but not limited to 8-hydroxy-quinoline and 5-chloro-8-hydroxy-quinoline; and (iii) a pharmaceutically acceptable base, including but not limited to benzathin, procain, N-methyl-D-glucamine, diethylamine, ethylenediamine, piperazine, ethanolamine, thiethanolamine, betaine, as well as basic and polar amino-acids, for example lysine and arginine.

Preferably said pharmaceutical formulations further comprise a pharmaceutically acceptable carrier.

Further encompassed by the invention are non-hygroscopic phosphoantigen compositions. In preferred aspects the invention provides a phosphoantigen composition that is essentially or substantially non-hygroscopic, and preferably said phosphoantigen composition has anionic purity of at least 95%, 97%, 98%, 99%, 99.5%, 99.8% or 99.9%, as measured by HPAEC (expressed as relative area percent).

In preferred embodiments of any of the embodiments herein, the invention provides a substantially or essentially non-hygroscopic phosphoantigen salt or composition containing the same. The phosphoantigen salt and composition containing the same preferably remains stable for at least 3 months, even at room temperature (or ambient temperature, i.e., about 25° C.) and in contact with ambient atmosphere, whereas BrHPP sodium salt (amorphous compound) undergoes a 50% degradation for the same storage conditions (see Examples). Most preferably the phosphoantigen is a substantially or essentially pure phosphoantigen salt. Hygroscopicity can be measured by any suitable method, and the phosphoantigen is determined to be substantially non-hygroscopic if the compound falls within the range for low hygroscopicity or non-hygroscopicity for the particular method used. For example dynamic vapor sorption (DVS), gravimetric methods or water content dosage methods (e.g. Karl Fisher) may be used. DVS is a well known method for measuring hygroscopicity; an example of a DVS protocol is described in the Examples. In one example hygroscopicity is evaluated by the method of Callahan et al ((1982) Drug. Dev. Ind. Pharm. 8(3):355-369), the disclosure of which is incorporated herein by reference, by determination of the equilibrium moisture content of a compound after storage at various relative humidities. The classification system of Callahan et al. (1982) can be used so that compound falling within at least low hygroscopicity or non-hygroscopic classification by Callahan et al. are considered non-hygroscopic for the purposes of the invention.

In another example, hygroscopicity is measured by dynamic moisture sorption gravimetry (DMSG) using a controlled atmosphere microbalance at a temperature of 25° C. Samples are analyzed over a relative humidity range of from 0 to 90% in 3% steps. Each step is brought to equilibrium before moving to the next step, with equilibrium assessed as a weight change of less than 0.002 mg (0.02%) for five consecutive points at 1 point per 120 seconds. Using this measure of hygroscopicity, non-hygroscopic salts of the invention exhibit a water uptake of less than 10%, preferably less than 5%, preferably less than 4%, more preferably less than 3%, more preferably less than 2%, more preferably less than 1% by weight, at 80% relative humidity.

In further preferred aspects, the high purity phosphoantigen salts or compositions containing the same have increased stability in storage conditions. Insofar as stability for storage is dependent on the conditions used for storage as well as the particular crystalline form, the invention provides a phosphoantigen compound which is stable for at least 1 month, or more preferably 2, 3, 6, 9 or 12 months in a controlled environment, in particular at room temperature, preferably at constant temperature and/or constant humidity. Stability preferably refers to anionic purity; preferably a compound remains stable over a given period if its anionic purity does not decrease by more than 3%, 2% or more preferably 1%. For example temperature may be 4° C., 10° C., 20° C., 25° C., 30° C., 35° C., 40° C., 50° C., 60° C., 75° C. or 100° C., or greater; such a temperature can be used with a given humidity, for example any of 10%, 20%, 30%, 40%, 50%, 60%, 70%, 80% or 90% relative humidity. The inventors provide several crystalline phosphoantigen salts which can be stored for such extended periods at temperatures up to and including ambient temperatures in most geographic regions, e.g. up to 20° C., 25° C., 30° C. or 35° C., providing important quality, increased safety as well as cost and logistical advantages. Stability also permits the development of tablet and other pharmaceutical forms of phosphoantigen; the invention thereby provides novel pharmaceutical forms including tablets and capsules comprising phosphoantigens. In preferred aspects of these embodiments, such a phosphoantigen composition is a crystalline phosphoantigen quinine salt, a phosphoantigen cinchonidine salt, a phosphoantigen 8-hydroxyquinoline salt and a phosphoantigen benzathine salt. Particularly preferred is a crystalline phase of a compound of Formulas I to III, preferably a quinine salt, a cinchonidine salt, a cinchonine salt, a quinidine salt, a 8-hydroxy-quinoline salt, a 5-chloro-8-hydroxy-quinoline salt, a benzathin salt, a procain salt, a n-methyl-d-glucamine salt, a lysine salt and an arginine salt. In other specific embodiments, the phosphoantigen is selected from the group consisting of BrHPP, IPP, HDMAPP, C-HDMAPP, N-HDMAPP and H-angelylPP. Most preferably the phase is a crystalline phosphoantigen of at least one of phases A to O.

According to a particular embodiment, the invention provides a salt of formula III made by reacting an organic base selected in the group consisting of quinine, cinchonidine, cinchonine, quinidine, 8-hydroxy-quinoline, 5-chloro-8-hydroxy-quinoline, benzathin, procain, N-methyl-D-glucamine, diethylamine, ethylenediamine, piperazine, ethanolamine, thiethanolamine, betaine, lysine, arginine and any other basic and polar amino-acid, wherein the form is stable for a period of time of at least 3 months at ambient temperature.

According to another particular embodiment, the invention provides a salt of Formula A to G made by reacting an organic base selected in the group consisting of quinine, cinchonidine, cinchonine, quinidine, 8-hydroxy-quinoline, 5-chloro-8-hydroxy-quinoline, benzathin, procain, N-methyl-D-glucamine, diethylamine, ethylenediamine, piperazine, ethanolamine, thiethanolamine, betaine, lysine, arginine and other basic and polar amino-acids, wherein the form is stable for a period of time of at least 3 or preferably 6 months at ambient temperature.

Furthermore, the invention provides a salt of Formula A to G made by reacting an organic base selected in the group consisting of quinine, cinchonidine, cinchonine, quinidine, 8-hydroxy-quinoline, 5-chloro-8-hydroxy-quinoline, benzathin, procain, N-methyl-D-glucamine, diethylamine, ethylenediamine, piperazine, ethanolamine, thiethanolamine, betaine, lysine, arginine and other basic and polar amino-acids, wherein the form is substantially non-hygroscopic or the form has a purity of at least 99.5%.

According to another particular embodiment, the invention provides a salt of BrHPP or C-HMAPP made by reacting an organic base selected in the group consisting of quinine, cinchonidine, 8-hydroxy-quinoline, and benzathin, wherein the form is stable for a period of time of at least 3 months at ambient temperature.

In another preferred embodiment, the method is used to manufacture phosphoantigen using reduced levels of solvents compared to those used in prior art methods.

Another object of the present invention is to provide a method for purifying phosphoantigen comprising the use of an organic base. In a preferred aspect, the method comprises (1) acidification of a phosphoantigen, preferably a phosphoantigen according to Formulas I to III; (2) salification with an organic base; and (3) isolating the crystals; and optionally (4) recrystallizing the phosphoantigen and/or; (5) formulating a drug product, preferably by adding a pharmaceutically acceptable organic base. In a preferred embodiment, the method is used to produce phosphoantigen having anionic purity of at least 95%, 97%, 98%, 99%, 99.5%, 99.8% or 99.9% (area percent based on HPAEC analysis) or that is substantially or essentially free of impurities. Preferably the high purity phosphoantigens or phosphoantigen compositions are substantially or essentially free of impurities, particularly inorganic phosphate, and/or pyrophosphate impurities. Pyrophosphate impurities are for example pyrophosphate-containing compound other than the pyrophosphate compounds sought, for example as will commonly remain in small amounts following synthesis. Most preferably the high purity phosphoantigens or phosphoantigen compositions comprise less than 3%, 2.5%, 2%, 1.5%, 1% or 0.5% (area percent based on HPAEC analysis) of inorganic phosphate and/or pyrophosphate impurities. Further specific examples of compounds are described further herein; for example the invention demonstrates that phosphoantigen compositions comprising less than 3%, 2.5%, 2%, 1.5%, 1% or 0.5% (area percent based on HPAEC analysis) of inorganic phosphate and/or inorganic pyrophosphate can be obtained. With respect to the latter example, the inventors demonstrate in FIGS. 17a and 17b (HPAEC profiles) that the phosphoantigens referred to as IPP and C-HDMAPP can be obtained substantially free of inorganic phosphate and pyrosphosphates.

### DESCRIPTION OF THE FIGURES

FIG. 1A shows the XRPD pattern for the crystalline Phase A: BrHPP Quinine salts, crystalline (n,p)-Mix-I.

FIG. 1B shows a thermogravimetric analysis of the crystalline BrHPP quinine salts (n,p)-Mix-I.

FIG. 2 shows the XRPD pattern for the crystalline Phase B: BrHPP Quinine salts, crystalline (n,p)-Mix-II.

FIG. 3 shows the XRPD pattern for the crystalline Phase C: BrHPP Quinine salts, crystalline (n,p)-Mix-III.

FIG. 4 shows the XRPD pattern for the crystalline BrHPP cinchonidine salts in the amorphous state.

FIG. 5 shows the XRPD pattern for the crystalline Phase D: BrHPP cinchonidine salts, crystalline (n,p)-Mix-I.

FIG. 6 shows the XRPD pattern for the crystalline Phase E: BrHPP cinchonidine salts, crystalline (n,p)-Mix-II.

FIG. 7 shows the XRPD pattern for the crystalline Phase F: BrHPP 8-hydroxyquinoline salts, crystalline Phase-I.

FIG. 8 shows the XRPD pattern for the crystalline Phase G: BrHPP benzathin salt (Rac)-Mix-I.

FIG. 9 shows the XRPD pattern for the crystalline Phase N: BrHPP benzathin salt (Rac)-Mix-II.

FIG. 10 shows the XRPD pattern for the crystalline Phase O: BrHPP benzathin salt (Rac)-Mix-III.

FIG. 11 shows the XRPD pattern for the (E) geometric isomer obtained in Example 5 for C-HDMAPP; shown is the XPRD patter for the crystalline Phase H: (E)-C-HDMAPP quinine salts, crystalline (E)-Phase-I.

FIG. 12 shows the XRPD pattern for the (E) geometric isomer obtained in Example 5 for C-HDMAPP; shown is the XRPD pattern for the crystalline Phase I: (E)-C-HDMAPP quinine salts, crystalline (E)-Phase-II.

FIG. 13 shows the XRPD pattern for the crystalline Phase J: (E)-C-HDMAPP benzathin salt, crystalline (E)-Phase-I.

FIG. 14 shows the XRPD pattern for the (E) geometric isomer obtained in Example 6 for C-HDMAPP; shown is the XRPD pattern for the crystalline Phase K: (E)-C-HDMAPP benzathin salt, crystalline (E)-Phase-II.

FIG. 15 shows the XRPD pattern for the crystalline Phase L: IPP quinine salt, crystalline Phase-I.

FIG. 16 shows the XRPD pattern for the crystalline Phase M: IPP quinine salt, crystalline Phase-II.

FIG. 17a is an example of an HPAEC profile for determining chemical purity (anionic purity) of a phosphoantigen salt (IPP Quinine salt, crystalline phase III) obtained according to the methods of the invention.

FIG. 17b is an example of an HPAEC profile for determining chemical purity (anionic purity) of a phosphoantigen salt (C-HDMAPP benzathin salt, crystalline (E)-phase II) obtained according to the methods of the invention.

FIG. 18 shows a DSC/TG analysis of amorphous BrHPP sodium salt.

FIG. 19 shows a DSC analysis of the (non solvated) crystalline phase prepared according to example 4 (BrHPP benzathin salts, crystalline (Rac)-Mix-I)), revealing a high thermal stability profile since the fusion-degradation event only occurs above 146° C.

FIG. 20 show DVS analysis of BrHPP sodium salt: FIG. 20A represents the sample mass variation and experimental R.H. versus time and FIG. 20B represents sorption and desorption isotherms at 20° C.

FIG. 21 show DVS analysis of BrHPP benzathin salt (Rac)-Mix-I: FIG. 21A represents the sample mass variation and experimental R.H. versus time and FIG. 21B represents sorption and desorption isotherms at 22° C.

FIG. 22 is a flow diagram of the general overview of the process for crystallization of phosphoantigens.

### DETAILED DESCRIPTION

Unless otherwise defined, all technical and scientific terms used herein have the meaning as commonly understood by one of ordinary skill in the art to which this invention belongs. The practice of the present invention employs, unless otherwise indicated, conventional techniques of chemistry, biochemistry and microbiology and basic terminology used therein. The given percentages (%) are expressed by weight unless otherwise specified.

The term “isolated” refers to a compound or product that is refers to a compound which represents at least 30%, more preferably at least 50%, 60% or 70%, and most preferably at least 80%, 90%, 95% or 98% of the compound present in the mixture.

A phosphoantigen or phosphoantigen composition is “substantially pure” when at least 98% of a sample is the particular phosphoantigen. Preferably, the phosphoantigen is “substantially pure” when at least 99% of a sample is the particular phosphoantigen.

A phosphoantigen or phosphoantigen composition is “essentially pure” when at least 99.5% of a sample is the particular phosphoantigen. Preferably, the phosphoantigen is “essentially pure” when at least 99.9% of a sample is the particular phosphoantigen.

A phosphoantigen or phosphoantigen composition is “substantially free” of another compound when the other compound(s) are present in an amount that is no more than 1% of the amount of the phosphoantigen composition.

A phosphoantigen or phosphoantigen composition is “essentially free” of another compound when the other compound(s) are present in an amount that is no more than 0.5% of the amount of the phosphoantigen composition.

A phosphoantigen or phosphoantigen composition is “free” of another compound when the other compound(s) are present in an amount that is no more than 0.1% of the amount of the phosphoantigen preparation. Alternatively, a phosphoantigen is “free” of another compound when the compound cannot be detected by HPAEC under conditions of maximum sensitivity in which a limit of detection is approximately 0.05% or less of the amount of the phosphoantigen preparation. Exemplary HPAEC methods are described herein in the section titled “Examples”.

“Purified” phosphoantigen or phosphoantigen composition refers to substantially pure phosphoantigen, essentially pure phosphoantigen, or a salt thereof, or to phosphoantigen, or a salt thereof which is substantially free, essentially free, or free of another compound.

“Partially purified” phosphoantigen or phosphoantigen composition refers to phosphoantigen, or a salt thereof that is less than 90% pure.

The purity of phosphoantigen or another compound refers to the phosphoantigen or other compound prior to its formulation in a pharmaceutical composition. The purity may be measured by any means including nuclear magnetic resonance (NMR), liquid chromatography/mass spectroscopy (LC/MS) or microbiological assays. A preferred means for measuring the purity of phosphoantigen is by analytical high pressure liquid chromatography such as HPLC or HPAEC which measures the anionic purity.

Organic or inorganic bases include but are not limited to natural alkaloids of Quinquina, including but not limited to quinine, cinchonidine, cinchonine, and quinidine; quinoline bases, including but not limited to 8-hydroxy-quinoline and 5-chloro-8-hydroxy-quinoline; and pharmaceutically acceptable bases, including but not limited to benzathin, procain, N-methyl-D-glucamine, diethylamine, ethylenediamine, piperazine, ethanolamine, thiethanolamine, betaine, as well as basic and polar amino-acids, for example lysine and arginine, and, in general terms, any basic species that will form a thermodynamically stable crystalline (salt) phase upon reaction with an acidic phosphoantigen.

The term “organic solvent” includes, but is not limited to, 1,4-dioxane (dioxane), 1,2-dichloroethane, dimethoxyethane, diethylene glycol, dimethyl ether, tetrahydrofuran, diisopropyl ether, hydrocarbons such as hexane, heptane, cyclohexane, toluene or ethylene, alcohols such as methanols, ethanols, 1-propanol, 2-propanol, 1-butanol, 2-butanol, tert-butanol or ethylene glycol, ketones such as methyl ethyl ketone or isobutyl methyl ketone, amides such as dimethylformamide, dimethylacetamide or N-methylpyrrolidone, and mixtures thereof.

As used herein, the term “pharmaceutically acceptable salt” refers to a salt prepared from pharmacologically acceptable cations, such as, but not limited to, pharmacologically acceptable metal cations, ammonium, amine cations, or quaternary ammonium cations. Especially preferred metal cations are those derived from the alkali metals, e.g., lithium, sodium, and potassium, and from the alkaline earth metals, magnesium and calcium, although cationic forms of other metals, e.g., aluminum, zinc, and iron are also within the scope of this invention. Pharmacologically acceptable amine cations are those derived from primary, secondary, and tertiary amines, particularly N-methylglucamine (meglumine), and the lower alkanolammonium and other base salts of pharmaceutically acceptable organic amines.

As used herein, the term “crystalline phase” means a composition with a distinct crystal packing.

As used herein, the term “polymorph” means crystalline phases of the same composition that differ by their crystal packing.

As used herein the term “solvate” is a crystalline complex of fixed or variable stoichiometry formed by a solute (e.g. phosphoantigen or salts) and a solvent. Several solvated phases with the same composition could exist, representing polymorphic forms of solvated phases.

As used herein, the term “therapeutically effective amount” refers to that amount of a therapy (e.g., a therapeutic agent) which is sufficient to ameliorate a disease or condition, or one or more symptoms thereof, or prevent the advancement of the disease or condition, or improve the therapeutic effect(s) of another therapy (e.g., a therapeutic agent or other physical treatment).

As used herein, the terms “treat”, “treatment” and “treating” refer to the reduction or amelioration of the progression, severity, and/or duration of a disease or condition, or one or more symptoms thereof that results from the administration of one or more therapies (e.g., one or more prophylactic and/or therapeutic agents).

As used herein, the terms “prevent”, “preventing”, and “prevention” refer to the prevention of the recurrence, onset, or development of a disease or condition, or one or more symptoms, thereof in a subject, said prevention resulting from a therapy (e.g., the administration of a prophylactic or therapeutic agent), or a combination therapy (e.g., the administration of a combination of prophylactic or therapeutic agents).

As used herein, “treating cancer” includes preventing the development of a cancer, reducing the symptoms of cancer, and/or inhibiting the growth, reducing the size and/or inducing the destruction of an established cancer. In other aspects, a medicament is administered to a subject at risk of developing a cancer for the purpose of reducing the risk of developing the cancer.

As used herein, the term “adjunctively administered” refers to the administration of one or more compounds or active ingredients in addition to a pharmaceutically acceptable phosphoantigen composition, either simultaneously or at intervals prior to, during, or following administration of the phosphoantigen composition to achieve the desired therapeutic or prophylactic effect.

In the framework of the present invention, the expression “Formulas I to III”, designate all compounds derived from Formulas I to III: I, II, IIa, II, IIIa, IIIa1, IIIa2, IIIa3, A, B, IIIb, IIIb1, IIIb2, IIIb3, C, IIIc, IIIc1, IIIc2, IIIc3, D, E, F and G.

### Description of General Method

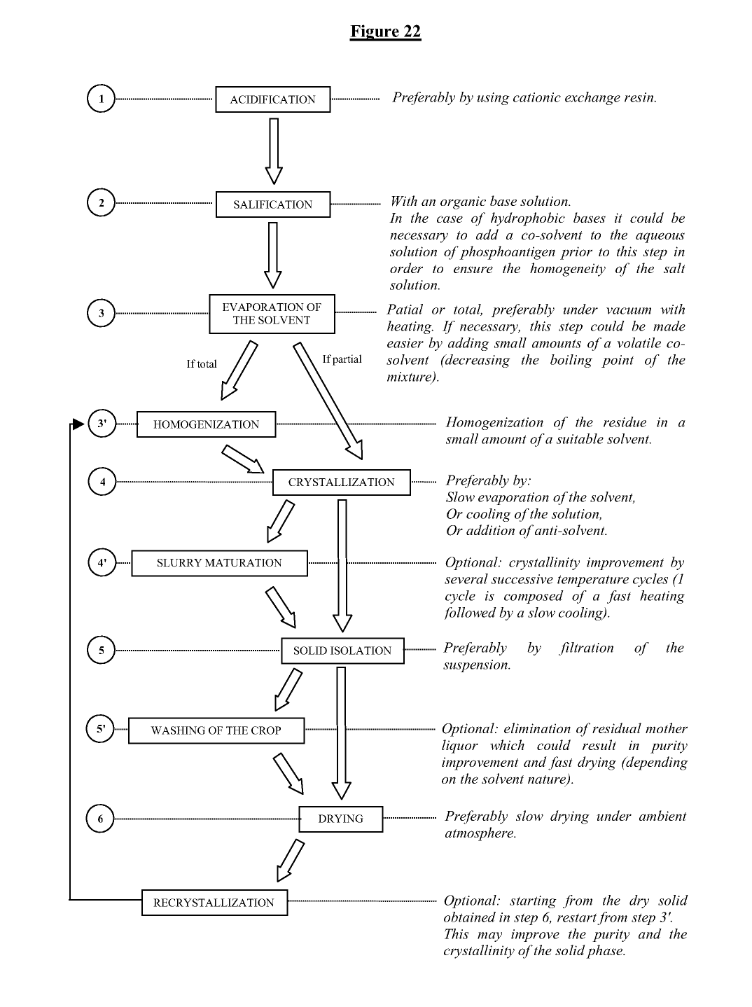

The method that can be used in a general fashion to prepare crystalline phosphoantigens is set forth in FIG. 22, and further described as follows. Furthermore, detailed protocol for such method is provided in the Examples section. It will be appreciated that modifications can be made to adapt the process for each particular phosphoantigen. For purposes of exemplifying the type of modifications, a number of different phosphoantigens are crystallized in the Examples, with the use of a number of different exemplary organic bases.

### Method for Crystallizing Phosphoantigen Compounds the Starting Material

Phosphoantigens suitable for use in the present methods are set forth in further detail below. Starting material is preferably provided in a sufficiently stable form and if possible in acid form, or as a salt of a monovalent mineral cation such as sodium, potassium, lithium or ammonium, such monovalent mineral cations being more readily exchangeable on resin. In certain cases where compounds are already available in acid form, as with some bisphosphonic acids, the acidification (step 1) is not required.

### Phosphoantigens

The term “phosphoantigen” designates a compound comprising a phosphate moiety. In some cases phosphoantigens have an immuno-modulatory activity, e.g. preferably are able to activate or stimulate an immune cell, either directly or indirectly. A preferred non-limiting example is a phosphoantigen that activates or stimulates an immune cell (for example a T cell, particularly a γδT cell, (e.g. Vγ9Vδ2 T cell), an NK cell, a dendritic cell). For this reason, phosphoantigens that have the property, for example, of activating or stimulating γδT cells are referred to as “γδT cell activator”. A γδT cell activator is preferably but not necessarily a ligand of the T receptor of γδT lymphocytes, even when it acts directly on γδT lymphocytes. Other examples include a ligand (or part of a complex with a ligand, or involved in a signalling pathway) of a Toll-Like Receptor (TLR). It will be appreciated, that a γδT cell activator may stimulate or activate a γδT cell directly or indirectly, the latter generally occurring via stimulating or activation of another immune cell which in turn leads to activation of a γδT cell. Also suitable for use in the present methods are phosphoantigen compounds (i.e., that containing a phosphate moiety) and that are structurally related to the phosphoantigens that activate or stimulate γδT cells, but that do not themselves activate or stimulate γδT cells. Examples of such compounds include nucleotides and derivatives or analogs thereof which do or do not have immuno-modulatory activity. Also suitable for use in the present methods are any compounds containing a phosphate moiety that are structurally related to the phosphoantigens that activate or stimulate γδT cells but are adapted to act as inhibitors of γδT cells, referred to as a “γδT cell inhibitor”. Examples of γδT cell inhibitors are disclosed in U.S. Pat. No. 6,624,151, the disclosure of which is incorporated herein by reference.

A phosphoantigen that is a γδT cell activator preferably increases the biological activity or causes the proliferation of γδT cells, preferably increasing the activation of γδT cells, particularly increasing cytokine secretion from γδT cells or increasing the cytolytic activity of γδT cells, with or without also stimulating the proliferation or expansion of γδT cells. Accordingly, the γδT cell activator is administered in an amount and under conditions sufficient to increase the activity γδT cells in a subject, preferably in an amount and under conditions sufficient to increase cytokine secretion by γδT cells and/or to increase the cytolytic activity of γδT cells. Cytokine secretion and cytolytic activity can be assessed using any appropriate in vitro assay.

In any exemplary assay, cytokine secretion can be determined according to the methods described in Espinosa et al. (J. Biol. Chem., 2001, Vol. 276, Issue 21, 18337-18344), describing measurement of TNF-α release in a bioassay using TNF-α-sensitive cells. Briefly, 10⁴ γδT cells/well were incubated with stimulus plus 25 units of IL2/well in 100 μl of culture medium during 24 h at 37° C. Then, 50 μl of supernatant were added to 50 μl of WEHI cells plated at 3×10⁴ cells/well in culture medium plus actinomycin D (2 μg/ml) and LiCl (40 mM) and incubated for 20 h at 37° C. Viability of the TNF-α-sensitive cells and measured with a 3-(4,5-dimethylthiazol-2-yl)-2,5-diphenyltetrazolium bromide assay. 50 μl of 3-(4,5-dimethylthiazol-2-yl)-2,5-diphenyltetrazolium bromide (Sigma; 2.5 mg/ml in phosphate-buffered saline) per well were added, and after 4 h of incubation at 37° C., 50 μl of solubilization buffer (20% SDS, 66% dimethyl formamide, pH 4.7) were added, and absorbance (570 nm) was measured. Levels of TNF-α release were then calculated from a standard curve obtained using purified human rTNF-α (PeproTech, Inc., Rocky Hill, N.J.). Interferon-γ released by activated T cells was measured by a sandwich enzyme-linked immunosorbent assay. 5×10⁴ T cells/well were incubated with stimulus plus 25 units of IL2/well in 100 μl of culture medium during 24 h at 37° C. Then, 50 μl of supernatant were harvested for enzyme-linked immunosorbent assay using mouse monoclonal antibodies (BIOSOURCE, Camarillo, Calif.).

A preferred assay for cytolytic activity is a ⁵¹Cr release assay. In exemplary assays, the cytolytic activity of γδT cells is measured against autologous normal and tumor target cell lines, or control sensitive target cell lines such as Daudi and control resistant target cell line such as Raji in 4 h ⁵¹Cr release assay. In a specific example, target cells were used in amounts of 2×10³ cells/well and labeled with 100 μCi ⁵¹Cr for 60 minutes. Effector/Target (E/T) ratio ranged from 30:1 to 3.75:1. Specific lysis (expressed as percentage) is calculated using the standard formula [(experimental-spontaneous release/total-spontaneous release)×100].

As discussed, the methods of the invention can generally be carried out with any γδT cell activator that is capable of stimulating γδT cell activity. This stimulation can be by direct effect on γδT cells as discussed below using compounds that can stimulate γδT cells in a pure γδT cell culture, or the stimulation can be by an indirect mechanism, such as treatment with pharmacological agents such as bisphosphonates which lead to IPP accumulation. Preferably, a γδT cell activator is a compound capable of regulating the activity of a γδT cell in a population of γδT cell clones in culture. The γδT cell activator is capable of regulating the activity of a γδT cell population of γδT cell clones at millimolar concentration, preferably when the γδT cell activator is present in culture at a concentration of less than 100 mM. Optionally a γδT cell activator is capable of regulating the activity of a γδT cell in a population of γδT cell clones at millimolar concentration, preferably when the γδT cell activator is present in culture at a concentration of less than 10 mM, or more preferably less than 1 mM. Regulating the activity of a γδT cell can be assessed by any suitable means, preferably by assessing cytokine secretion, most preferably TNF-α secretion as described herein. Methods for obtaining a population of pure γδT cell clones is described in Davodeau et al, (1993) and Moreau et al, (1986), the disclosures of which are incorporated herein by reference. Preferably the activator is capable of causing at least a 20%, 50% or greater increase in the number of γδT cells in culture, or more preferably at least a 2-fold increase in the number of γδT cells in culture.

In one embodiment, the activator may be a synthetic chemical compound capable of selectively activating Vγ9Vδ2 T lymphocytes. Selective activation of Vγ9Vγ2 T lymphocytes indicates that the compound has a selective action towards specific cell populations, preferably increasing activation of Vγ9Vδ2 T cells at a greater rate or to a greater degree than other T cell types such as Vδ1 T cells, or not substantially not activation other T cell types. Such selectivity can be assessed in vitro T cell activation assays. Such selectivity, as disclosed in the present application, suggests that preferred compounds can cause a selective or targeted activation of the proliferation or biological activity of Vγ9Vδ2 T lymphocytes.

### Preferred Phosphoantigens

In a preferred embodiment, the phosphoantigen is a compound of Formula I, especially a γδT cell activator according to Formulas I to III, especially γδT cell activator selected from the group consisting of BrHPP, CBrHPP, IPP, epoxPP, HDMAPP, C-HDMAPP, N-HDMAPP and H-angelylPP (compounds A to G). In the framework of the present invention, the expression “Formulas I to III”, designate all compounds derived from Formulas I to III: I, II, IIa, III, IIIa, IIIa1, IIIa2, IIIa3, A, B, IIIb, IIIb1, IIIb2, IIIb3, C, IIIc, IIIc1, IIIc2, IIIc3, D, E, F and G. Most preferably, the compounds are selected from the list consisting of BrHPP, IPP, HDMAPP, C-HDMAPP, N-HDMAPP and H-angelylPP. However, it will appreciated that a number of phosphoantigen compounds that are less potent γδT cell activators are available and may be used in accordance with the invention. For example, in one variant, a bisphosphonate compounds such as pamidronate (Novartis, Nuernberg, Germany) or zoledronate may be used. Other γδT cell activators for use in the present invention are phosphoantigens disclosed in WO 95/20673, isopentenyl pyrophosphate (IPP) (U.S. Pat. No. 5,639,653), the disclosures of the two preceding documents being incorporated herein by reference, as well as alkylamines (such as ethylamine, iso-propyulamine, n-propylamine, n-butylamine and iso-butylamine, for instance). Isobutyl amine and 3-aminopropyl phosphonic acid are obtained from Aldrich (Chicago, Ill.).

In one aspect, a phosphoantigen according to the present invention comprises a compound of Formula (I):

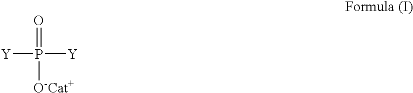

wherein Cat⁺ represents one (or several, identical or different) organic or mineral cation(s) (including proton);
Y represents O⁻Cat⁺, a C₁-C₆, or more preferably C₁-C₃, alkyl group, a group —B—R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
B represents O, NH, CHF, CF₂ or CH₂ or R₁—C—R₂ wherein R₁ and R₂ are defined as R but may vary independently of each other, respectively; and,
R is a linear, branched, or cyclic, aromatic or not, saturated or unsaturated, C₁-C₂₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof. Preferably, R₁ and R₂ are defined as any of the R₁ and R₂ shown in Table 1.

In another aspect, a phosphoantigen according to the present invention comprises a compound of Formula (II):

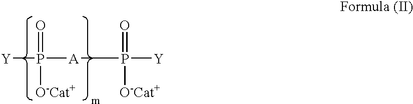

wherein Cat⁺ represents one (or several, identical or different) organic or mineral cation(s) (including proton);
m is an integer from 1 to 3;
A is O, NH, or any group capable to be hydrolyzed;
Y and B have the aforementioned meaning.

The term “independently” is used in the present specification to indicate that the variable which is independently applied varies independently from application to application. Thus, in a compound such as R—C—R, wherein “R varies independently as carbon or nitrogen”, both R can be carbon, both R can be nitrogen, or one R can be carbon and the other R nitrogen.

In further embodiments it will be appreciated that the present invention and particularly method for making crystalline phases is suitable for use with structurally related compounds to the ones mentioned specifically herein. In preferred embodiments, the invention also encompasses nucleotides and nucleotide analogs or derivatives or nucleotide-like compounds as well as a bisphosphonate compounds. In a preferred aspect, a compound of Formula II is a bisphosphonate compound, preferably a compound of Formula IIa. A bisphosphonate compound preferably comprises a structure of the Formula (IIa):

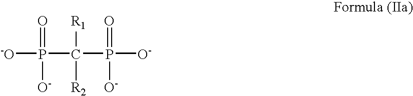

Preferably, R₁ and R₂ are defined as any of the R₁ and R₂ shown in Table 1.

TABLE 1

| Agent | R₁ side chain | R₂ side chain |
|---|---|---|
| Etidronate | —OH | —CH₃ |
| Clodronate | —Cl | —Cl |
| Tiludronate | —H | 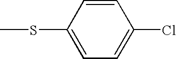 |
| Pamidronate | —OH | —CH₂—CH₂—NH₂ |
| Neridronate | —OH | —(CH₂)₅—NH₂ |
| Olpadronate | —OH | —(CH₂)₂—N(CH₃)₂ |
| Alendronate | —OH | —(CH₂)₃—NH₂ |
| Ibandronate | —OH | 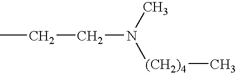 |
| Risedronate | —OH | 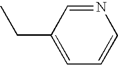 |
| Zoledronate | —OH | 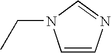 |

Preferably, a compound of the bisphosphonate type is selected from the group consisting of the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-ethidene-bisphosphonic acid, e.g. etidronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-pentylamino)-1-hydroxypropane-1,1-diphosphonic acid, e.g. methyl-pentyl-APD (=BM 21.0955); 1-hydroxy-2-(imidazol-1yl)ethane-11,-diphosphonic acid; 1-hydroxy-2-(3-pyridinyl)ethane-1,1-diphosphonic acid (risedronic acid), e.g. risedronate, including N-methylpyridinium salts thereof, for example N-methylpyridinium iodides such as NE-10244 or NE-10446, 1-(4-chlorophenylthio)methane-1,1-diphosphonic acid (tiludronic acid), e.g. tiludronate; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); 1-hydroxy-2-(imidazo[1,2-a]pyridin-3-yl)ethane-1,1-diphosphonic acid, e.g. YM 529; and 1,1-dichloromethane-1,1-diphosphonic acid (clodronic acid), e.g. clodronate. Preferably the bisphosphonate are compounds which lead to activation of γδT cells. Examples of commercialised bisphosphonates are shown in Table 1 above, including the identity of R₁ and R₂ for each molecule.

Nucleotides and nucleotide analogs or derivatives or nucleotide-like compounds are also expected to be compatible with the methods of the invention. The term “nucleotide” is used to refer to a molecule that including a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). Nucleotides also include substituted purines and pyrimidines such as C₅-propyne; pyrimidine and 7-deaza-7-substituted purine modified bases (Wagner R W et al. (1996) Nat; Biotechnol 14:840-4). Purines and pyrimidines include but are not limited to adenine, cytosine, guanine, thymine, and uracil, and other naturally and non-naturally occurring nucleobases, substituted and unsubstituted aromatic moieties. The term “nucleotide” shall also include nucleotides having one, two or three phosphate groups (e.g. a nucleoside having one, two or three phosphate groups) and any other organic base containing polymer, e.g. including deoxyribonucleoside triphosphates such as dATP, dCTP, dITP, dUTP, dGTP, dTTP, deoxyribonucleoside diphosphates such as dADP, dCDP, dIDP, dUDP, dGDP, dTDP, deoxyribonucleoside monophosphates such as dAMP, dCMP, dIMP, dUMP, dGMP, dTMP and for example [αS]dATP, 7-deaza-dGTP and 7-deaza-dATP. The term “nucleotide” as used herein also refers to ribonucleoside triphosphates (e.g. rNTPs), diphosphates and monophospages and their derivatives. Illustrated examples of ribonucleoside triphosphates include, but are not limited to: ATP, CTP, GTP, ITP and UTP. A modified base is any base which is chemically distinct from the naturally occurring bases typically found in DNA and RNA such as T, C, G, A and U, but which share basic chemical structures with these naturally occurring bases. The modified nucleoside base may be, for example, selected from hypoxanthine, uracil, dihydrouracil, pseudouracil, 2-thiouracil, 4-thiouracil, 5-aminouracil, 5-(C₂-C₆)-alkyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₂-C₆)-alkylcytosine, 5-(C₂-C₆)-alkenylcytosine, 5-(C₂-C₆)-alkynylcytosine, 5-chlorocytosine, 5-fluorocytosine, 5-bromocytosine, (N)-2-dimethylguanine, 2,4-diamino-purine, 8-azapurine, a substituted 7-deazapurine, preferably; 7-deaza-7-substituted and/or 7-deaza-8-substituted purine, 5-hydroxymethylcytosine, (N)-4-alkylcytosine, e.g., (N)-4-ethylcytosine, 5-hydroxydeoxycytidine, (S)-hydroxymethyldeoxycytidine, (N)-4-alkyldeoxycytidine, e.g., (N)-4-ethyldeoxycytidine, 6-thiodeoxyguanosine, and deoxyribonucleosides of nitropyrrole. C₅-propynylpyrimidine, and diaminopurine e.g., 2,6-diaminopurine, inosine, 5-methylcytosine, 2-aminopurine, 2-amino-6-chloropurine, hypoxanthine or other modifications of a natural nucleoside bases.

Thus, nucleosides and nucleoside analogs may be used in accordance with the invention, generally as long as the nucleoside or nucleoside analog comprises at least one phosphate. For example, ANA317 (LB80317) and its ester prodrug version, LB80380/ANA380, are phosphonate nucleoside analogue of guanosine monophosphate. Another example is MCC-478 (LY582563), a nucleoside analogue derivative of phosphonomethoxyethyl purine. MCC0478 has the structure 2-amino-9-[2-(phosphonomethoxy)ethyl]-6-(4-methoxyphenylthio)purine bis(2,2,2-trifluoroethyl) ester and has been useful in treatment of wild-type and lamivudine-resistant HBV. Yet another example is Adefovir Dipivoxil. Adefovir dipivoxil is the acyclic analogue of dAMP. The chemical structures of MCC-478 (A) and adefovir (B) are disclosed in Kioko Ono-Nita et al., Antimicrobial Agents and Chemotherapy, August 2002, p. 2602-2605, Vol. 46, No. 8, the disclosure of the chemical structure and properties of the compound being incorporated herein by reference. Yet another example is tenofovir disoproxil fumarate. Tenofovir is an acyclic nucleotide inhibitor of HBV polymerase and HIV reverse transcriptase, with close chemical similarity to adefovir dipivoxil. Examples of analogs which may be modified so as to contains a phosphate can include for example Lamivudine, Entecavir, Emtricitabine, Elvucitabine and 2′,3′-dideoxy-3′-fluoroguanosine (FLG). Entecavir is a cyclopentyl guanosine analog that selectively inhibits HBV replication; Emtricitabine (FTC) is a fluorinated cytosine analogue (Billich, A, Curr Opin Investig Drugs. 2001 May; 2(5):617-21); Elvucitabine is an L-configuration cytidine analog; 2′,3′-dideoxy-3′-fluoroguanosine (FLG) is a deoxyguanosine analogue that is inhibitory to both HIV-1 and HBV. Other examples include the L-nucleoside analogues, L-deoxythymidine (LdT; telbivudine) and valyl-L-deoxycytidine (val-LdC; valtorcitabine), are very promising compounds that are potent and selective inhibitors of HBV replication. Further examples include imidazoquinoline amines, imiquimod (chemical name 1-(2-amino-2-methylpropyl)-2-(ethoxymethyl)-1H-imidazo[4,5-c]quinolin-4-amine or 4-amino-1-isobutyl-1H-imidazo[4,5-c]quinoline, bropirimine, imidazoquinoline and isatoribine, particularly those that stimulate at least NK cells and/or dendritic cells directly via toll-like receptor (TLR) mediated signalling. Examples of nucleic acid based compound are believed to act through other TLR receptors, such as CpG oligonucleotides acting via TLR9. Further examples include guanosine analogs such as loxoribine (7-allyl-8-oxoguanosine) Pope et al, Cellular Immunology 1995, 162:2(333-339) and Pope et al. Journal of Immunology 1993, 151:6(3007-3017.

Further examples of preferred phosphoantigens for use according to the present invention comprise the compounds of Formula (III):

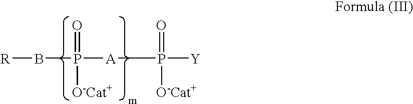

wherein:
Cat⁺ represents one (or several, identical or different) organic or mineral cation(s) (including proton);
m is an integer from 1 to 3;
A is O, NH, or any group capable to be hydrolyzed;
Y represents O⁻Cat⁺, a C₁-C₃ alkyl group, a group —B—R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
B is O, NH, CHF, CF₂ or CH₂; and,
R is a linear, branched, or cyclic, aromatic or not, saturated or unsaturated, C₁-C₂₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof. Most preferably, said phosphoantigen compound are γδT cell activators.

In a particular embodiment of any of the formulas disclosed for use in accordance with the invention, the substituents as defined above are substituted by at least one of the substituents as specified above.

Preferably, the substituents are selected from the group consisting of: an (C₁-C₆)alkyl, an (C₂-C₆)alkylenyl, an (C₂-C₆)alkynyl, an (C₂-C₆)epoxyalkyl, an aryl, an heterocycle, an (C₁-C₆)alkoxy, an (C₂-C₆)acyl, an (C₁-C₆)alcohol, a carboxylic group (—COOH), an (C₂-C₆)ester, an (C₁-C₆)amine, an amino group (—NH₂), an amide (—CONH₂), an (C₁-C₆)imine, a nitrile, an hydroxyl (—OH), an aldehyde group (—CHO), an halogen, an (C₁-C₆)halogenoalkyl, a thiol (—SH), a (C₁-C₆)thioalkyl, a (C₁-C₆)sulfone, a (C₁-C₆)sulfoxide, and a combination thereof.

More preferably, the substituents are selected from the group consisting of: an (C₁-C₆)alkyl, an (C₂-C₆)epoxyalkyl, an (C₂-C₆)alkylenyl, an (C₁-C₆)alkoxy, an (C₂-C₆)acyl, an (C₁-C₆)alcohol, an (C₂-C₆)ester, an (C₁-C₆)amine, an (C₁-C₆)imine, an hydroxyl, a aldehyde group, an halogen, an (C₁-C₆)halogenoalkyl and a combination thereof.

Still more preferably, the substituents are selected from the group consisting of: an (C₃-C₆)epoxyalkyl, an (C₁-C₃)alkoxy, an (C₂-C₃)acyl, an (C₁-C₃)alcohol, an (C₂-C₃)ester, an (C₁-C₃)amine, an (C₁-C₃)imine, an hydroxyl, an halogen, an (C₁-C₃)halogenoalkyl, and a combination thereof. Preferably, R is a (C₃-C₂₅)hydrocarbon group, more preferably a (C₅-C₁₀)hydrocarbon group.

In the context of the present invention, the term “alkyl” more specifically means a group such as methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, docosyl and the other isomeric forms thereof. (C₁-C₆)alkyl more specifically means methyl, ethyl, propyl, isopropyl, butyl, isobutyl, tert-butyl, pentyl, hexyl and the other isomeric forms thereof. (C₁-C₃)alkyl more specifically means methyl, ethyl, propyl, or isopropyl.

The term “alkenyl” refers to an alkyl group defined hereinabove having at least one unsaturated ethylene bond and the term “alkynyl” refers to an alkyl group defined hereinabove having at least one unsaturated acetylene bond. (C₂-C₆)alkylene includes a ethenyl, a propenyl (1-propenyl or 2-propenyl), a 1- or 2-methylpropenyl, a butenyl (1-butenyl, 2-butenyl, or 3-butenyl), a methylbutenyl, a 2-ethylpropenyl, a pentenyl (1-pentenyl, 2-pentenyl, 3-pentenyl, 4-pentenyl), an hexenyl (1-hexenyl, 2-hexenyl, 3-hexenyl, 4-hexenyl, 5-hexenyl), and the other isomeric forms thereof. (C₂-C₆)alkynyl includes ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl and the other isomeric forms thereof.

The term “epoxyalkyl” refers to an alkyl group defined hereinabove having an epoxide group. More particularly, (C₂-C₆)epoxyalkyl includes epoxyethyl, epoxypropyl, epoxybutyl, epoxypentyl, epoxyhexyl and the other isomeric forms thereof. (C₂-C₃)epoxyalkyl includes epoxyethyl and epoxypropyl.

The “aryl” groups are mono-, bi- or tri-cyclic aromatic hydrocarbons having from 6 to 18 carbon atoms. Examples include a phenyl, α-naphthyl, β-naphthyl or anthracenyl group, in particular.

“Heterocycle” groups are groups containing 5 to 8 rings comprising one or more heteroatoms, preferably 1 to 5 endocyclic heteroatoms. They may be mono-, bi- or tri-cyclic. They may be aromatic or not. Preferably, and more specifically for R₅, they are aromatic heterocycles. Examples of aromatic heterocycles include pyridine, pyridazine, pyrimidine, pyrazine, furan, thiophene, pyrrole, oxazole, thiazole, isothiazole, imidazole, pyrazole, oxadiazole, triazole, thiadiazole and triazine groups. Examples of bicycles include in particular quinoline, isoquinoline and quinazoline groups (for two 6-membered rings) and indole, benzimidazole, benzoxazole, benzothiazole and indazole (for a 6-membered ring and a 5-membered ring). Nonaromatic heterocycles comprise in particular piperazine, piperidine, etc.

“Alkoxy” groups correspond to the alkyl groups defined hereinabove bonded to the molecule by an —O— (ether) bond. (C₁-C₆)alkoxy includes methoxy, ethoxy, propyloxy, butyloxy, pentyloxy, hexyloxy and the other isomeric forms thereof. (C₁-C₃)alkoxy includes methoxy, ethoxy, propyloxy, and isopropyloxy.

“Alkyl” groups correspond to the alkyl groups defined hereinabove bonded to the molecule by an —CO— (carbonyl) group. (C₂-C₆)acyl includes acetyl, propylacyl, butylacyl, pentylacyl, hexylacyl and the other isomeric forms thereof. (C₂-C₃)acyl includes acetyl, propylacyl and isopropylacyl.

“Alcohol” groups correspond to the alkyl groups defined hereinabove containing at least one hydroxyl group. Alcohol can be primary, secondary or tertiary. (C₁-C₆)alcohol includes methanol, ethanol, propanol, butanol, pentanol, hexanol and the other isomeric forms thereof. (C₁-C₃)alcohol includes methanol, ethanol, propanol and isopropanol.

“Ester” groups correspond to the alkyl groups defined hereinabove bonded to the molecule by an —COO— (ester) bond. (C₂-C₆)ester includes methylester, ethylester, propylester, butylester, pentylester and the other isomeric forms thereof. (C₂-C₃)ester includes methylester and ethylester.

“Amine” groups correspond to the alkyl groups defined hereinabove bonded to the molecule by an —N— (amine) bond. (C₁-C₆)amine includes methylamine, ethylamine, propylamine, butylamine, pentylamine, hexylamine and the other isomeric forms thereof. (C₁-C₃)amine includes methylamine, ethylamine, and propylamine.

“Imine” groups correspond to the alkyl groups defined hereinabove having a (—C═N—) bond. (C₁-C₆)imine includes methylimine, ethylimine, propylimine, butylimine, pentylimine, hexylimine and the other isomeric forms thereof. (C₁-C₃)imine includes methylimine, ethylimine, and propylimine.

The halogen can be Cl, Br, I, or F, more preferably Br or F.

“Halogenoalkyl” groups correspond to the alkyl groups defined hereinabove having at least one halogen. The groups can be monohalogenated or polyhalogenated containing the same or different halogen atoms. For example, the group can be a trifluoroalkyl (CF₃—R). (C₁-C₆)halogenoalkyl includes halogenomethyl, halogenoethyl, halogenopropyl, halogenobutyl, halogenopentyl, halogenohexyl and the other isomeric forms thereof. (C₁-C₃)halogenoalkyl includes halogenomethyl, halogenoethyl, and halogenopropyl.

“Thioalkyl” groups correspond to the alkyl groups defined hereinabove bonded to the molecule by an —S— (thioether) bond. (C₁-C₆)thioalkyl includes thiomethyl, thioethyl, thiopropyl, thiobutyl, thiopentyl, thiohexyl and the other isomeric forms thereof. (C₁-C₃)thioalkyl includes thiomethyl, thioethyl, and thiopropyl.

“Sulfone” groups correspond to the alkyl groups defined hereinabove bonded to the molecule by an —SOO— (sulfone) bond. (C₁-C₆)sulfone includes methylsulfone, ethylsulfone, propylsulfone, butylsulfone, pentylsulfone, hexylsulfone and the other isomeric forms thereof. (C₁-C₃)sulfone includes methylsulfone, ethylsulfone and propylsulfone.

“Sulfoxyde” groups correspond to the alkyl groups defined hereinabove bonded to the molecule by an —SO— (sulfoxide) group. (C₁-C₆)sulfoxide includes methylsulfoxide, ethylsulfoxide, propylsulfoxide, butylsulfoxide, pentylsulfoxide, hexylsulfoxide and the other isomeric forms thereof. (C₁-C₃)sulfoxide includes methylsulfoxide, ethylsulfoxide, propylsulfoxide and isopropylsulfoxide.

“Heteroatom” denotes N, S, or O.

“Nucleoside” refers to a compound composed of any pentose or modified pentose moiety attached to a specific position of a heterocycle or to the natural positions of a purine (9-position) or pyrimidine (1-position) or to the equivalent position in an analog. The term nucleoside includes but is not limited to adenosine, thymine, uridine, cytidine and guanosine.

In a particular embodiment, the hydrocarbon group is a cycloalkylenyl such as a cyclopentadiene or a phenyl, or an heterocycle such as a furan, a pyrrole, a thiophene, a thiazole, an imidazole, a triazole, a pyridine, a pyrimidine, a pyrane, or a pyrazine. Preferably, the cycloalkylenyl or the heterocycle is selected from the group consisting of a cyclopentadiene, a pyrrole or an imidazole. In a preferred embodiment, the cycloalkylenyl or the heterocycle is substituted by an alcohol. Preferably, said alcohol is a (C₁-C₃)alcohol.

In another embodiment, the hydrocarbon group is an alkylenyl with one or several double bonds. Preferably, the alkylenyl group has one double bond. Preferably, the alkylenyl group is a (C₃-C₁₀)alkylenyl group, more preferably a (C₄-C₇)alkylenyl group. Preferably, said alkylenyl group is substituted by at least one functional group. More preferably, the functional group is selected from the group consisting of an hydroxy, an (C₁-C₃)alkoxy, an aldehyde, an (C₂-C₃)acyl, or an (C₂-C₃)ester. In a more preferred embodiment, the hydrocarbon group is butenyl substituted by a group —CH₂OH. Optionally, said alkenyl group can be the isoform trans (E) or cis (Z), more preferably a trans isoform (E). In a most preferred embodiment, the alkylenyl group is the (E)-4-hydroxy-3-methyl-2-butenyl. In an other preferred embodiment, the alkylenyl group is an isopentenyl, a dimethylallyl or an hydroxydimethylallyl.

In an additional embodiment, the hydrocarbon group is an alkyl group substituted by an acyl. More preferably, the hydrocarbon group is an (C₄-C₇)alkyl group substituted by an (C₁-C₃)acyl.

In a further preferred embodiment, the phosphoantigen is of formula (IIa):

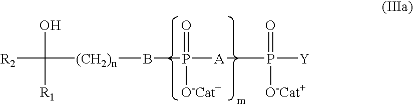

in which:
R₂ is an halogenated (C₁-C₃)alkyl, a (C₁-C₃)alkoxy-(C₁-C₃)alkyl, an halogenated (C₂-C₃)acyl or a (C₁-C₃)alkoxy-(C₂-C₃)acyl,
R₁ is (C₁-C₃)alkyl group,
m is an integer from 1 to 3,
n is an integer from 2 to 20,
A represents O, NH, or any group capable to be hydrolyzed
B represents O, NH, CHF, CF₂ or CH₂,
Y represents O⁻Cat⁺, a C₁-C₃ alkyl group, a group —B—R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
R is a linear, branched, or cyclic, aromatic or not, saturated or unsaturated, C₁-C₅₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof. Most preferably, said phosphoantigen compound are γδT cell activators, and
Cat⁺ represents one (or several, identical or different) organic or mineral cation(s) (including the proton).
   Preferably, R₂ is an halogenated methyl (—CH₂—X, X being an halogen), an halogenated (C₂-C₃)acetyl, or (C₁-C₃)alkoxy-acetyl. The halogenated methyl or acetyl can be mono-, di-, or tri-halogenated. More preferably, R₂ is a CH₂—X group, X represents a halogen atom. Preferably, R₁ is a methyl or ethyl group. More preferably, R₁ is a methyl. Preferably, A is O and B is O or CH₂. Preferably, n is an integer from 2 to 10, or from 2 to 5. In a more preferred embodiment, n is 2. Preferably, m is 1 or 2. More preferably, m is 1. Preferably, Y is O⁻Cat⁺, or a nucleoside. More preferably, Y is O⁻Cat⁺.
   In a most preferred embodiment, n is 2, R₁ is a methyl and R₂ is a halogenated methyl, more preferably a monohalogenated methyl, still more preferably a bromide methyl. In a particularly preferred embodiment, n is 2, R₁ is a methyl, R₂ is a methyl bromide. In a most preferred embodiment, R is 3-(bromomethyl)-3-butanol-1-yl.

In another embodiment, R₂ is a CH₂—X group and A and B represent O.

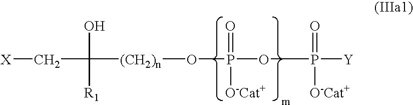

In another embodiment R₂ is a CH₂—X group and A represents O and B represents CH₂.

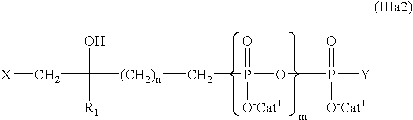

wherein R₁, X, n, m, Y and Cat⁺ have the aforementioned meaning.
In one preferred embodiment, a phosphoantigen comprises a compound of Formula (IIIa3):

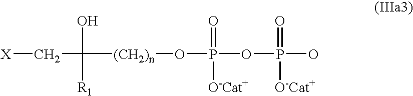

in which X is an halogen (preferably selected from I, Br and Cl), R₁ is a methyl or ethyl group, Cat⁺ represents one (or several, identical or different) organic or mineral cation(s) (including the proton), and n is an integer from 2 to 20. Preferably, R₁ is a methyl. Preferably, n is 2. Preferably, X is a bromide.

In a most preferred embodiment, a phosphoantigen comprises a compound of Formula (A):

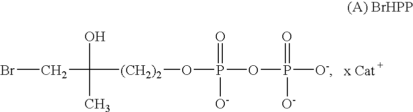

### Preferably x Cat⁺ is 1 or 2 Na⁺.

In another most preferred embodiment, a phosphoantigen comprises a compound of Formula (B):

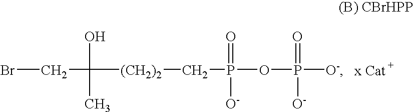

### Preferably x Cat⁺ is 1 or 2 Na⁺.

In a further preferred embodiment, the phosphoantigen is of formula (IIIb):

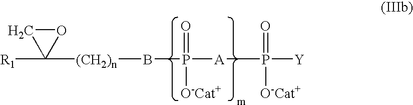

wherein:
n is an integer from 2 to 20,
m is an integer from 1 to 3,
R₁ is a methyl or ethyl group,
A represents O, NH, or any group capable to be hydrolyzed,
B represents O, NH, CHF, CF₂ or CH₂,
Y is O⁻Cat⁺, a nucleoside, or a radical —B—R, wherein R has the aforementioned meaning, and
Cat⁺ represents one (or several, identical or different) organic or mineral cation(s) (including the proton).
   Preferably, n is an integer from 2 to 10, or from 2 to 5. In a more preferred embodiment, n is 2. Preferably, R₁ is a methyl. Preferably, Y is O⁻Cat⁺, or a nucleoside. More preferably, Y is O⁻Cat⁺. Preferably, B is O, NH or CH₂. More preferably, B is O. Preferably, A is O. Preferably, m is 1 or 2. More preferably, m is 1.

For example, a phosphoantigen may comprise a compound of Formula (IIIb1) or (IIIb2):

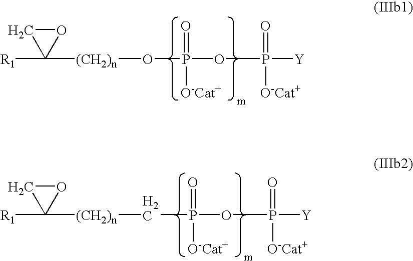

wherein R₁, n, m, Y and Cat⁺ have the above mentioned meaning.

In another preferred embodiment, a phosphoantigen comprises a compound of Formula (IIIb3):

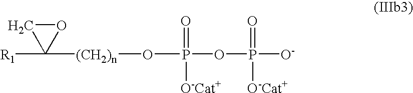

in which R₁ is a methyl or ethyl group, Cat⁺ represents one (or several, identical or different) organic or mineral cation(s) (including the proton), and n is an integer from 2 to 20. Preferably, R₁ is a methyl. Preferably, n is 2.

In another preferred embodiment, a phosphoantigen comprises a compound of Formula (C):

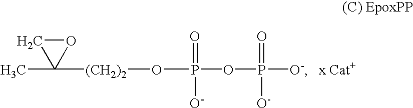

### Preferably x Cat⁺ is 1 or 2 Na⁺.

In a further preferred embodiment, the phosphoantigen is of formula (IIIc):

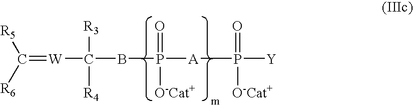

wherein:
R₃, R₅ and R₇ represent, independently from each other, a hydrogen atom or a (C₁-C₃)alkyl group,
R₆ is an (C₂-C₃)acyl, an aldehyde, an (C₁-C₃)alcohol, or an (C₂-C₃)ester,
W is —CH—, —N— or —C—R₇,
B is O, NH, CHF, CF₂ or CH₂,
A represents O, NH, or any group capable to be hydrolyzed,
m is an integer from 1 to 3,
Y is O⁻Cat⁺, a nucleoside, or a radical —B—R, wherein R has the aforementioned meaning.
Cat⁺ represents one (or several, identical or different) organic or mineral cation(s) (including the proton),

More preferably, R₃ and R₅ are a methyl and R₄ is hydrogen. More preferably, R₆ is —CH₂—OH, —CHO, —CO—CH₃ or —CO—OCH₃. Optionally, the double-bond between W and C is in conformation trans (E) or cis (Z). More preferably, the double-bond between W and C is in conformation trans (E).

The group Y can permit the design of a prodrug. Therefore, Y is enzymolabile group which can be cleaved in particular regions of the subject. The group Y can also be targeting group. In a preferred embodiment, Y is O⁻Cat⁺, a group —B—R, or a radical selected from the group consisting of a nucleoside, a monosaccharide, an epoxyde and a halohydrin. Preferably, Y is an enzymolabile group. Preferably, Y is O⁻Cat⁺, a group —B—R, or a nucleoside. In a first preferred embodiment, Y is O⁻Cat⁺. In a second preferred embodiment, Y is a nucleoside.

In a preferred embodiment, Cat⁺ is H⁺, Na⁺, NH₄⁺, K⁺, Li⁺, (CH₃CH₂)₃NH⁺.

In a preferred embodiment, A is O or NH. More preferably, A is O.

In a preferred embodiment, B is O, NH or CH₂.

In a preferred embodiment, m is 1 or 2. More preferably, m is 1.

In another example, a phosphoantigen comprises a compound of Formula (IIIc1) or (IIIc2):

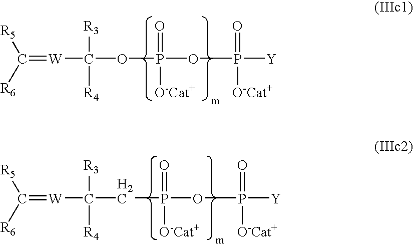

wherein R₃, R₅, R₆, R₇, W, m, Y and Cat⁺ have the above mentioned meaning.

In a preferred embodiment, W is —CH—. Preferably, R₃ and R₄ are hydrogen. Preferably, R₅ is a methyl. Preferably, R₆ is —CH₂—OH.

In another preferred embodiment, a phosphoantigen comprises a compound of Formula (D):

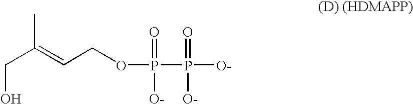

In yet another preferred embodiment, a phosphoantigen comprises a compound of Formula (E):

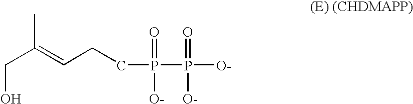

In yet another preferred embodiment, a phosphoantigen comprises a compound of Formula (F):

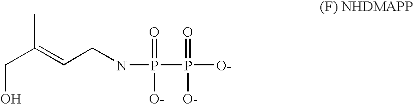

In another example, phosphoantigen comprises a compound of Formula (IIIc3):

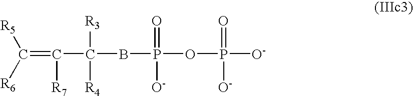

wherein R₃, R⁴, R₅, R₆, R₇, and B have the above mentioned meaning.

Preferably, R₃ and R₄ are hydrogen. Preferably, R₆ and R₇ are a methyl. Preferably, R₅ is —CH₂—OH. Preferably, B is CH₂, NH or O.

In a preferred embodiment, a phosphoantigen comprises a compound of Formula:

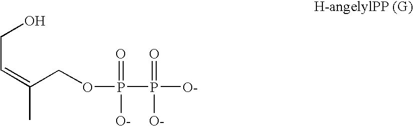

Specific examples of compounds also include: (E)1-pyrophosphonobuta-1,3-diene; (E)1-pyrophosphonopenta-1,3-diene; (E)1-pyrophosphono-4-methylpenta-1,3-diene; (E,E)1-pyrophosphono-4,8-dimethylnona-1,3,7-triene; (E,E,E)1-pyrophosphono-4,8,12-trimethyltrideca-1,3,7,11-tetraene; (E,E)1-triphosphono-4,8-dimethylnona-1,3,7-triene; 4-triphosphono-2-methylbutene; α,β-di-[3-methylpent-3-enyl]-pyrophosphonate; 1-pyrophosphono-3-methylbut-2-ene; α,γ-di-[3-methylbut-2-enyl]-triphosphonate; α,β-di-[3-methylbut-2-enyl]-pyrophosphonate; allyl-pyrophosphonate; allyl-triphosphonate; α,γ-di-allyl-pyrophosphonate; α,β-di-allyl-triphosphonate; (E,E)4-[(5′-pyrophosphono-6′-methyl-penta-2′,4′-dienyloxymethyl)-phenyl]-phenyl-methanone; (E,E)4-[(5′-triphosphono-6′-methyl-penta-2′,4′-dienyloxymethyl)-phenyl]-phenyl-methanone; (E,E,E)[4-(9′-pyrophosphono-2′,6′-dimethyl-nona-2′,6′,8′-trienyloxymethyl)-phenyl]-phenyl-methanone; (E,E,E)[4-(9′-pyrophosphono-2′,6′,8′-trimethyl-nona-2′,6′,8′-trienyloxymethyl)-phenyl]-phenyl-methanone; 5-pyrophosphono-2-methypentene; 5-triphosphono-2-methypentene; α,γ-di-[4-methylpent-4-enyl]-triphosphonate; 5-pyrophosphono-2-methypent-2-ene; 5-triphosphono-2-methypent-2-ene; 9-pyrophosphono-2,6-dimethynona-2,6-diene; 9-triphosphono-2,6-dimethynona-2,6-diene; α,γ-di-[4,8-dimethylnona-2,6-dienyl]-triphosphonate; 4-pyrophosphono-2-methylbutene; 4-methyl-2-oxa-pent-4-enyloxymethylpyrophosphate; 4-methyl-2-oxa-pent-4-enyloxymethyltriphosphate; α,β-di-[4-methyl-2-oxa-pent-4-enyloxymethyl]-pyrophosphate; and α,γ-di-[4-methyl-2-oxa-pent-4-enyloxymethyl]-triphosphate.

In other particular embodiments, the phosphoantigen can be selected from the group consisting of: 3-(halomethyl)-3-butanol-1-yl-diphosphate; 3-(halomethyl)-3-pentanol-1-yl-diphosphate; 4-(halomethyl)-4-pentanol-1-yl-diphosphate; 4-(halomethyl)-4-hexanol-1-yl-diphosphate; 5-(halomethyl)-5-hexanol-1-yl-diphosphate; 5-(halomethyl)-5-heptanol-1-yl-diphosphate; 6-(halomethyl)-6-heptanol-1-yl-diphosphate; 6-(halomethyl)-6-octanol-1-yl-diphosphate; 7-(halomethyl)-7-octanol-1-yl-diphosphate; 7-(halomethyl)-7-nonanol-1-yl-diphosphate; 8-(halomethyl)-8-nonanol-1-yl-diphosphate; 8-(halomethyl)-8-decanol-1-yl-diphosphate; 9-(halomethyl)-9-decanol-1-yl-diphosphate; 9-(halomethyl)-9-undecanol-1-yl-diphosphate; 10-(halomethyl)-10-undecanol-1-yl-diphosphate; 10-(halomethyl)-10-dodecanol-1-yl-diphosphate; 11-(halomethyl)-11-dodecanol-1-yl-diphosphate; 11-(halomethyl)-11-tridecanol-1-yl-diphosphate; 12-(halomethyl)-12-tridecanol-1-yl-diphosphate; 12-(halomethyl)-12-tetradecanol-1-yl-diphosphate; 13-(halomethyl)-13-tetradecanol-1-yl-diphosphate; 13-(halomethyl)-13-pentadecanol-1-yl-diphosphate; 14-(halomethyl)-14-pentadecanol-1-yl-diphosphate; 14-(halomethyl)-14-hexadecanol-1-yl-diphosphate; 15-(halomethyl)-15-hexadecanol-1-yl-diphosphate; 15-(halomethyl)-15-heptadecanol-1-yl-diphosphate; 16-(halomethyl)-16-heptadecanol-1-yl-diphosphate; 16-(halomethyl)-16-octadecanol-1-yl-diphosphate; 17-(halomethyl)-17-octadecanol-1-yl-diphosphate; 17-(halomethyl)-17-nonadecanol-1-yl-diphosphate; 18-(halomethyl)-18-nonadecanol-1-yl-diphosphate; 18-(halomethyl)-18-eicosanol-1-yl-diphosphate; 19-(halomethyl)-19-eicosanol-1-yl-diphosphate; 19-(halomethyl)-19-heneicosanol-1-yl-diphosphate; 20-(halomethyl)-20-heneicosanol-1-yl-diphosphate; 20-(halomethyl)-20-docosanol-1-yl-diphosphate; 21-(halomethyl)-21-docosanol-1-yl-diphosphate; and 21-(halomethyl)-21-tricosanol-1-yl-diphosphate.

More particularly, the phosphoantigen can be selected from the group consisting of: 3-(bromomethyl)-3-butanol-1-yl-diphosphate (BrHPP); 5-bromo-4-hydroxy-4-methylpentyl pyrophosphonate (CBrHPP); 3-(iodomethyl)-3-butanol-1-yl-diphosphate (IHPP); 3-(chloromethyl)-3-butanol-1-yl-diphosphate (ClHPP); 3-(bromomethyl)-3-butanol-1-yl-triphosphate (BrHPPP); 3-(iodomethyl)-3-butanol-1-yl-triphosphate (IHPPP); α,γ-di-[3-(bromomethyl)-3-butanol-1-yl]-triphosphate (diBrHTP); and α,γ-di-[3-(iodomethyl)-3-butanol-1-yl]-triphosphate (diIHTP).

In another particular embodiment, the phosphoantigen can be selected from the group consisting of: 3,4-epoxy-3-methyl-1-butyl-diphosphate (Epox-PP); 3,4,-epoxy-3-methyl-1-butyl-triphosphate (Epox-PPP); α,γ-di-3,4,-epoxy-3-methyl-1-butyl-triphosphate (di-Epox-TP); 3,4-epoxy-3-ethyl-1-butyl-diphosphate; 4,5-epoxy-4-methyl-1-pentyl-diphosphate; 4,5-epoxy-4-ethyl-1-pentyl-diphosphate; 5,6-epoxy-5-methyl-1-hexyl-diphosphate; 5,6-epoxy-5-ethyl-1-hexyl-diphosphate; 6,7-epoxy-6-methyl-1-heptyl-diphosphate; 6,7-epoxy-6-ethyl-1-heptyl-diphosphate; 7,8-epoxy-7-methyl-1-octyl-diphosphate; 7,8-epoxy-7-ethyl-1-octyl-diphosphate; 8,9-epoxy-8-methyl-1-nonyl-diphosphate; 8,9-epoxy-8-ethyl-1-nonyl-diphosphate; 9,10-epoxy-9-methyl-1-decyl-diphosphate; 9,10-epoxy-9-ethyl-1-decyl-diphosphate; 10,11-epoxy-10-methyl-1-undecyl-diphosphate; 10,11-epoxy-10-ethyl-1-undecyl-diphosphate; 11,12-epoxy-11-methyl-1-dodecyl-diphosphate; 11,12-epoxy-11-ethyl-1-dodecyl-diphosphate; 12,13-epoxy-12-methyl-1-tridecyl-diphosphate; 12,13-epoxy-12-ethyl-1-tridecyl-diphosphate; 13,14-epoxy-13-methyl-1-tetradecyl-diphosphate; 13,14-epoxy-13-ethyl-1-tetradecyl-diphosphate; 14,15-epoxy-14-methyl-1-pentadecyl-diphosphate; 14,15-epoxy-14-ethyl-1-pentadecyl-diphosphate; 15,16-epoxy-15-methyl-1-hexadecyl-diphosphate; 15,16-epoxy-15-ethyl-1-hexadecyl-diphosphate; 16,17-epoxy-16-methyl-1-heptadecyl-diphosphate; 16,17-epoxy-16-ethyl-1-heptadecyl-diphosphate; 17,18-epoxy-17-methyl-1-octadecyl-diphosphate; 17,18-epoxy-17-ethyl-1-octadecyl-diphosphate; 18,19-epoxy-18-methyl-1-nonadecyl-diphosphate; 18,19-epoxy-18-ethyl-1-nonadecyl-diphosphate; 19,20-epoxy-19-methyl-1-eicosyl-diphosphate; 19,20-epoxy-19-ethyl-1-eicosyl-diphosphate; 20,21-epoxy-20-methyl-1-heneicosyl-diphosphate; 20,21-epoxy-20-ethyl-1-heneicosyl-diphosphate; 21,22-epoxy-21-methyl-1-docosyl-diphosphate; and 21,22-epoxy-21-ethyl-1-docosyl-diphosphate.

In a further particular embodiment, the phosphoantigen can be selected from the group consisting of: 3,4-epoxy-3-methyl-1-butyl-diphosphate (Epox-PP); 3,4,-epoxy-3-methyl-1-butyl-triphosphate (Epox-PPP); α,γ-di-3,4,-epoxy-3-methyl-1-butyl-triphosphate (di-Epox-TP); and uridine 5′-triphosphate-(3,4-epoxy methyl butyl) (Epox-UTP).

In another preferred embodiment, the phosphoantigen can be selected from the group consisting of: (E)-4-hydroxy-3-methyl-2-butenyl pyrophosphate (HDMAPP) and (E)-5-hydroxy-4-methylpent-3-enyl pyrophosphonate (CHDMAPP).

These compounds may be produced according to various techniques known per se in the art, some of which being disclosed in PCT Publications nos. WO 00/12516, WO 00/12519, WO 03/050128, WO 02/083720 and WO 03/009855, the disclosures of which are incorporated herein by reference.

In one preferred embodiment, the phosphoantigen is a γδT cell activator and is a compound described in any one of PCT publication nos. WO 00/12516, WO 00/12519, WO 03/050128, WO 02/083720, WO 03/009855 and WO 05/054258, the disclosures of which Formulas and specific structures as well as synthesis methods are incorporated herein by reference. In another preferred embodiment, the phosphoantigen is a γδT cell activator and is a compound selected from the group consisting of HDMAPP, CHDMAPP, NHDMAPP, H-angelylPP, Epox-PP, BrHPP and CBrHPP.

Alternatively, although less potent in their functions as a γδT cell activator, other activators for use in the present invention are phosphoantigens disclosed in WO 95/20673, isopentenyl pyrophosphate (IPP) (U.S. Pat. No. 5,639,653) and 3-methylbut-3-enyl pyrophosphonate (C-IPP). The disclosures of both references are incorporated herein by reference. Also encompassed are compounds that contain a phosphate moiety and act as γδT cell inhibitors; one example is a compound disclosed in U.S. Pat. No. 6,624,151 B1, the disclosure of which is incorporated herein by reference.

Each of the foregoing references relating to compounds and their synthesis are incorporated herein by reference.

### Step 1: Acidification (Preferably Using a Cationic Resin)

In this first step a phosphoantigen compound is provided in solution, preferably in aqueous or hydroalcoholic solution. Cationic resins that can be used for acidication include generally any strongly acidic cationic resin, for example Dowex 50WX8, available from commercial sources such as Sigma Aldrich.

Step 2: Salification with an Organic Base Solution

The acidified phosphoantigen composition is then salified or neutralized by addition to the composition of an organic base solution. Any suitable organic base can be used, examples include those listed in the section titled “Organic Bases”. In the case of hydrophobic bases it may be necessary to add a co-solvent to the aqueous solution of phosphoantigen prior to this step in order to ensure the homogeneity of the salt solution.

It may be preferable to use a pharmaceutically acceptable organic base such as benzathin in order to simplify the process of manufacturing a composition destined for administration to humans by avoiding downstream exchange of a pharmaceutically unacceptable base with an acceptable one.

### Organic Bases

Several organic bases are described by way of example; it will be appreciated that structurally related compounds may have similar properties and can be used in the same manner, such compounds being within the scope of the invention. Preferred examples of suitable organic bases include natural alkaloids of quinquina, including but not limited to quinine, cinchonidine, cinchonine, and quinidine; quinoline bases, including but not limited to 8-hydroxy-quinoline and 5-chloro-8-hydroxy-quinoline; pharmaceutically acceptable bases including but not limited to benzathin, procain, N-methyl-D-glucamine, diethylamine, ethylenediamine, piperazine, ethanolamine, thiethanolamine, betaine, as well as basic and polar amino-acids, for example lysine and arginine.

### Natural Alkaloids of Quinquina

| | |
|---|---|
| | Formula IV |
| 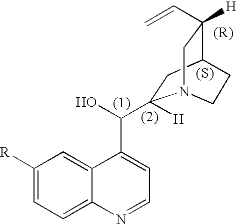 | |

| | R = H | R = MeO |
|---|---|---|
| 1-(R) 2-(S) | Cinchonidine | Quinine |
| 1-(S) 2-(R) | Cinchonine | Quinidine |

In one example, a natural alkaloid of quinquina is used to salify the phosphoantigen compound; preferably said base is selected from the group consisting of quinine, cinchonidine, cinchonine, and quinidine. Preferably a natural alkaloid of quinquina comprises a compound of Formula IV.

### Quinoline Bases

In another preferred example a quinoline base is used to salify the phosphoantigen compound, preferably a base selected from the group consisting of 8-hydroxy-quinoline and 5-chloro-8-hydroxy-quinoline.

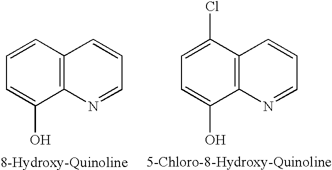

### Pharmaceutically Acceptable Bases

In another preferred example a pharmaceutically acceptable base is used to salify the phosphoantigen compound, preferably a base selected from the group consisting of benzathin, procain, N-methyl-D-glucamine diethylamine, ethylenediamine, piperazine, ethanolamine, thiethanolamine, betaine, as well as basic and polar amino-acids, for example lysine and arginine.

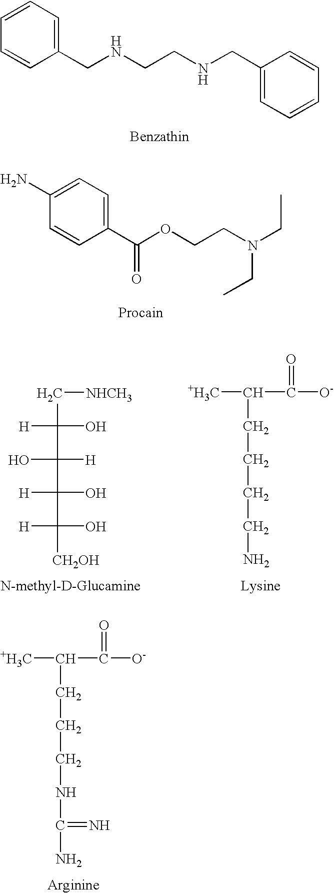

### Step 3: Evaporation of Solvent (Distillation): Solid State Formation (Either Amorphous or Semi-Crystalline).

Evaporation is partial or total, preferably under vacuum with heating. If necessary, this step can be simplified by adding small amounts of a volatile co-solvent thereby decreasing the boiling point of the mixture. If the evaporation is total, then the residue is homogenized in a small amount of a suitable solvent. Suitable solvent includes but is not limited to pharmaceutically acceptable solvents, for example water, ethanol, isopropanol, methanol, acetone, DMSO, ethylene glycol, propylene glycol. If the evaporation is only partial, the crystallization is performed from the concentrated solution obtained.

### Step 4: Crystallization: (First Stage of Crystallization—Formation of a Suspension).

This can be achieved by any of a number of suitable methods, including anti-solvent addition, slow cooling, solvent evaporation or any combination thereof. Alternatively and preferably the solution can be cooled slowly, or solvent is slowly evaporated. In general, the most preferred method used is antisolvent addition; next preferred methods in decreasing order of preference are (i) slow cooling and (ii) slow evaporation of solvent in a controlled atmosphere.

### Step 4′: Slurry Maturation:

This step is optional but is preferably carried out in order to improve—the crystallinity (quality of the crystallographic long range order) and the structural purity (a single phase only exists e.g. no concomitant polymorphs or various co-existing solvates). The step involves subjecting the suspension of step 4 to maturation cycle(s) before solid isolation in step 5, a cycle generally comprising a fast heating step followed by a slow cooling step.

### Step 5: Crystallized Solid Isolation.

This step is advantageously carried out by filtration or centrifugation of the suspension.

### Step 5′: Optional Washings or Slurries.

This step is optional but can be advantageous. The residual filtrate of step 5 trapped in the solid cake is eliminated, which will generally results in an improvement in purity and faster drying in certain cases (e.g. acetone washing) depending on the nature of the particular solvent used.

### Step 6: Drying of the Crystallized Solid

The crystal of step 5 or 5′ is dried under suitable conditions, care being taken so as not to denature the solid phase. Temperatures and conditions can be determined based on the properties of the solid; however, drying at ambient atmosphere is expected to be generally applicable.

### Step 7: Recrystallization

While step 7 is optional, it is highly preferable to recrystallize in several successive cycles in order to improve the purity of the composition. The crystallized solid of step 6 is homogenized in a small amount of a suitable solvent in step 3 and the remainder of the steps (i.e., step 4 and onwards) are carried out. This may improve the purity and the crystallinity of the solid phase. Recrystallization can be carried out any number of times, but preferably at least 1, 2, 3 or 4 cycles of recrystallization are carried out prior to solid formulation.

### Optional: Obtaining Compositions for Pharmaceutical Use

In cases where the organic base used in the crystallization process is not pharmaceutically acceptable, or whether a different base is desired by any other reason, the purified phosphoantigen salt can be converted to any other suitable pharmaceutically acceptable salt using cation exchange techniques substantially as described in step (1) above, for pharmaceutical formulation or development. Salts of pharmaceutically acceptable bases such as benzathine, however, can be used directly in drug product formulations, making this step unnecessary.

The resulting composition can thereafter be further formulated according to any suitable method, several examples are provided in the section titled “Pharmaceuticals”.

### Novel Crystalline Phases

One embodiment of the invention encompasses a crystalline solvate of a phosphoantigen salt, particularly a crystalline solvate of a phosphoantigen quinine salt, a crystalline solvate of a phosphoantigen cinchonidine salt, a crystalline solvate of a phosphoantigen 8-hydroxyquinoline salt and a crystalline solvate of a phosphoantigen benzathine salt.

Another embodiment of the invention encompasses a crystalline salt of a compound of Formula I to III, particularly a crystalline quinine salt, a cinchonidine salt, an 8-hydroxyquinoline salt and a benzathine salt of a compound of Formula I to III.

In a further embodiment, the invention encompasses a crystalline BrHPP, IPP, HDMAPP, C-HDMAPP, N-HDMAPP and H-angelylPP salt, particularly a crystalline quinine salt, a cinchonidine salt, an 8-hydroxyquinoline salt and a benzathine salt of BrHPP, IPP, HDMAPP, C-HDMAPP, N-HDMAPP and H-angelylPP.

In yet other embodiments, the invention encompasses a crystalline solvate of a bisphosphonate, a nucleotide, nucleotide analog or phosphate-comprising nucleoside or nucleoside analog. Particularly preferred is a crystalline solvate of a quinine salt, a crystalline solvate of a cinchonidine salt, a crystalline solvate of a 8-hydroxyquinoline salt and a crystalline solvate of a benzathine salt of a bisphosphonate, a nucleotide, nucleotide analog or phosphate-comprising nucleoside or nucleoside analog.

### Crystalline Phases A to O

In a further preferred embodiment, disclosed are a series of crystalline phases of phosphoantigen salts, designated Phases A to O herein. XRPD pattern peaks are expressed in terms of 2-theta angles, said patterns were obtained according to the procedures mentioned below; it will be appreciated that due to variation in measurement a peak is expected to vary by +/−0.02° 2-theta. Where “about” is used in connection with a 2-theta angle, this preferably means the number +/−0.02.

Phase A: BrHPP Quinine Salts, Crystalline (n,p)-Mix-I

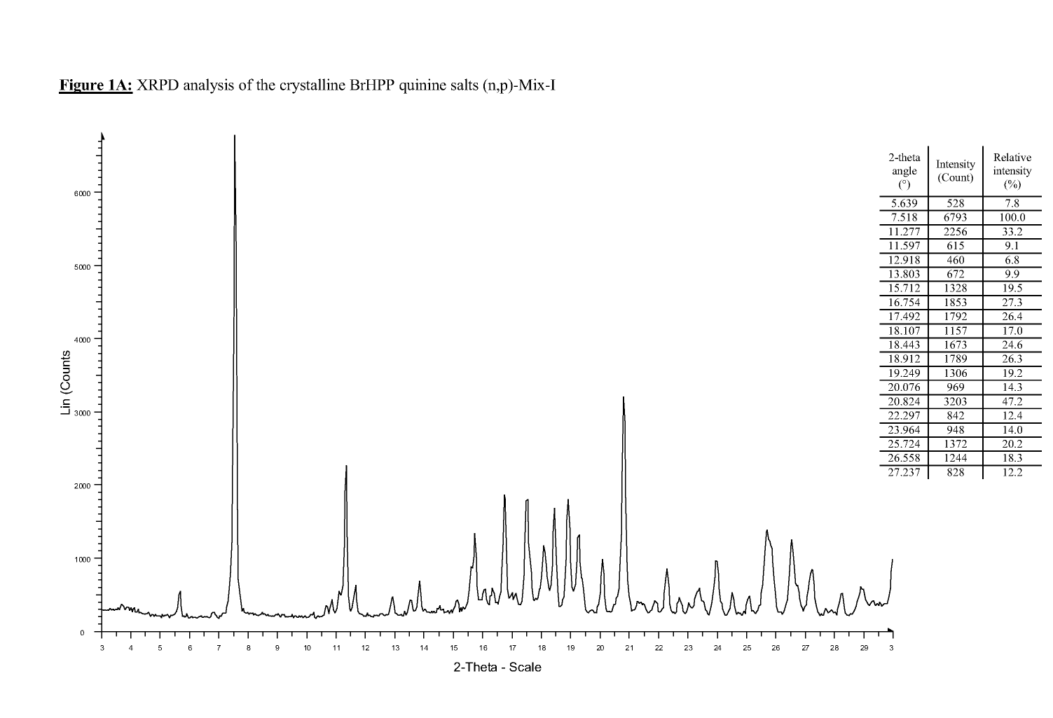

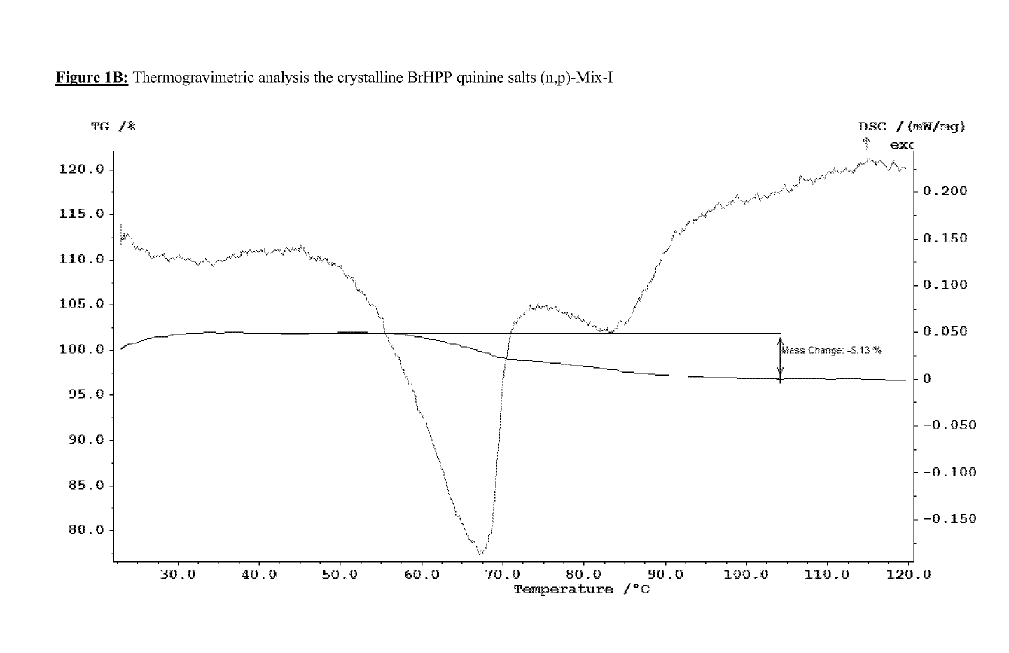

A first novel crystalline phase of BrHPP is Phase A, characterized by a powder X-ray diffraction (XRPD) pattern comprising significant peaks (2-theta angles) at about 5.64, 7.52, 11.28, 11.60, 12.92, 13.80, 15.71, 16.75, 17.49, 18.11, 18.44, 18.91, 19.25, 20.08, 20.82, 22.30, 23.96, 25.72, 26.56 and 27.24 degrees 2-theta. Preferably, the phase is characterised by an XRPD pattern comprising at least one, two, three, four or all of the above peaks (2-theta angles), more particularly selected in the group consisting of the peaks at about 7.52, 11.28, 16.75, 18.91 and 20.82. The crystalline phase can also be characterised as having a powder X-ray diffraction pattern substantially as depicted in FIG. 1A. A combination of any two, any three, any four, any five, or any six (or more) of the above peaks or of those in FIG. 1A can be used to characterize BrHPP Quinine salts, crystalline (n,p) Mix-I. A process for preparing BrHPP Quinine salts, crystalline (n,p)-Mix-I is shown in Example 1.

Phase B: BrHPP Quinine Salts, Crystalline (n,p)-Mix-II

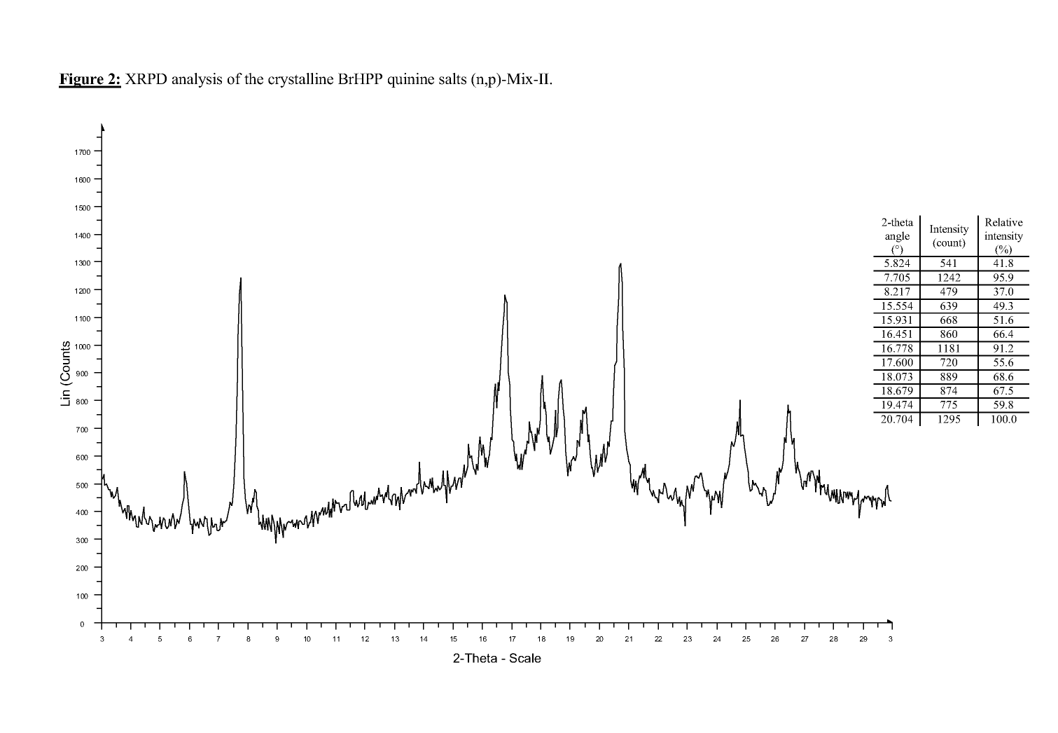

Another crystalline phase of BrHPP is characterized by a powder X-ray diffraction pattern comprising significant peaks (2-theta angles) at about 5.82, 7.71, 8.22, 15.55, 15.93, 16.45, 16.78, 17.60, 18.07, 18.68, 19.47 and 20.70 degrees 2-theta. Preferably, the phase is characterised by an XRPD pattern comprising at least one, two, three, four or all of the above peaks (2-theta angles), more particularly selected in the group consisting of the peaks at about 7.71, 16.78 and 20.70. In another embodiment, the invention provides a crystalline phase characterised by a powder X-ray diffraction pattern substantially as depicted in FIG. 2. A combination of any two, any three, any four, any five, or any six (or more) of the above peaks or of those in FIG. 2 can be used to characterize BrHPP Quinine salts, crystalline (n,p) Mix-II. A process for preparing BrHPP Quinine salts, crystalline (n,p)-Mix-II is shown in Example 1.

Phase C: BrHPP Quinine Salts, Crystalline (n,p)-Mix-III

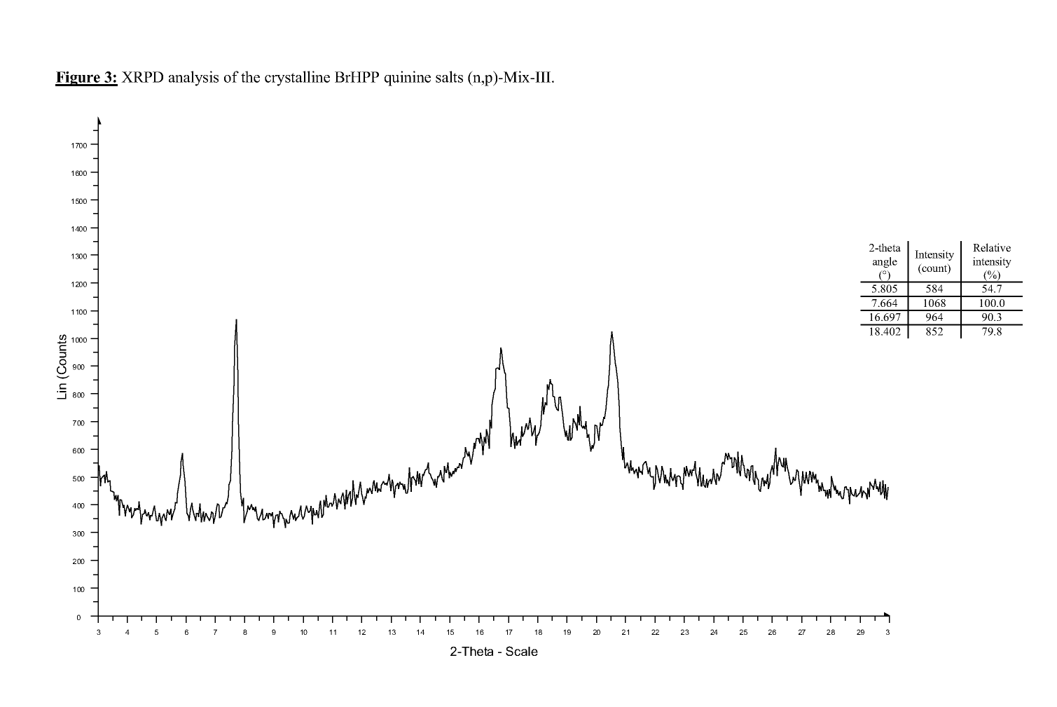

Another crystalline phase of BrHPP is characterized by a powder X-ray diffraction pattern comprising significant peaks (2-theta angles) at about 5.81, 7.66, 16.70 and 18.40 degrees 2-theta. Preferably, the phase is characterised by an XRPD pattern comprising at least one, two, three, four or all of the above peaks (2-theta angles), more particularly selected in the group consisting of the peaks at about 7.66, 16.70 and 18.40. In another embodiment, the invention provides a crystalline phase characterised by a powder X-ray diffraction pattern substantially as depicted in FIG. 3. A combination of any two, any three, any four, any five, or any six (or more) of the above peaks or of those in FIG. 3 can be used to characterize BrHPP Quinine salts, crystalline (n,p) Mix-III. A process for preparing BrHPP Quinine salts, crystalline (n,p)-Mix-III is shown in Example 1.

Phase D: BrHPP Cinchonidine Salts, Crystalline (n,p)-Mix-I

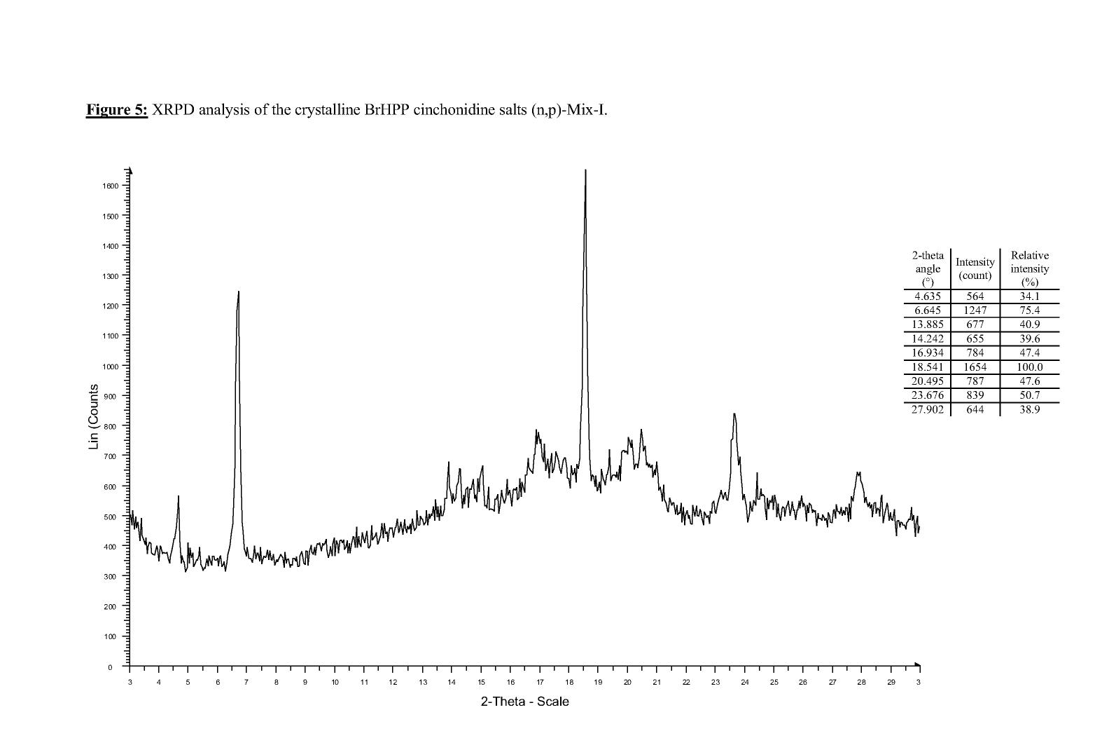

Another crystalline phase of BrHPP is characterized by a powder X-ray diffraction pattern comprising significant peaks (2-theta angles) at about 4.64, 6.65, 13.89, 14.24, 16.93, 18.54, 20.50, 23.68 and 27.90 degrees 2-theta. Preferably, the phase is characterised by an XRPD pattern comprising at least one, two, three, four or all of the above peaks (2-theta angles), more particularly selected in the group consisting of the peaks at about 6.65, 18.54 and 23.68. In another embodiment, the invention provides a crystalline phase characterised by a powder X-ray diffraction pattern substantially as depicted in FIG. 5. A combination of any two, three, four, any five or any six (or more) of the above peaks or of those in FIG. 5 can be used to characterize BrHPP cinchonidine salts, crystalline (n,p)-Mix-I. A process for preparing BrHPP cinchonidine salts, crystalline (n,p)-Mix-I is shown in Example 2.

Phase E: BrHPP Cinchonidine Salts, Crystalline (n,p)-Mix-II

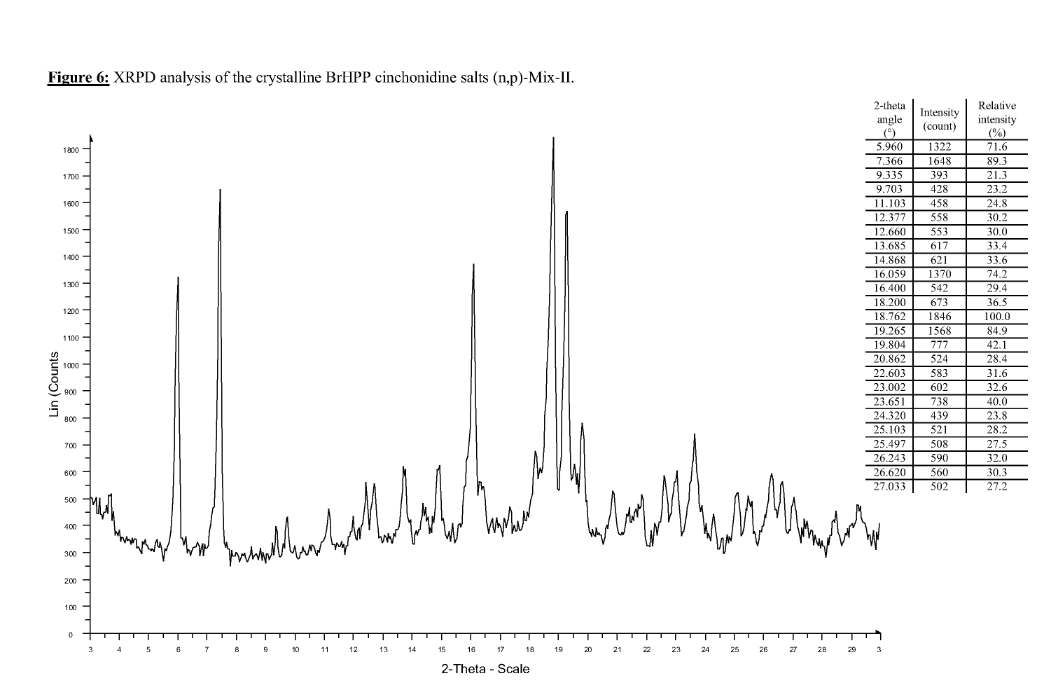

Another crystalline phase of BrHPP is characterized by a powder X-ray diffraction pattern comprising significant peaks (2-theta angles) at about 5.96, 7.37, 9.34, 9.70, 11.10, 12.38, 12.66, 13.69, 14.87, 16.06, 16.40, 18.20, 18.76, 19.27, 19.80, 20.86, 22.60, 23.00, 23.65, 24.32, 25.10, 25.50, 26.24, 26.62 and 27.03 degrees 2-theta. Preferably, the phase is characterised by an XRPD pattern comprising at least one, two, three, four or all of the above peaks (2-theta angles), more particularly selected in the group consisting of the peaks at about 5.96, 7.37, 16.06 and 19.27. In another embodiment, the invention provides a crystalline phase characterised by a powder X-ray diffraction pattern substantially as depicted in FIG. 6. A combination of any two, any three, any four, any five, or any six (or more) of the above peaks or of those in FIG. 6 can be used to characterize BrHPP cinchonidine salts, crystalline (n,p)-Mix-II. A process for preparing BrHPP cinchonidine salts, crystalline (n,p)-Mix-II is shown in Example 2.

### Phase F: BrHPP 8-Hydroxyquinoline Salts, Crystalline Phase-I

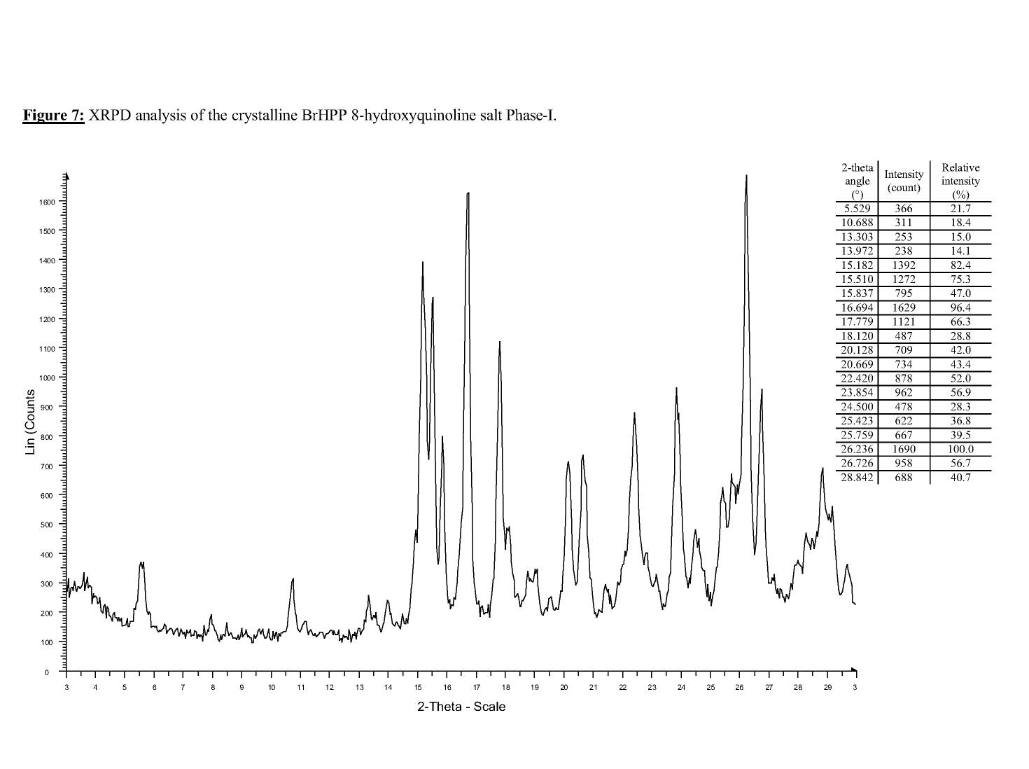

Another crystalline phase of BrHPP is characterized by a powder X-ray diffraction pattern comprising significant peaks (2-theta angles) at about 5.53, 10.69, 13.30, 13.97, 15.18, 15.51, 15.84, 16.69, 17.78, 18.12, 20.13, 20.67, 22.42, 23.85, 24.50, 25.42, 25.76, 26.24, 26.73 and 28.84 degrees 2-theta. Preferably, the phase is characterised by an XRPD pattern comprising at least one, two, three, four or all of the above peaks (2-theta angles), more particularly selected in the group consisting of the peaks at about 15.18, 15.51, 16.69, 17.78 and 26.24. In another embodiment, the invention provides a crystalline phase characterised by a powder X-ray diffraction pattern substantially as depicted in FIG. 7. A combination of any two, any three, any four, any five, or any six (or more) of the above peaks or of those in FIG. 7 can be used to characterize BrHPP 8-hydroxyquinoline salts, crystalline Phase-I. A process for preparing BrHPP 8-hydroxyquinoline salts, crystalline Phase-I is shown in Example 3.

### Phase G: BrHPP Benzathin Salt (Rac)-Mix-I

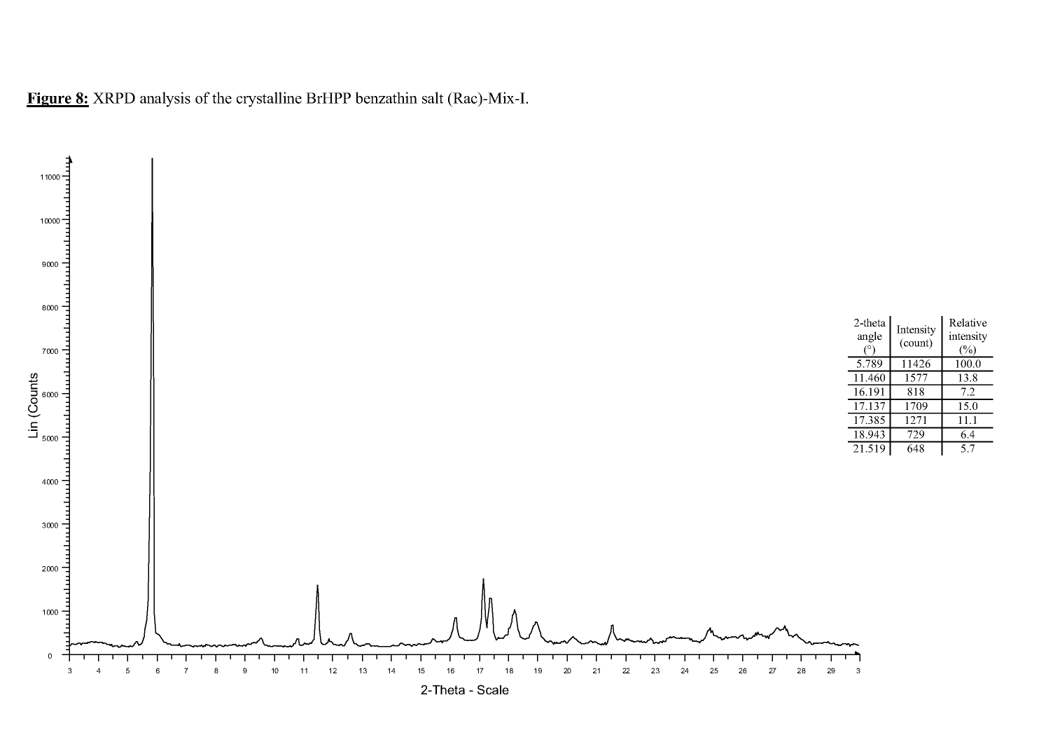

Another crystalline phase of BrHPP is characterized by a powder X-ray diffraction pattern comprising significant peaks (2-theta angles) at about 5.79, 11.46, 16.19, 17.14, 17.39, 18.94 and 21.52 degrees 2-theta. Preferably, the phase is characterised by an XRPD pattern comprising at least one, two, three, four or all of the above peaks (2-theta angles), more particularly selected in the group consisting of the peaks at about 5.79, 11.46 and 17.14. In another embodiment, the invention provides a crystalline phase characterised by a powder X-ray diffraction pattern substantially as depicted in FIG. 8. A combination of any two, any three, any four, any five, or any six (or more) of the above peaks or of those in FIG. 8 can be used to characterize BrHPP benzathin salt (Rac)-Mix-I. A process for preparing BrHPP benzathin salt (Rac)-Mix-I is shown in Example 4.

### Phase H: (E)-C-HDMAPP Quinine Salts, Crystalline (E)-Phase-I

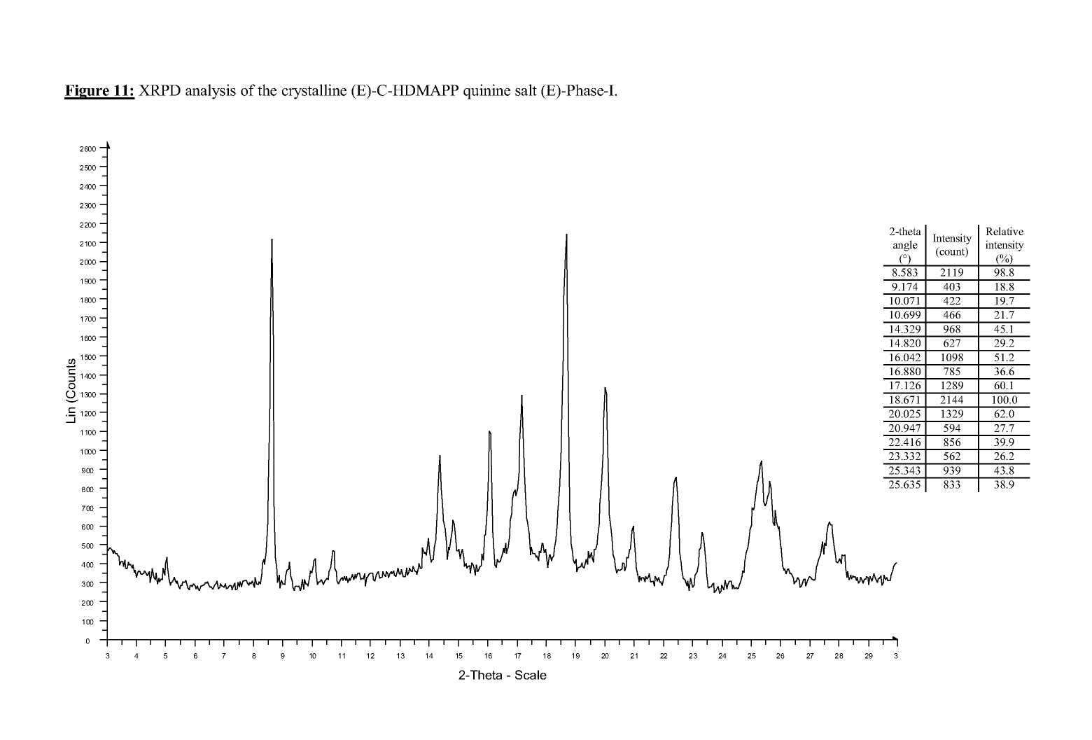

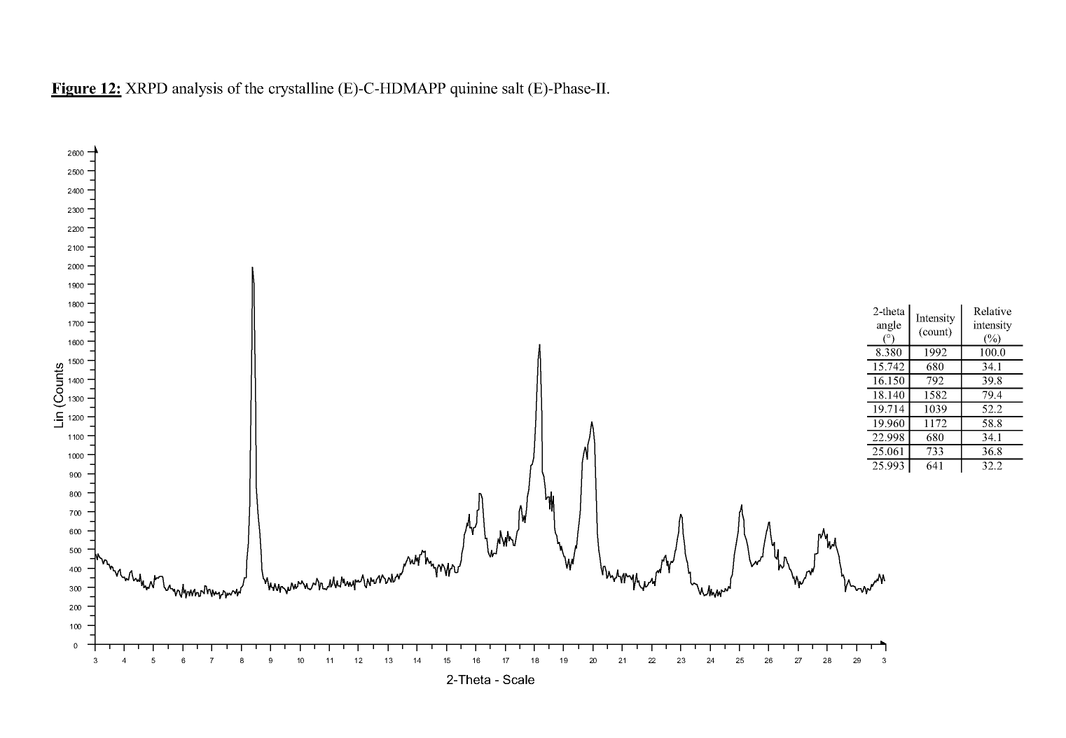

Also disclosed are crystalline phases of C-HDMAPP. In one example a crystalline phase of (E)-C-HDMAPP is characterized by a powder X-ray diffraction pattern comprising significant peaks (2-theta angles) at about 8.58, 9.17, 10.07, 10.70, 14.33, 14.82, 16.04, 16.88, 17.13, 18.67, 20.03, 20.95, 22.42, 23.33, 25.34 and 25.64 degrees 2-theta. Preferably, the phase is characterised by an XRPD pattern comprising at least one, two, three, four or all of the above peaks (2-theta angles), more particularly selected in the group consisting of the peaks at about 8.58, 17.13, 18.67 and 20.03. In another embodiment, the invention provides a crystalline phase characterised by a powder X-ray diffraction pattern substantially as depicted in FIG. 11. A combination of any two, any three, any four, any five, or any six (or more) of the above peaks or of those in FIG. 11 can be used to characterize (E)-C-HDMAPP quinine salts, crystalline (E)-Phase-I. A process for preparing (E)-C-HDMAPP quinine salts, crystalline (E)-Phase-I is shown in Example 5.

### Phase I: (E)-C-HDMAPP Quinine Salts, Crystalline (E)-Phase-II

Another embodiment encompasses a crystalline phase of (E)-C-HDMAPP characterized by a powder X-ray diffraction pattern comprising significant peaks (2-theta angles) at about 8.38, 15.74, 16.15, 18.14, 19.71, 19.96, 23.00, 25.06 and 25.99 degrees 2-theta. Preferably, the phase is characterised by an XRPD pattern comprising at least one, two, three, four or all of the above peaks (2-theta angles), more particularly selected in the group consisting of the peaks at about 8.38, 18.14, 19.71 and 19.96. In another embodiment, the invention provides a crystalline phase characterised by a powder X-ray diffraction pattern substantially as depicted in FIG. 12. A combination of any two, any three, any four, any five, or any six (or more) of the above peaks or of those in FIG. 12 can be used to characterize (E)-C-HDMAPP quinine salts, crystalline (E)-Phase-II. A process for preparing (E)-C-HDMAPP quinine salts, crystalline (E)-Phase-II is shown in Example 5.

### Phase J: (E)-C-HDMAPP Benzathin Salt, Crystalline (E)-Phase-I

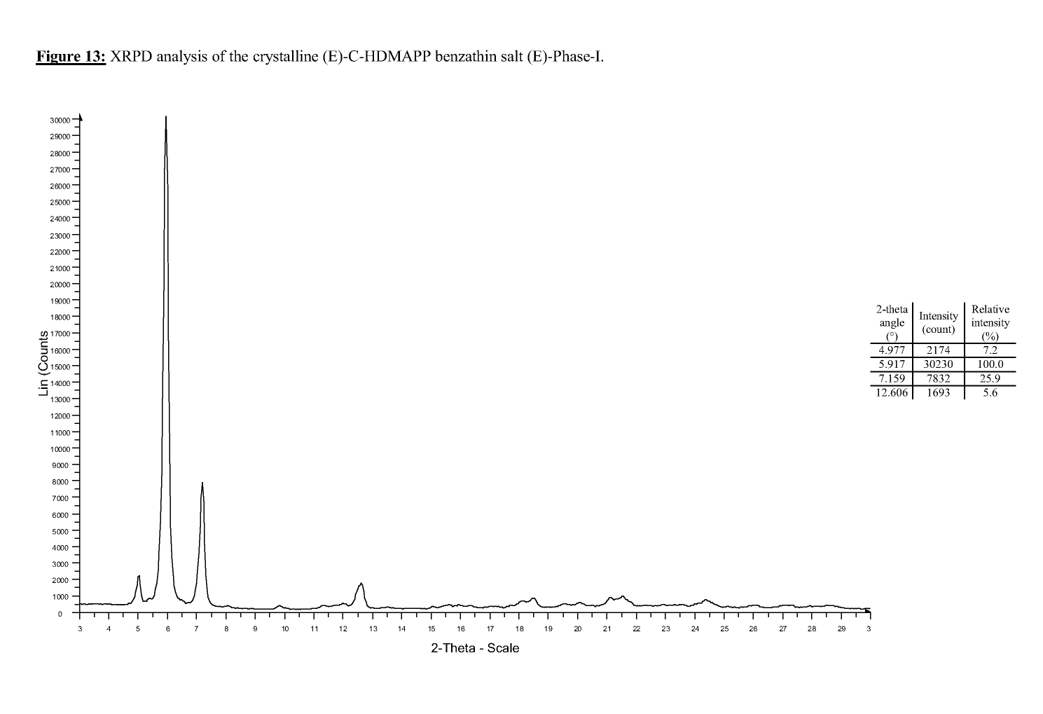

Another embodiment encompasses a crystalline phase of (E)-C-HDMAPP characterized by a powder X-ray diffraction pattern comprising significant peaks (2-theta angles) at about 4.98, 5.92, 7.16 and 12.61 degrees 2-theta. Preferably, the phase is characterised by an XRPD pattern comprising at least one, two, three, four or all of the above peaks (2-theta angles), more particularly selected in the group consisting of the peaks at about 5.92 and 7.16. In another embodiment, the invention provides a crystalline phase characterised by a powder X-ray diffraction pattern substantially as depicted in FIG. 13. A combination of any two, any three, any four, any five, or any six (or more) of the above peaks or of those in FIG. 13 can be used to characterize (E)-C-HDMAPP benzathin salt, crystalline (E)-Phase-I. A process for preparing (E)-C-HDMAPP benzathin salt, crystalline (E)-Phase-I is shown in Example 6.

Phase K: (E)-C-HDMAPP Benzathin salt, Crystalline (E)-Phase-II

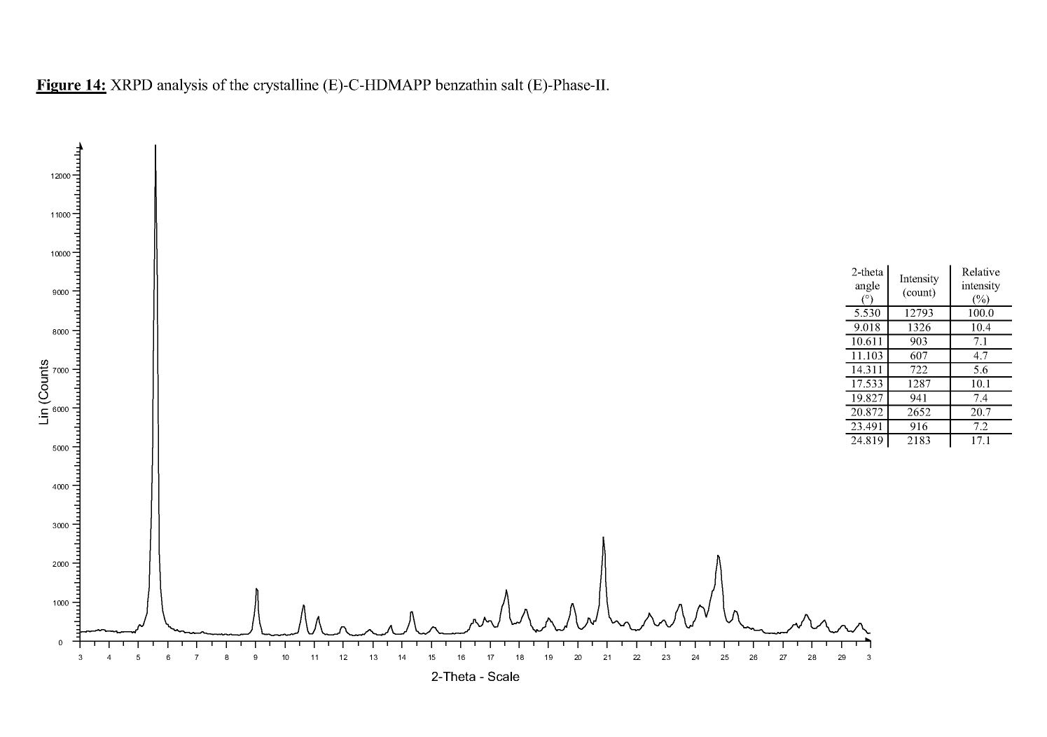

Another embodiment encompasses a crystalline phase of (E)-C-HDMAPP characterized by a powder X-ray diffraction pattern comprising significant peaks (2-theta angles) at about 5.53, 9.02, 10.61, 11.10, 14.31, 17.53, 19.83, 20.87, 23.49 and 24.82 degrees 2-theta. Preferably, the phase is characterised by an XRPD pattern comprising at least one, two, three, four or all of the above peaks (2-theta angles), more particularly selected in the group consisting of the peaks at about 5.53, 20.87, and 24.82. In another embodiment, the invention provides a crystalline phase characterised by a powder X-ray diffraction pattern substantially as depicted in FIG. 14. A combination of any two, any three, any four, any five, or any six (or more) of the above peaks or of those in FIG. 14 can be used to characterize (E)-C-HDMAPP benzathin salt, crystalline (E)-Phase-II. A process for preparing (E)-C-HDMAPP benzathin salt, crystalline (E)-Phase-II is shown in Example 6.

### Phase L: IPP Quinine Salt, Crystalline Phase-I

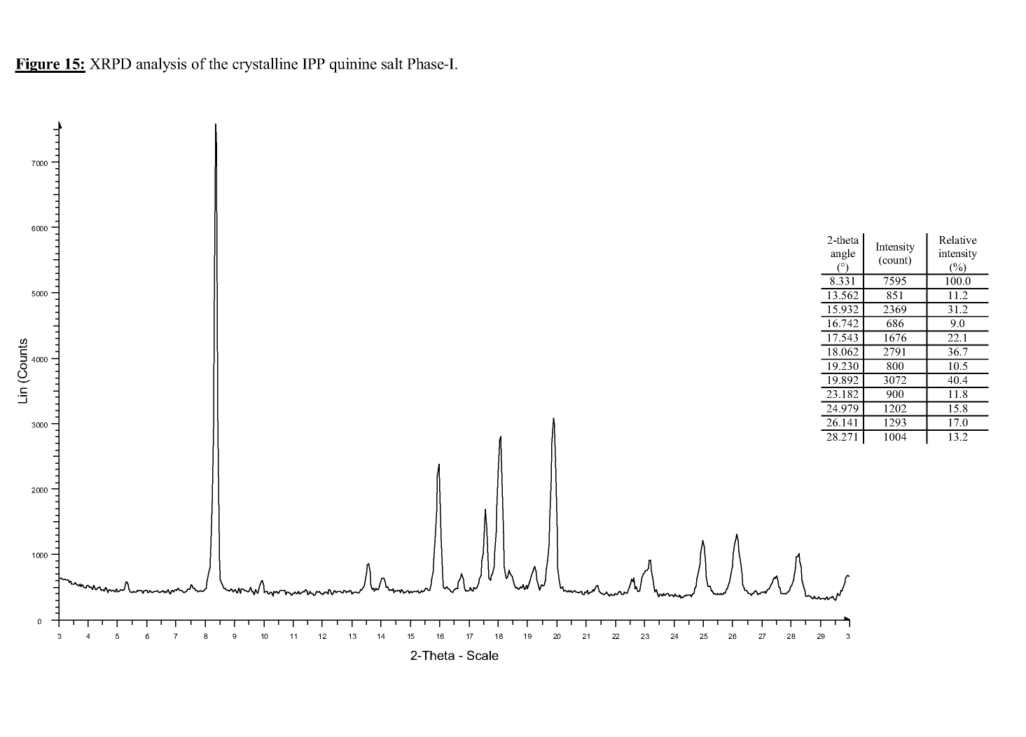

Also disclosed are crystalline phases of IPP. In one example a crystalline phase of IPP is characterized by a powder X-ray diffraction pattern comprising significant peaks (2-theta angles) at about 8.33, 13.56, 15.93, 16.74, 17.54, 18.06, 19.23, 19.89, 23.18, 24.98, 26.14 and 28.27 degrees 2-theta. Preferably, the phase is characterised by an XRPD pattern comprising at least one, two, three, four or all of the above peaks (2-theta angles), more particularly selected in the group consisting of the peaks at about 8.33, 15.93, 18.06 and 19.89. In another embodiment, the invention provides a crystalline phase characterised by a powder X-ray diffraction pattern substantially as depicted in FIG. 15. A combination of any two, any three, any four, any five, or any six (or more) of the above peaks or of those in FIG. 15 can be used to characterize IPP quinine salt, crystalline Phase-I. A process for preparing IPP quinine salt, crystalline Phase-I is shown in Example 7.

### Phase M: IPP Quinine Salt, Crystalline Phase-II

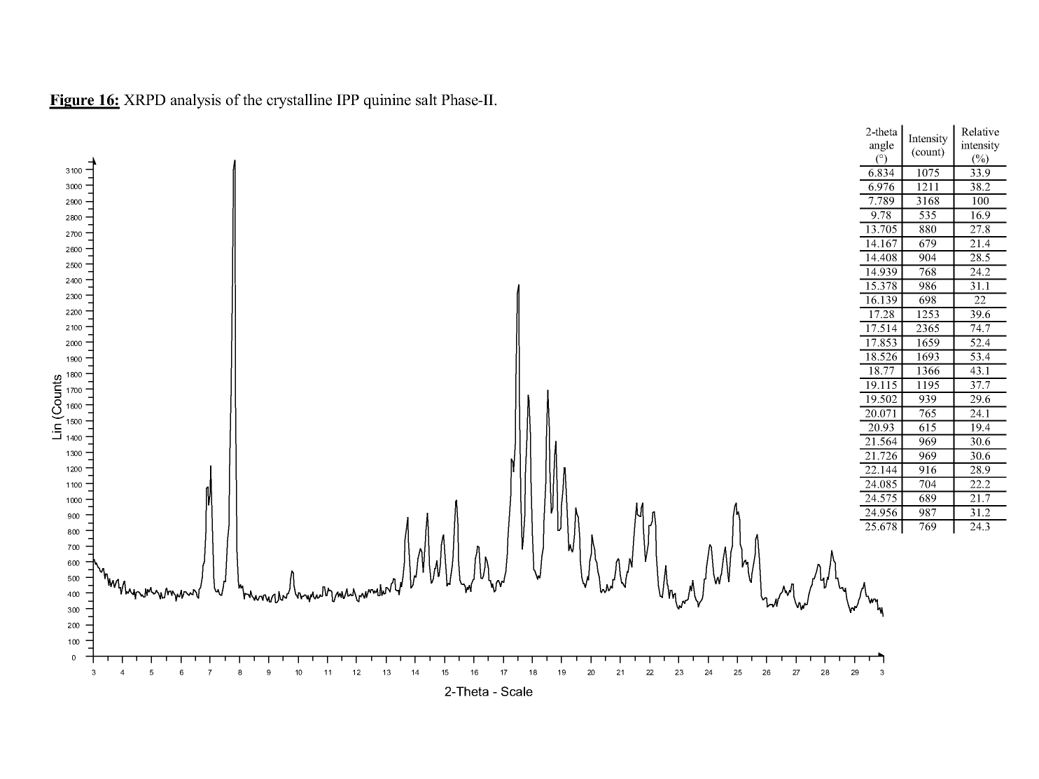

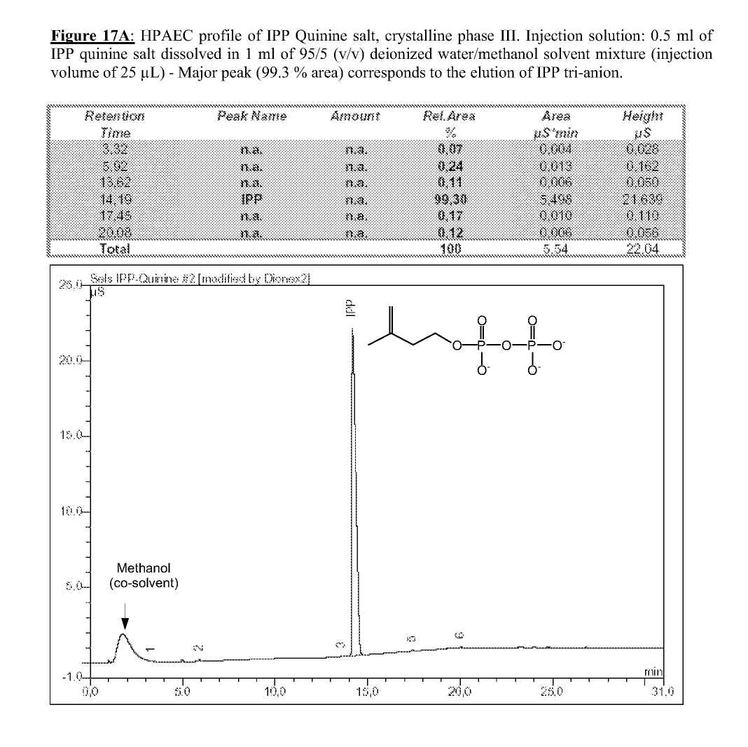

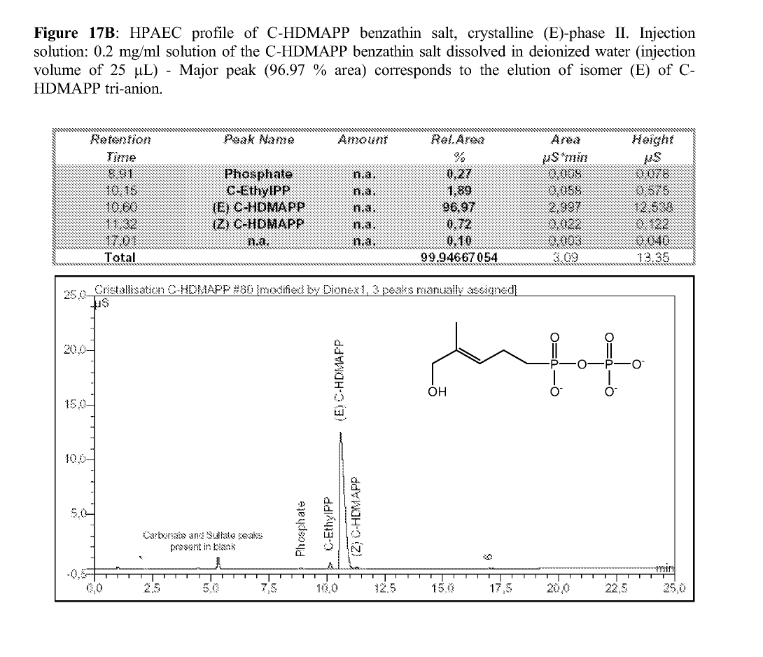

Another embodiment encompasses a crystalline phase of IPP characterized by a powder X-ray diffraction pattern comprising significant peaks (2-theta angles) at about 6.83, 6.98, 7.79, 9.78, 13.71, 14.17, 14.41, 14.94, 15.38, 16.14, 17.28, 17.51, 17.85, 18.53, 18.77, 19.12, 19.50, 20.07, 20.93, 21.56, 21.73, 22.14, 24.09, 24.58, 24.96 and 25.68 degrees 2-theta. Preferably, the phase is characterised by an XRPD pattern comprising at least one, two, three, four or all of the above peaks (2-theta angles), more particularly selected in the group consisting of the peaks at about 7.79, 17.51, 17.85 and 18.53. In another embodiment, the invention provides a crystalline phase characterised by a powder X-ray diffraction pattern substantially as depicted in FIG. 16. A combination of any two, any three, any four, any five, or any six (or more) of the above peaks or of those in FIG. 16 can be used to characterize IPP quinine salt, crystalline Phase-II. A process for preparing IPP quinine salt, crystalline Phase-II is shown in Example 7.

### Phase N: BrHPP Benzathin Salt (Rac)-Mix-II

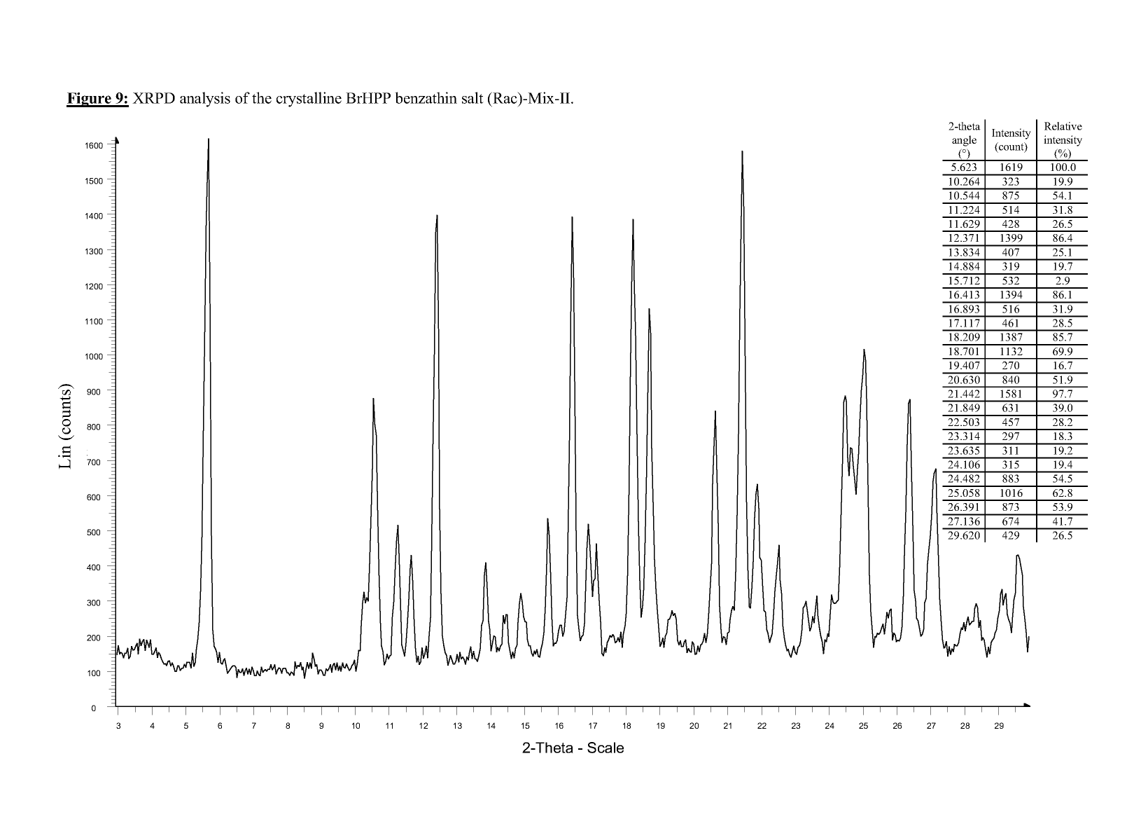

Another crystalline phase of BrHPP is characterized by a powder X-ray diffraction pattern comprising significant peaks (2-theta angles) at about 5.62, 10.26, 10.54, 11.22, 11.63, 12.37, 13.83, 14.88, 15.72, 16.41, 16.89, 17.12, 18.21, 18.70, 19.41, 20.63, 21.44, 21.85, 22.50, 23.31, 23.64, 24.11, 24.48, 25.06, 26.39, 27.14 and 29.62 degrees 2-theta. Preferably, the phase is characterised by an XRPD pattern comprising at least one, two, three, four or all of the above peaks (2-theta angles), more particularly selected in the group consisting of the peaks at about 5.62, 12.37, 16.41, 18.21, 18.70, 21.44, 25.06. In another embodiment, the invention provides a crystalline phase characterised by a powder X-ray diffraction pattern substantially as depicted in FIG. 9. A combination of any two, any three, any four, any five, or any six (or more) of the above peaks or of those in FIG. 9 can be used to characterize BrHPP benzathin salt (Rac)-Mix-II. A process for preparing BrHPP benzathin salt (Rac)-Mix-II is shown in Example 4.

### Phase O: BrHPP Benzathin Salt (Rac)-Mix-III

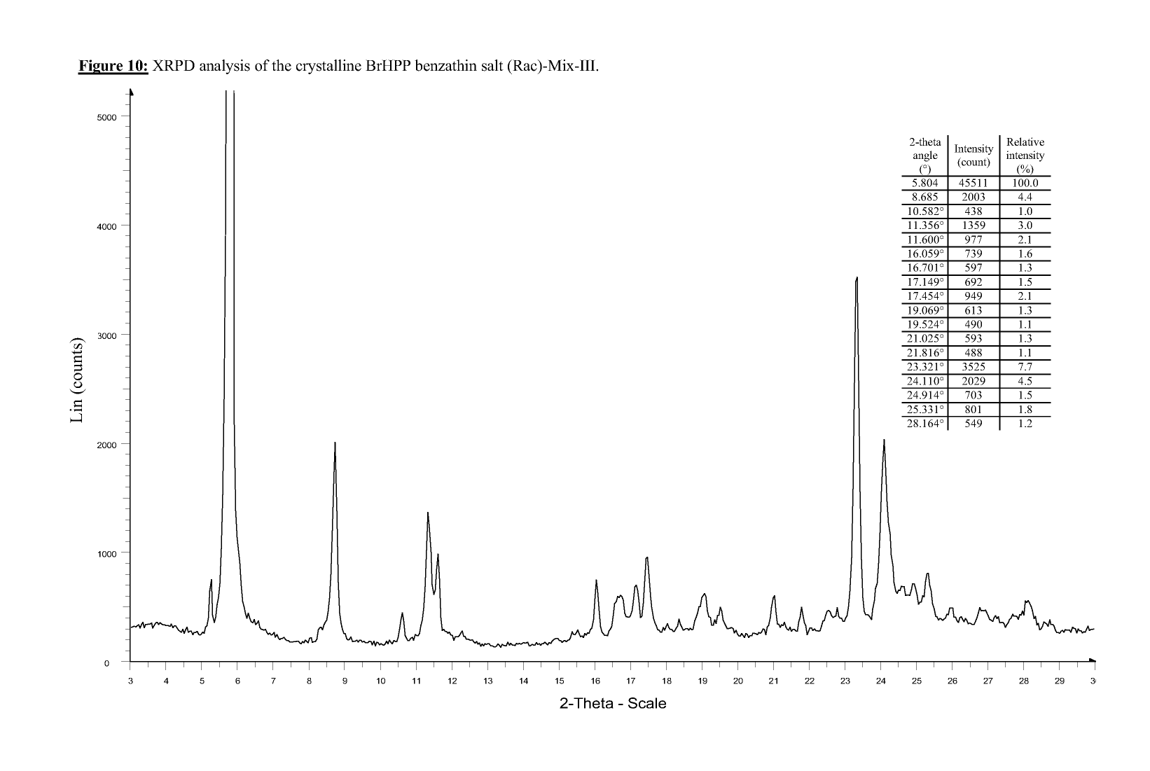

Another crystalline phase of BrHPP is characterized by a powder X-ray diffraction pattern comprising significant peaks (2-theta angles) at about 5.80, 8.68, 10.58, 11.36, 11.60, 16.06, 16.70, 17.15, 17.45, 19.07, 19.52, 21.02, 21.82, 23.32, 24.11, 24.91, 25.33 and 28.16 degrees 2-theta. Preferably, the phase is characterised by an XRPD pattern comprising at least one, two, three, four or all of the above peaks (2-theta angles), more particularly selected in the group consisting of the peaks at about 5.80, 8.68, 11.36, 23.32, 24.11. In another embodiment, the invention provides a crystalline phase characterised by a powder X-ray diffraction pattern substantially as depicted in FIG. 10. A combination of any two, any three, any four, any five, or any six (or more) of the above peaks or of those in FIG. 10 can be used to characterize BrHPP benzathin salt (Rac)-Mix-III. A process for preparing BrHPP benzathin salt (Rac)-Mix-III is shown in Example 4.

The general method provided herein can be used to prepare any of the above crystalline phases. Said process for preparing the crystalline phase comprises the steps of dissolving the phosphoantigen in an aqueous solution and exchanging the cation in the presence of the base solvate, thereby forming a salt with the base and precipitating the crystalline form, preferably a quinine salt, cinchonidine salt, 8-hydroxyquinoline salt or benzathine salt; and isolating the crystals, preferably by evaporating the solvent. The process may further comprise cooling the solution prior to isolating the crystals.

The crystalline phase of phosphoantigen may be a dehydrate, anhydrate, hemihydrate, or dihydrate, solvated and/or hydrated with various stoichiometries.

Yet another embodiment of the invention encompasses a pharmaceutical composition comprising at least one crystalline phase of phosphoantigen selected from the group consisting of phases A to O.

### Pharmaceuticals

A further embodiment of the present invention provides a pharmaceutical composition comprising a phosphoantigen according to the invention, preferably a high purity, stable and/or non-hygroscopic phosphoantigen. Preferably the phosphoantigen drug substance used to prepare the pharmaceutical is a crystalline phosphoantigen, including but limited to one or more of the crystalline phases A to O, or a mixture thereof, in a detectable amount. By “drug substance” is meant the active pharmaceutical ingredient (API). The amount of crystalline phases of a phosphoantigen—for example including but not limited to any one of crystalline Phases A to O or mixture thereof—in the drug substance can be quantified by the use of physical methods such as X-ray powder diffraction (XRPD), solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy.

In a class of this embodiment, about 5% to about 100% by weight of the crystalline phosphoantigen is present in the drug substance. In a second class of this embodiment, about 10% to about 100% by weight of the crystalline phosphoantigen or mixture thereof is present in the drug substance. In a third class of this embodiment, about 25% to about 100% by weight of the crystalline phosphoantigen or mixture thereof is present in the drug substance. In a fourth class of this embodiment, about 50% to about 100% by weight of the crystalline phosphoantigen or mixture thereof is present in the drug substance. In a fifth class of this embodiment, about 75% to about 100% by weight of the crystalline phosphoantigen or mixture thereof is present in the drug substance. In a sixth class of this embodiment, substantially all of the phosphoantigen drug substance is the crystalline phosphoantigen or mixture thereof, i.e., the phosphoantigen drug substance is substantially or essentially pure phosphoantigen or a mixture thereof. Most preferably said phosphoantigen is selected from the group consisting of a compound of Formulas I to III, or a mixture thereof. In the framework of the present invention, the expression “Formulas I to III”, designate all compounds derived from Formulas I to III: I, II IIa, III, IIIa, IIIa1, IIIa2, IIIa3, A, B, IIIb, IIIb1, IIIb2, IIIb3, C, IIIc, IIIc1, IIIc2, IIIc3, D, E, F and G.

The present invention also provides for the use of a phosphoantigen of the present invention in the manufacture of a medicament for the prevention or treatment of clinical conditions for which phosphoantigens are indicated or useful, for example in the treatment of cancer, for immunomodulatory purposes, for infectious disease treatment and/or prevention, for treatment of autoimmune disorders, for osteoporosis, osteitis deformans (“Paget's disease of bone”), bone metastasis (with or without hypercalcemia), multiple myeloma and other conditions that feature bone fragility.

The present invention also provides pharmaceutical compositions comprising the crystalline phosphoantigen, in association with one or more pharmaceutically acceptable carriers or excipients. In one embodiment the pharmaceutical composition comprises a prophylactically or therapeutically effective amount of the active pharmaceutical ingredient (API) in admixture with pharmaceutically acceptable excipients wherein the API comprises a detectable amount of the crystalline phosphoantigen of the present invention. In a second embodiment the pharmaceutical composition comprises a prophylactically or therapeutically effective amount of the API in admixture with pharmaceutically acceptable excipients wherein the API comprises about 5% to about 100% by weight of the crystalline phosphoantigen of the present invention. In a class of this second embodiment, the API in such compositions comprises about 10% to about 100% by weight of the crystalline phosphoantigen. In a second class of this embodiment, the API in such compositions comprises about 25% to about 100% by weight of the crystalline phosphoantigen. In a third class of this embodiment, the API in such compositions comprises about 50% to about 100% by weight of the crystalline phosphoantigen. In a fourth class of this embodiment, the API in such compositions comprises about 75% to about 100% by weight of the crystalline phosphoantigen. In a fifth class of this embodiment, substantially all of the API is the crystalline phosphoantigen, i.e., the API is substantially or essentially pure phosphoantigen or a mixture thereof.

The compositions in accordance with the invention are suitably in unit dosage forms such as tablets, pills, capsules, powders, granules, sterile solutions or suspensions, metered aerosol or liquid sprays, drops, ampoules, auto-injector devices or suppositories. The compositions are intended for oral, parenteral, intranasal, sublingual, or rectal administration, or for administration by inhalation or insufflation. Formulation of the compositions according to the invention can conveniently be effected by methods known from the art, for example, as described in Remington's Pharmaceutical Sciences, 18^{th} ed., Mack Publishing, Easton, Pa. (1990).

The dosage regimen is selected in accordance with a variety of factors including type, species, age, weight, sex and medical condition of the patient; the severity of the condition to be treated; the route of administration; and the renal and hepatic function of the patient. An ordinarily skilled physician, veterinarian, or clinician can readily determine and prescribe the effective amount of the drug required to prevent, counter or arrest the progress of the condition.

Dosages of the present invention, when used for the purpose of activating or stimulating immune cells, will generally range between about 0.01 mg per kg of body weight per day (mg/kg/day) to about 100 mg/kg/day, preferably 0.01 to 10 mg/kg/day, and most preferably 0.1 to 5.0 mg/kg/day. For intravenous administration, when used for the purpose of activating or stimulating immune cells, the compositions are preferably provided in dosage forms containing from about 0.01 milligrams to about 5 g, or up to about 10 g, of the API for the symptomatic adjustment of the dosage to the patient to be treated. A medicament typically contains from about 0.01 mg to about 500 mg of the API, preferably, from about 1 mg to about 200 mg of API. In one illustrative example, intravenously, a dose in a range from about 0.1 to about 100 mg/kg/minute can be administered during a constant rate infusion.

Preferred dosages and administration regimens of γδT cell activators phosphoantigen for the treatment of cancer for example are provided in co-pending PCT application number PCT/IB2003/006375 filed Dec. 2, 2003. Dosages and administration regimens for use as adjuvant in combination with an antigen for the treatment or prevention of disease, particularly infectious disease and cancer, are provided in co-pending PCT application number PCT/IB2005/001485 filed Apr. 25, 2005, PCT/IB2005/000509 filed Feb. 8, 2005 and U.S. Provisional patent application No. 60/637,619 field Dec. 20, 2004. Methods for treating disorders associated with airway hyperresponsiveness are provided in PCT/US00/26684 and U.S. Pat. No. 6,737,398. The disclosures of each of the foregoing references are incorporated herein by reference.

Advantageously, the crystalline phosphoantigens may be administered in a single daily dose, or the total daily dosage may be administered in divided doses of two, three or four times daily. Furthermore, the crystalline phosphoantigens can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The composition, shape, and type of dosage forms of the invention will typically vary depending on their use. For example, a dosage form used in the acute treatment of a disease or disorder may contain larger amounts of the active ingredient than a dosage form used in the chronic treatment of the same disease or disorder. Similarly, a parenteral dosage form may contain smaller amounts of the active ingredient than an oral dosage form used to treat the same disease or disorder.

In the methods of the present invention, the crystalline phosphoantigens can form the API, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

### Peak Plasma Concentrations

Hosts, including humans, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

Preferred dose of the compound for the purposes of activating or stimulating immune cells, particularly gamma delta T cells, are further described herein. The effective dosage range of the medicament composition to be delivered can be calculated based on the weight of the phosphoantigen compound to be delivered. If another element of the medicament exhibits activity in itself, the effective dosage can be estimated as above using the weight of the other element, or by other means known to those skilled in the art. Ideally a γδT cell activator phosphoantigen should be administered to achieve peak plasma concentrations of the active compound of from about 0.01 μM to 10 mM, preferably about 1 μM to 10 mM preferably about 10 μM to about 1 mM. This may be achieved, for example, by the intravenous injection of a 0.1 to 5% solution of the active ingredient, or as an oral dosage form.

The concentration of active compound in the drug composition will depend on absorption, inactivation and excretion rates of the drug as well as other factors known to those of skill in the art. It is to be noted that dosage values will also vary with the severity of the condition to be alleviated, or in the case of a γδT cell activator, the degree or nature of the activation or stimulation desired. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

### Oral Dosage Forms

Pharmaceutical compositions of the invention that are suitable for oral administration can be presented as discrete dosage forms, such as, but not limited to, tablets (including without limitation scored or coated tablets), pills, granules, lozenges, caplets, capsules, chewable tablets, powder packets, cachets, troches, wafers, aerosol sprays, or liquids, such as but not limited to syrups, elixirs, solutions or suspensions in an aqueous liquid, a non-aqueous liquid, an oil-in-water emulsion, or a water-in-oil emulsion. Such compositions contain a predetermined amount of a pharmaceutically acceptable phosphoantigen salt, and may be prepared by methods of pharmacy well known to those skilled in the art. See generally, Remington's Pharmaceutical Sciences, 18′ ed., Mack Publishing, Easton, Pa. (1990).

Typical oral dosage forms of the invention are prepared by combining the pharmaceutically acceptable phosphoantigen salt in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, (a) surface stabilizers, (b) dispersion aid, (c) binders, (d) filling agents, (e) lubricating agents, (f) glidants, (g) suspending agents, (h) sweeteners, (i) flavoring agents, (j) preservatives, (k) buffers, (1) wetting agents, (m) disintegrants, (n) effervescent agents, (o) humectants, (p) controlled release agents, (q) absorption accelerators, (r) absorbents, (s) plasticisers.

Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent.

In particular, examples of excipients that can be used in oral dosage forms of the invention include, but are not limited to, binders, fillers, disintegrants and lubricants.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Suitable forms of microcrystalline cellulose include, but are not limited to, the materials sold as AVICEL-PH-101, AVICEL-PH-103, Avicel PH 102, Avicel PH 112 and Avicel PH 302 AVICELRC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103 and Starch 1500 LM.

Examples of fillers suitable for use in the pharmaceutical compositions and dosage forms disclosed herein include, but are not limited to, talc, calcium carbonate (e.g., granules or powder), calcium phosphate, microcrystalline cellulose, powdered cellulose, lactose, dextrates, kaolin, mannitol, silicic acid, sorbitol, sucrose, maltodextrin, starch, pre-gelatinized starch, polymethacrylates, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may swell, crack, or disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the invention. The amount of disintegrant used varies based upon the type of formulation and mode of administration, and is readily discernible to those of ordinary skill in the art. Typical pharmaceutical compositions comprise from about 0.5 to about 15 weight percent of disintegrant, preferably from about 1 to about 5 weight percent of disintegrant. Disintegrants that can be used to form pharmaceutical compositions and dosage forms of the invention include, but are not limited to, agar-agar, alginic acid, guar gum, calcium carbonate, microcrystalline cellulose, croscarmellose sodium, carboxymethylcellulose calcium, methylcellulose, crospovidone, polacrilin potassium, sodium starch glycolate, potato or tapioca starch, other starches, pre-gelatinized starch, clays, other algins, other celluloses, gums, and mixtures thereof.

Lubricants that can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), sodium benzoate, sodium stearylfumarate, zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

This invention further encompasses lactose-free pharmaceutical compositions and dosage forms, wherein such compositions preferably contain little, if any, lactose or other mono- or di-saccharides. As used herein, the term “lactose-free” means that the amount of lactose present, if any, is insufficient to substantially increase the degradation rate of an active ingredient.

Lactose-free compositions of the invention can comprise excipients which are well known in the art and are listed in the USP (XXI)/NF (XVI), which is incorporated herein by reference. In general, lactose-free compositions comprise a pharmaceutically acceptable phosphoantigen salt (e.g., phosphoantigen sodium), a binder/filler, and optionally a lubricant, in pharmaceutically compatible and pharmaceutically acceptable amounts. Preferred lactose-free dosage forms comprise a pharmaceutically acceptable salt of phosphoantigen, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

### Controlled and Delayed Release Dosage Forms

Pharmaceutically acceptable phosphoantigen salts can be administered by controlled- or delayed-release means. Controlled-release pharmaceutical products have a common goal of improving drug therapy over that achieved by their non-controlled release counterparts.

Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Chemgju, Controlled Release Dosage Form Design, 2 (Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the phosphoantigen salts and compositions of the invention.

Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185; each of which is incorporated herein by reference. These dosage forms can be used to provide slow or controlled-release of one or more active ingredients using, for example, alginic acid, aliphatic polyesters, bentonite, cellulose acetate, phthalate, carnuba wax, chitosan, ethylcellulose, guar gum, microcrystalline wax, paraffin, polymethacrylates, povidone, xanthan gum, yellow wax, carbomers, hydroxypropylcellulose, hydroxypropylmethylcellulose, methylcellulose, other polymer matrices, gels, permeable membranes, osmotic systems (such as OROSX (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of phosphoantigen and thus effect controlled delivery of the drug. Examples of specific anion exchangers include, but are not limited to Duolite A568 and DuoliteAP143 (Rohm & Haas, Spring House, Pa. USA).

One embodiment of the invention encompasses a unit dosage form which comprises a pharmaceutically acceptable phosphoantigen salt (e.g., a sodium, potassium, or lithium salt), or a polymorph, solvate, hydrate, dehydrate, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS (Alza Corporation, Mountain View, Calif. USA). This technology can readily be adapted for the delivery of compounds and compositions of the invention. Various aspects of the technology are disclosed in U.S. Pat. Nos. 6,375,978; 6,368,626; 6,342,249; 6,333,050; 6,287,295; 6,283,953; 6,270,787; 6,245,357; and 6,132,420; each of which is incorporated herein by reference. Specific adaptations of OROS that can be used to administer compounds and compositions of the invention include, but are not limited to, the OROS Push-Pull, Delayed Push-Pull, Multi-Layer Push-Pull, and Push-Stick Systems, all of which are well known. See, e.g., http://www.alza.com. Additional OROS systems that can be used for the controlled oral delivery of compounds and compositions of the invention include OROS-CT and L-OROS; see also, Delivery Times, vol. II, issue II (Alza Corporation).

Conventional OROS oral dosage forms are made by compressing a drug powder (e.g., phosphoantigen salt) into a hard tablet, coating the tablet with cellulose derivatives to form a semi-permeable membrane, and then drilling an orifice in the coating (e.g., with a laser). Kim, Cherng, Controlled Release Dosage Form Design, 231-238 (Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS drug delivery systems cannot be used to effectively delivery drugs with low water solubility. Because phosphoantigen salts and complexes of this invention (e.g., phosphoantigen sodium) can be more soluble in water than e.g. amorphous phosphoantigen sodium salt or phosphoantigen starting material, they are well suited for osmotic-based delivery to patients.

Consequently, a specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent the exit orifice and in direct or indirect contacting relationship with the expandable layer; and a flow-promoting layer interposed between the inner surface of the wall and at least the external surface of the drug layer located within the cavity, wherein the drug layer comprises a salt of phosphoantigen, or a polymorph, solvate, hydrate, dehydrate, anhydrous, or amorphous form thereof. See U.S. Pat. No. 6,368,626, the entirety of which is incorporated herein by reference.

Another specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a salt of phosphoantigen, or a polymorph, solvate, hydrate, dehydrate, anhydrous, or amorphous form thereof. See U.S. Pat. No. 6,342,249, the entirety of which is incorporated herein by reference.

### Parenteral Dosage Forms

Parenteral dosage forms can be administered to patients by various routes, including, but not limited to, subcutaneous, intravenous, intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared.

Suitable vehicles that can be used to provide parenteral dosage forms of the invention are well known to those skilled in the art. Examples include, without limitation: sterile water; Water for Injection USP; saline solution; glucose solution; aqueous vehicles such as but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Compounds that alter or modify the solubility of a pharmaceutically acceptable salt of phosphoantigen disclosed herein can also be incorporated into the parenteral dosage forms of the invention, including conventional and controlled-release parenteral dosage forms.

### Topical, Transdermal and Mucosal Dosage Forms

Topical dosage forms of the invention include, but are not limited to, creams, lotions, ointments, gels, shampoos, sprays, aerosols, solutions, emulsions, and other forms know to one of skill in the art. See, e.g. Remington's Pharmaceutical Sciences, 18th ed., Mack Publishing, Easton, Pa. (1990); and Introduction to Pharmaceutical Dosage Forms, 4^{th} ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure.

Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18th Ed., Mack Publishing, Easton, Pa. (1990).

Transdermal and mucosal dosage forms of the invention include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences,18th Ed., Mack Publishing, Easton, Pa. (1990); and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer the active ingredient (s) of the invention include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466,465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference.

Suitable excipients (e.g., carriers and diluents) and other materials that can be used to provide transdermal and mucosal dosage forms encompassed by this invention are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with pharmaceutically acceptable salts of phosphoantigen of the invention. For example, penetration enhancers can be used to assist in delivering the active ingredients to or across the tissue. Suitable penetration enhances include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient (s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient (s) so as to improve delivery. In this regard, stearates can serve as a lipid vehicle for the formulation, as an emulsifying agent or surfactant, and as a delivery-enhancing or penetration-enhancing agent. Different hydrates, dehydrates, solvates, polymorphs, anhydrous, or amorphous forms of the pharmaceutically acceptable salt of phosphoantigen can be used to further adjust the properties of the resulting composition.

### Preferred Doses

The compounds of the invention can be used in the treatment of a range of disorders, depending on the compound and its particular therapeutic properties and indications. In one example the phosphoantigen compound leads to activation or expansion of γδT cells, and the disorder is an immune disorder, or a disorder where immunostimulation is desired, for example in cancer or infectious disease, or in vaccination. The compounds may also be useful in the treatment of autoimmune disorders, allergy or asthma and/or for treatment of airway hyperresponsiveness. Exemplary compounds include bisphosphonate compounds, BrHPP, EpoxPP, HDMAPP, C-HDMAPP, and IPP, the chemical structures for which are described herein.

The invention provides substantially or essentially pure compositions and formulations, preferably in dosage form for administration to a human. A preferred dosage form comprises a composition comprising substantially or essentially pure phosphoantigen, optionally together with a pharmaceutical carrier, the phosphoantigen present in a quantity of:
(1) compositions of Formula IIIa and IIIb and their derivatives (IIIa1, IIIa2, IIIa3, A, B, IIIb, IIIb1, IIIb2, IIIb3, C):
   a. between about 0.1 mg/kg and 100 mg/kg, or preferably between about 10 mg/kg and about 100 mg/kg, preferably between about 5 mg/kg and about 60 mg/kg, or about 10, 15, 20, 30, 40 or 50 mg/kg; or
   b. between about 5 mg and 10 g, or preferably between about 200 mg and about 10 g, preferably between about 200 g and about 1.2 g, or between about 200 mg/m² and 400, 600, 800, 1000, 1200, 1400, 1600 or 1800 mg/m² of body surface area.
(2) compositions of Formulas IIIc and its derivatives (IIIc, IIIc1, IIIc2, IIIc3, D, E, F and G):
   a. between about 1 μg/kg and about 100 mg/kg, or preferably between about 10 μg/kg and about 20 mg/kg, preferably between about 20 μg/kg and about 5 mg/kg, between about 20 μg/kg and 2.5 mg/kg, or about 0.1, 0.2, 0.3, 0.4 or 0.5 mg/kg; or
   b. between about 50 μg and 10 g, or preferably between about 100 μg and about 2 g, preferably between about 100 μg and about 0.5 g, or between about 5 mg/m² and 10, 50, 100, 200, 300, 500 or 1000 mg/m² of body surface area.

Preferably, the phosphoantigen is substantially or essentially non-hygroscopic. Preferably, the phosphoantigen has anionic purity that is at least 95%, 97%, 98%, 99%, 99.5%, 99.8% or 99.9%, as measured by HPAEC (expressed as relative area percent).

The method also provides for the large scale manufacturing of such compositions and thus includes batch quantities (for example at least 10 g, 20 g, 50 g, 100 g, 200 g, 300 g, 500 g, 750 g, 1 kg, 2 kg or greater) of substantially or essentially pure phosphoantigen compositions.

### Methods of Treatment

The phosphoantigens prepared according to the invention can be used in any suitable therapeutical protocol; it will be appreciated that different phosphoantigens will have different therapeutic utilities and will be used in the indications for which the active ingredient has been known or thought to be efficacious. For example, phosphoantigens may but are not required to have immunomodulatory (immunostimulatory or inhibitory) activity.

In one aspect the invention provides a vaccine including an immunostimulatory phosphoantigen molecule of the invention and an antigen. In another aspect the invention provides a method for inducing cytokine expression, for example interleukin expression, an INF (type I interferon; e.g. IFN-alpha, beta or gamma) expression, or TNF (tumor necrosis factor, e.g. TNF-alpha) expression. The method according to this aspect of the invention involves contacting a cell capable of expressing an interleukin, a type I IFN or a TNF with a phosphoantigen of the invention, in an effective amount to induce expression of said interleukin, type I IFN or TNF. In one aspect the invention provides a method for activating, either directly or indirectly, a γδT cell, a dendritic cell, a B cell, a T cell (e.g. CD8+ or CD4+), an NK.T cell, or a natural killer (NK) cell. The method according to this aspect of the invention involves contacting an immune cell, preferably a γδT cell, with a phosphoantigen of the invention, in an effective amount to activate said immune cell.

In one aspect the invention provides a method for treating an infection. The method according to this aspect of the invention involves administering a phosphoantigen of the invention to a subject having or at risk of developing an infection, in an effective amount to treat or prevent the infection. In one embodiment the subject has or is at risk of developing an infection chosen from a viral, bacterial, fungal or parasitic infection. In one aspect the invention provides a method for treating cancer. The method according to this aspect of the invention involves administering to a subject having or at risk of developing a cancer a phosphoantigen of the invention, in an effective amount to treat or prevent the cancer.

It will be appreciated that the invention also provides use of a phosphoantigen of the invention for manufacture of a medicament, particularly for any of the diseases or purposes mentioned herein, e.g. for use in treatment of a cancer, infection, vaccination, prevention, activating or stimulating immune cells, etc.

### BrHPP and EpoxPP in the Treatment of Cancer

The synthesis of BrHPP is described in Espinosa et al. (J. Biol. Chem., 2001, Vol. 276, Issue 21, 18337-18344), the disclosure of which is incorporated herein by reference. The synthesis of EpoxPP is described in European Patent No. 1109818B1, the disclosure of which is incorporated herein by reference.

(1) The present invention relates especially to the treatment of a disease, especially a tumor, especially a solid tumor, more especially one of the preferred diseases as defined above or below, characterized in that a compound of Formulas IIIa and IIIb and their derivatives (IIIa1, IIIa2, IIIa3, A, B, IIIb, IIIb1, IIIb2, IIIb3, C, especially BRHPP (A) or EpoxPP (C)), is administered more than once, with a two-weekly up to eight-weekly, preferably between three-weekly or four-weekly interval to a human in a dose that is calculated according to the formula (A):

single dose (mg/kg)=(0.1 to y)*N; (A)

where N (a whole or fractional number) is the number of weeks between treatments (about two to about eight weeks), that is N is about 2 to about 8, preferably between about 3 to 4; more preferably, the treatment dose is calculated according to the formula B,

single dose (mg/kg)=(5 to 100)*N; (B)

even more preferably according to the formula C,

single dose (mg/kg)=(10 to 100)*N; (C)

or still more preferably according to the formula D,

single dose (mg/m2)=(5 to 60)*N; (D)

where, in each of formulae A to D, N is about 2 to about 8 or preferably about 3 to 4 (corresponding to intervals of about 2 to about 8 weeks and about 3 to about 4 weeks between treatments); the compound of Formulas IIIa and 111b and their derivatives (IIIa1, IIIa2, IIIa3, A, B, IIIb, IIIb1, IIIb2, IIIb3, C, especially BRHPP (A), administration preferably taking place:

(a) about three-weekly to about four weekly, preferably three-weekly or four-weekly, in a human in a dose that lies between about 0.1 mg/kg and about 1.2 g/kg, preferably between about 10 mg/kg and about 1.2 g/kg, more preferably between about 5 mg/kg and about 100 mg/kg, even more preferably between about 5 mg/kg and 60 mg/kg, or preferably about 20 mg/kg; or

(b) about four-weekly to about eight weekly, preferably about five-weekly, six-weekly, seven-weekly or eight-weekly, in a human in a dose that lies dose is between about between about 0.1 mg/kg and about 1.2 g/kg, preferably between about 10 mg/kg and about 1.2 g/kg, more preferably between about 5 mg/kg and about 100 mg/kg, even more preferably between about 5 mg/kg and 60 mg/kg, or preferably about 20 mg/kg; the administration preferably taking place by i.v. infusion during 2 to 120 min, more preferably during about 5 to about 30 min, most preferably during about 10 to about 30 min, e.g. during about 30 min.

(2) The present invention preferably relates also to the treatment of a tumor disease, most preferably a tumor disease having metastases, said tumor being selected from a gastrointestinal, e.g. colorectal; lung tumor, especially a non-small cell lung carcinoma; a breast tumor; an epidermoid tumor; a renal; a genitourinary, e.g. prostatic; a pancreatic; and a brain tumor (and/or any metastasis thereof), most preferably a gastrointestinal tumor, especially a colorectal cancer, more especially a gastrointestinal cancer, especially a colorectal cancer; or a tumor of the genitourinary tract, especially a prostate cancer; where a compound of Formulas IIa and IIIb and their derivatives (IIIa1, IIIa2, IIIa3, A, B, IIIb, IIIb1, IIIb2, IIIb3, C) especially BRHPP (A) or EpoxPP (C), is administered to a warm-blooded animal, especially a human.

(3) The present invention also preferably relates to an in vivo regimen for stimulating a γδT cell in an individual, preferably a regimen for treatment of a tumor disease, preferably a solid tumor, or an autoimmune disorder or an infectious disease; wherein a composition is administered to an individual such that a compound of Formulas IIIa and IIIb and their derivatives (IIIa1, IIIa2, IIIa3, A, B, IIIb, IIIb1, IIIb2, IIIb3, C) especially BRHPP (A) or EpoxPP (C) is administered once in a dose that is

(a) between about the EC50 value and the EC100 value, more preferably at least 110%, 120%, 150% or 175% of the EC50, to a human

(b) between about 0.1 mg/kg and about 100 mg/kg, to a human

and, if required, one or more (preferably at least two, at least three, at least four, at least five, at least six, at least eight or at least ten) further doses each within the dose range mentioned above for the first dose are administered in further treatment cycles, preferably each dose after a period of time that allows for sufficient recovery of the γδT cell population to basal levels in the treated individual from each preceding dose administration, especially more than one week, more than two weeks after the preceding treatment, more especially two to eight weeks, most especially three to four weeks after the preceding treatment, especially three weeks after that treatment.

More preferably, under (1) to (3) a compound of Formulas IV to XIV, especially BRHPP or EpoxPP is administered three-weekly to a human in a dose that lies between about 0.1 mg/kg and about 1.2 g/kg, preferably between about 10 mg/kg and about 1.2 g/kg, more preferably between about 5 mg/kg and about 100 mg/kg, even more preferably between about 5 mg/kg and 60 mg/kg, or preferably about 20 mg/kg; or a compound of Formulas IV to XIV, especially BRHPP or EpoxPP is administered four-weekly (every 4 weeks) in a dose that is between about 0.1 mg/kg and about 1.2 g/kg, preferably between about 10 mg/kg and about 1.2 g/kg, more preferably between about 5 mg/kg and about 100 mg/kg, even more preferably between about 5 mg/kg and 60 mg/kg, or preferably about 20 mg/kg. This dose is preferably administered to the human by intravenous (i.v.) administration during 2 to 120 min, more preferably during about 5 to about 30 min, most preferably during about 10 to about 30 min, e.g. during about 30 min.

More preferably, said treatment is repeated until disease progression, unacceptable toxicity, 1 or preferably 2 cycles beyond determination of a complete response, or patient withdrawal of consent for any reason is encountered.

(4) The present invention preferably also relates to an in vivo regimen for the treatment of a tumor disease, especially (i) of a solid tumor selected from a gastrointestinal, e.g. colorectal; lung tumor, especially a non-small cell lung carcinoma; a breast tumor; an epidermoid tumor; a renal; a genitourinary, e.g. prostatic; a pancreatic; and a brain tumor (and/or any metastasis thereof), most preferably a gastrointestinal tumor, especially a colorectal cancer, more especially a gastrointestinal cancer, especially a colorectal cancer; or a tumor of the genitourinary tract, especially a prostate cancer; especially where such tumor is metastatic, wherein a compound of Formulas IIIa and IIIb and their derivatives (IIIa1, IIIa2, IIIa3, A, B, IIIb, IIIb1, IIIb2, IIIb3, C) especially BRHPP (A) or EpoxPP (C), is administered between once-weekly and eight-weekly to a warm-blooded animal in a dose that is below 80%, more preferably below 50% of the maximal tolerable dose (MTD) or highest dose tested in non-human animals.

### HDMAPP, CHDMAPP, NHDMAPP and H-angelylPP in the Treatment of Cancer

Methods for the synthesis of HDMAPP are described in Wolff et al, Tetrahedron Letters (2002) 43:2555 and Hecht et al, Tetrahedron Letters (2002) 43: 8929, the disclosures of which are incorporated herein by reference for their teaching of methods of preparing HDMAPP compounds. Methods for the synthesis of CHDMAPP is referred to in the section titled “Examples”. Structures and methods for the synthesis of NHDMAPP are provided in PCT patent application no. PCT/IB2004/004311 filed Dec. 2, 2004, the disclosure of which is incorporated herein by reference. Structures and methods for the synthesis of H-angelylPP are provided in PCT patent application no. PCT/IB2006/001206 filed Mar. 21, 2006 and example 11, the disclosure of which is incorporated herein by reference.

(1) The present invention relates especially to the treatment of a disease, especially a tumor, especially a solid tumor, more especially one of the preferred diseases as defined above or below, characterized in that a compound of Formulas IIIc and its derivatives (IIIc, IIIc1, IIIc2, IIIc3, D, E, F and G): especially HDMAPP (D) or C-HDMAPP (E), is administered more than once, with a two-weekly up to eight-weekly, preferably between three-weekly and four-weekly interval to a human in a dose that is calculated according to the formula (A)

single dose (mg/kg)=(0.1 to y)*N (A)

where N (a whole or fractional number) is the number of weeks between treatments (about two to about eight weeks), that is N is about 2 to about 8, preferably between about 3 to 4; more preferably, the treatment dose is calculated according to the formula B.

single dose (mg/kg)=(0.001 to 100)*N; (B)

even more preferably according to the formula C,

single dose (mg/kg)=(0.01 to 5)*N; (C)

or still more preferably according to the formula D,

single dose (mg/m²)=(0.02 to 2.5)*N (D)

where, in each of formulae A to D, N is about 2 to about 8 or preferably about 3 to 4 (corresponding to intervals of about 2 to about 8 weeks and about 3 to about 4 weeks between treatments); the compound of Formulas IIIc and its derivatives (IIIc, IIIc1, IIIc2, IIIc3, D, E, F and G), especially HDMAPP (D) or C-HDMAPP (E) administration preferably taking place:

(a) about three-weekly to about four weekly, preferably three-weekly or four-weekly, in a human in a dose that lies between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg, or preferably about 0.5 mg/kg;

(b) about four-weekly to about eight weekly, preferably about five-weekly, six-weekly, seven-weekly or eight-weekly, in a human in a dose that lies dose is between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg, or preferably about 0.5 mg/kg;

the administration preferably taking place by i.v. infusion during 2 to 120 min, more preferably during about 5 to about 30 min, most preferably during about 10 to about 30 min, e.g. during about 30 min.

(2) The present invention preferably relates also to the treatment of a tumor disease, most preferably a tumor disease having metastases, said tumor being selected from a gastrointestinal, e.g. colorectal; lung tumor, especially a non-small cell lung carcinoma; a breast tumor; an epidermoid tumor; a renal; a genitourinary, e.g. prostatic; a pancreatic; and a brain tumor (and/or any metastasis thereof), most preferably a gastrointestinal tumor, especially a colorectal cancer, more especially a gastrointestinal cancer, especially a colorectal cancer; or a tumor of the genitourinary tract, especially a prostate cancer; a said compound of Formulas IIIc and its derivatives (IIIc, IIIc1, IIIc2, IIIc3, D, E, F and G), especially HDMAPP (D) or C-HDMAPP (E), is administered to a warm-blooded animal, especially a human.

(3) The present invention also preferably relates to an in vivo regimen for stimulating a γδT cell in an individual, preferably a regimen for treatment of a tumor disease, preferably a solid tumor, or an autoimmune disorder or an infectious disease; wherein a compound of Formulas IIIc and its derivatives (IIIc, IIIc1, IIIc2, IIIc3, D, E, F and G), especially HDMAPP or C-HDMAPP is administered once in a dose that is

(a) between about the EC50 and the EC100, more preferably at least 110%, 120%, 150% or 175% of the EC50, to a human

(b) between about 10 μg/kg and about 20 mg/kg, to a human

and, if required, one or more (preferably at least two, at least three, at least four, at least five, at least six, at least eight or at least ten) further doses each within the dose range mentioned above for the first dose are administered in further treatment cycles, preferably each dose after a period of time that allows for sufficient recovery of the γδT cell population to basal levels in the treated individual from each preceding dose administration, especially more than one week, more than two weeks after the preceding treatment, more especially two to eight weeks, most especially three to four weeks after the preceding treatment, especially three weeks after that treatment.

More preferably, under (1) to (3) a compound of Formulas IIIc and its derivatives (IIIc, IIIc1, IIIc2, IIIc3, D, E, F and G), especially HDMAPP or C-HDMAPP is administered three-weekly to a human in a dose that lies between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg, or preferably about 0.5 mg/kg, or preferably about 0.5 mg/kg; or a compound of Formulas IIIc and its derivatives (IIIc, IIIc1, IIIc2, IIIc3, D, E, F and G), especially HDMAPP or C-HDMAPP is administered four-weekly (every 4 weeks) in a dose that is between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg, or preferably about 0.5 mg/kg. This dose is preferably administered to the human by intravenous (i.v.) administration during 2 to 120 min, more preferably during about 5 to about 30 min, most preferably during about 10 to about 30 min, e.g. during about 30 min.

More preferably, said treatment is repeated until disease progression, unacceptable toxicity, 1 or preferably 2 cycles beyond determination of a complete response, or patient withdrawal of consent for any reason is encountered.

(4) The present invention preferably also relates to an in vivo regimen for the treatment of a tumor disease, especially (i) of a solid tumor selected from a gastrointestinal, e.g. colorectal; lung tumor, especially a non-small cell lung carcinoma; a breast tumor; an epidermoid tumor; a renal; a genitourinary, e.g. prostatic; a pancreatic; and a brain tumor (and/or any metastasis thereof), most preferably a gastrointestinal tumor, especially a colorectal cancer, more especially a gastrointestinal cancer, especially a colorectal cancer; or a tumor of the genitourinary tract, especially a prostate cancer; especially where such tumor is metastatic, wherein a compound of Formulas IIIc and its derivatives (IIIc, IIIc1, IIIc2, IIIc3, D, E, F and G), especially HDMAPP or C-HDMAPP, is administered between once-weekly and eight-weekly to a warm-blooded animal in a dose that is below 80%, more preferably below 50% of the maximal tolerable dose (MTD).

Preferably, in the case of weekly treatment of a human with said a compound of Formulas XV to XXI, especially HDMAPP or C-HDMAPP, the dose is in the range of about 1 to about 60%, preferably about 10 to about 60%, e.g. about 5 to about 35% of the MTD, for example in the range of about 30 to about 35% of the MTD. Preferably, for HDMAPP and C-HDMAPP the dose is in the range of about 5 to about 60%, preferably about 10 to about 60%, especially in the range of about 10 to about 45%, most especially in the range of about 30 to about 45% of the MTD. In a special case, the dose can be between about 2 and about 18 mg/m² for HDMAPP and C-HDMAPP.

(5) The present invention preferably also relates to an in vivo regimen for the treatment of a disease, especially a solid tumor disease selected from a gastrointestinal, e.g. colorectal; lung tumor, especially a non-small cell lung carcinoma; a breast tumor; an epidermoid tumor; a renal; a genitourinary, e.g. prostatic; a pancreatic; and a brain tumor (and/or any metastasis thereof), most preferably a gastrointestinal tumor, especially a colorectal cancer, more especially a gastrointestinal cancer, especially a colorectal cancer; or a tumor of the genitourinary tract, especially a prostate cancer; especially where such tumor is metastatic, wherein a compound of Formulas IIIc and its derivatives (IIIc, IIIc1, IIIc2, IIIc3, D, E, F and G), especially HDMAPP (D), is administered between once-weekly and eight-weekly to a warm-blooded animal in a dose that is between the Efficient Concentration value giving half the maximum effect (EC50) and the Efficient Concentration value giving the maximal effect (EC100), or that is between 110% and 200% of the EC50, or preferably at least 110%, 120%, 130%, 150%, 160%, 175% or 200% of the EC50 value.

In further preferred aspects of any of the treatment methods described herein, the methods may comprise further administering a cytokine, preferably IL-2. For cancer, most preferably an interleukin-2 polypeptide, is administered over a period of time comprised between 1 and 10 days. Preferably, the interleukin-2 polypeptide is administered at a daily dose comprised between 0.2 and 10 MU (million units) per day, even more preferably between 0.2 and 1.5 MU, further preferably between 0.2 and 1 MU. The daily dose of cytokine, preferably an interleukin-2 polypeptide, is administered as a single injection or in two injections. Preferably the γ [symbol] T cell activator is administered as a single dose at the beginning of the treatment.

### Procedures

### Procedure for XRPD Analyses

### First Procedure:

X-Ray Powder Diffraction measurements were carried out by using a SIEMENS D5005 diffractometer (Bruker analytical X-Ray Systems, D-76187 Karlsruhe, Germany) with a Bragg Brentano geometry, in theta-theta reflection mode. The instrument is equipped with a X-Ray tube (copper anticathode, 40 kV, 30 mA, 1 radiation: 1.540598 Å, K_{α2} radiation: 1.544426 Å), a nickel filter and a scintillation detector. The diffraction patterns were collected by steps of 0.04° (2-theta) over the angular range 3°-30°, with a counting time of 4 s per step. No internal standard was used but a sample of quartz was analyzed as an external standard. The temperature of the sample was either the room temperature or accurately monitored when heating was required. DIFFRAC PLUS Edit Job software (v. 2.00) was used as control software and data processing was performed by using the Eva software (v. 9.0 and v. 10.0).

### Second Procedure.

X-Ray Powder Diffraction measurements were carried out by using a SIEMENS D5000 Matic diffractometer (Bruker analytical X-Ray Systems, D-76187 Karlsruhe, Germany) with a Bragg Brentano geometry, in theta-theta reflection mode. The instrument is equipped with a X-Ray tube (copper anticathode, 40 kV, 40 mA, K_{α1} radiation: 1.540598 Å, K_{α2} radiation: 1.544426 Å), a nickel filter and a scintillation detector. The diffraction patterns were collected by steps of 0.04° (2-theta) over the angular range 3°-30°, with a counting time of 4 s per step. No internal standard was used but a sample of quartz was analyzed as an external standard. The analyses were carried out at room temperature. DIFFRAC PLUS Edit Job software (v. 2.00) was used as control software and data processing was performed by using the Eva software (v. 9.0).

### Procedure for DSC Analyses

DSC analyses were carried out by using a SETARAM 141 Differential Scanning Calorimeter (SETARAM, 69300 Caluire, France). No purge gas was used. The reference material was an empty aluminum pan without cap. The samples were weighed in open aluminum pans, then placed in the analyzer. The analyses were performed within various temperature ranges using a 2°.min⁻¹ heating rate. SETARAM ACQUISITION Module software (v. 1.4) was used as control software and data processing was performed by using the SETARAM DATA PROCESSING Module software (v. 1.39).

### Procedure for TGA Analyses

TGA measurements were carried out by using a NETZSCH STA 409PC Thermogravimetric Analyzer (NETZSCH, D-95100 Selb, Germany). The purge gas used was dry nitrogen (gas flow=50 mL/min) and the reference material was an empty aluminum pan without cap. The samples were weighed in open aluminum pans, then placed in the thermogravimeter. The analyses were performed within various temperature ranges using a 2°.min⁻¹ heating rate. PROTEUS software was used for the acquisition and data processing.

### Procedure for TG/DSC Analyses

TG/DSC measurements were carried out by using a NETZSCH STA 449C Jupiter Thermogravimetric Analyzer (NETZSCH, D-95100 Selb, Germany) equipped with a low temperature furnace. The purge gas used was helium (gas flow=60 mL/min) and the reference material was an empty aluminum pan without cap. The samples were weighed in open aluminum pans, then placed in the analyzer. The analyses were performed within various temperature ranges using a 2°.min⁻¹ heating rate. PROTEUS software was used for the acquisition and data processing (v. 4.7.0).

### Procedure for Hygroscopicity and Moisture Measurements

### First Procedure:

Moisture sorption isotherms of amorphous BrHPP was obtained by using an automated water sorption analyzer. Temperature of analysis was 20° C. For each measurement, precisely ca. 25 mg of the sample were weighed in a pan beforehand allowed for the tare, then placed in the analyzer.

After a stabilization stage at 0% R.H. (R.H. stands for Relative Humidity), mass variations were recorded while R.H. was increased by steps of 20% R.H. The upper limit was adjusted to 80% R.H. This second stage was afterward followed by a stepped decrease of R.H. down to 0% R.H. This overall procedure constitutes a ‘Full cycle’ analysis. Several successive full cycles can be applied to the same sample.

### Second Procedure:

Moisture sorption isotherms of the crystalline phases were obtained by using a DVS-1 automated water sorption analyzer (Surface Measurements Systems, Alperton, Middlesex, United Kingdom). The reference material was an empty glass pan and the total gas flow was 200 sccm·min⁻¹ (Standard Cubic Centimeters per minute). Temperature of analysis was 22° C. For each measurement, precisely ca. 15 mg of the sample were weighed in a glass pan beforehand allowed for the tare, then placed in the analyzer.

As a first stage, dry nitrogen (0% R.H) was applied until constant weight of the sample in order to eliminate adsorbed or absorbed water (potential dehydration). Then, mass variations were recorded while R.H. was increased by steps of 10% R.H. The automated analyzer was allowed to start the following step as soon as the mass variation of the sample was less than 5.10⁻⁴ mg·min⁻¹. The upper limit was adjusted below the deliquescence of the compound and never exceeded 98% R.H. This second stage was afterward followed by a stepped decrease of R.H. down to 0% R.H. This overall procedure constitutes a ‘Full cycle’ analysis. Several successive full cycles can be applied to the same sample.

DVSWin (v 2.18) was used as control and data collection software whereas data processing was performed by using DVS Standard Analysis Suite (v 4.3).

Further parameters used in DVS procedures are as follows:
Protocole Dynamic Vapour Sorption/Appareil ‘Surface Measurements Systems
Automated Water Sorption Analyser’/model: DVS-1
Analysis method:
   step mode,
   Temperature: 25° C.
   number of steps,
   step dm/dt<0.0005 (mg/min)
   First stage R.H.=0%
   Last Stage R.H.=98%
   Half cycle or Full cycle (or several successive full cycles)
   dm/dt window: 5
   Minimum Stage: 20 min
   Maximum Stage: 120 min
   Total Gaz Flow: 200
   Nitrogen: 200 Bars, H2O<3 ppm, O2<2 ppm, Ref. Azote N50 B50*0
   Initial sample weight:
Software—data collection and data processing
   DVSWin v2.18 (control software),
   DVS Standard Analysis Suite v4.3 (analysis software).]

### Procedure for HPAEC Analysis

The chemical purity of phosphoesters (including the isomeric purity for geometric isomers) is determined using High Performance Anion-Exchange Chromatography (HPAEC) with suppressed conductivity detection. The HPAEC device comprises a DIONEX DX600 system (DIONEX corporation, Sunnyvale Calif., USA) connected to computer loaded with DIONEX Chromeleon® chromatography software. A DIONEX CD25 conductivity detector is used with an anion selfregenerating suppressor (ASRS®-ultra II—4 mm), which is set to either auto-suppression mode or external water mode. The HPAEC column used is a DIONEX Ion Pac® AS11 column (4×250 mm) equipped with an AG11 guard column (4×50 mm). In this procedure, phosphoesters are eluted (as anionic form) from the anion exchange column with a sodium or potassium hydroxide step gradient.

### EXAMPLES

### Phosphoantigen Compounds

TABLE 2
Phosphoantigen designation, chemical name and structure
(as acid form) cited in examples

| Designa- | | |
|---|---|---|
| tion | Chemical name | Structure (as acid form) |
| BrHPP | 3-(bromo-methyl)-3-butanol-1-ylpyrophosphoricacid | 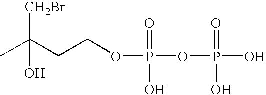 |
| IPP | 3-methyl-3-buten-1-ylpyrophoshoricacid | 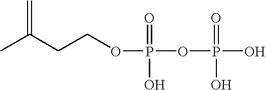 |
| C-HDMAPP | (2E)-1-Hydroxy-2-methyl-2-pentenyl-pyrophosphonicacid | 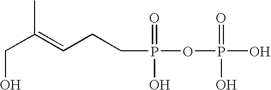 |

The synthesis of BrHPP is described in Espinosa et al. (J. Biol. Chem., 2001, Vol. 276, Issue 21, 18337-18344) or U.S. Pat. No. 6,660,723, the disclosure of which is incorporated herein by reference. The synthesis of EpoxPP is described in European Patent No. 1109818B1.

The synthesis of CHDMAPP can be carried according to any suitable method. Examples include the methods of Nakamura et al, Tetrahedron Letters no 2, p 111-112 (1973), Zoretic and Zhang, Tetrahedron Letters, vol 37-11, p 1751-1754 (1996) or Umbreit and Sharpless, JACS, 99-16, pp 5526-5527 (1977), the disclosures of which are incorporated herein by reference, to produce a E-hydroxydimethylallyl type synthon prior to phosphorylation or phosphonation. Phosphorylation or phosphonation can then be carried out according to methods described in PCT patent publication no. WO 03/050128, Brondino et al, Journal of Fluorine Chemistry, 76, p 193-200 (1996), or Valentijn et al., Syn. Lett., p 663-664 (1991), the disclosures of which are incorporated herein by reference. The preparation of IPP is described in U.S. Pat. No. 5,639,653.

### Example 1

### Preparation of 3-(bromomethyl)-3-butanol-1-yl diphosphoric acid, diquininium salts (BrHPP Quinine Salts)

Preparation of BrHPP Quinine Salts Crystalline (n,p)-Mix-I:

25.45 g (49.7 mmoles, 1 equivalent) of a racemic mixture of BrHPP sodium salt (INNATE PHARMA, Marseilles, France—batch INP-D004-03a, 86% purity based on HPAEC analysis) is dissolved in 100 mL of deionized water at room temperature. The aqueous solution obtained is loaded on a column containing 1.216 equivalents (553 mL) of IMAC 1100 cationic resin (H⁺ form), then eluted with 650 mL of deionized water. After elution, the volume of the resulting acidic solution of BrHPP is ca. 750 mL. An equal volume of ethanol is added to this solution. An ethanolic solution of the quinine base which was prepared beforehand by dissolving 32.26 g of anhydrous quinine (99.5 mmoles, 2 equivalents, 99% purity w/w, purchased from ACROS®, Belgium) in 300 mL of ethanol is then added slowly while stirring to the acidic solution of BrHPP (salification step). The entire amount of solvent is removed by evaporation under reduced pressure at 40° C. and successive stages of distillation are performed with addition of 200 mL portions of ethanol in order to obtain a dry residue. Approx. 56.6 g of yellowish foam is obtained at this stage. In order to perform the crystallization, the dry residue is dissolved in a minimum amount of ethanol (ca. 250 mL) and deionized water is slowly added while stirring at ambient temperature. The addition of water is stopped when a high turbidity of the slurry is obtained (approx. 1 L of water). The solid phase is then collected by filtration through a glass filter and allowed to dry at ambient atmosphere until constant weight. 40.5 g of the crystalline (n,p)-Mix-I of BrHPP Quinine salts are obtained by this procedure with a yield of approx. 78%.

Preparation of BrHPP Quinine Salt, Crystalline (n,p)-Mix-II:

Starting from 30° C. (n,p)-Mix-I is progressively heated by 10° C. steps (45 minutes between each increment). At 70° C., (n,p)-Mix-II is obtained.

Preparation of BrHPP Quinine Salt, Crystalline (n,p)-Mix-III:

Starting from 30° C. (n,p)-Mix-I is progressively heated by 10° C. increments (45 minutes between each increment). At 100° C., (n,p)-Mix-III is obtained.

### X-Ray Powder Diffraction (XRPD) Analyses:

The XRPD patterns of (n,p)-Mix-I, (n,p)-Mix-II and (n,p)-Mix-III are shown respectively in FIG. 1, FIG. 2 and FIG. 3 (the background has not been removed). For each XRPD pattern, diffraction angles (2- [symbol] ), intensity and relative intensity (expressed as a percentage of the most intense ray) of the significant peaks are collected in the associated tables. The analysis of (n,p)-Mix-I was carried out according to the second procedure (SIEMENS D5000 Matic), whereas the two others were carried out according to the first procedure (SIEMENS D5005, at 70° C. for (n,p)-Mix-II and 100° C. for (n,p)-Mix-III).

Composition of BrHPP Quinine Salts, Crystalline (n,p)-Mix-I:

The stoichiometry of the crystalline BrHPP Quinine salts (n,p)-Mix-I stored under ambient conditions is 1 molecule of BrHPP, 2 molecules of Quinine and 3 molecules of water. In this example, the quantity of water is determined by the mean of thermogravimetric analysis as shown in FIG. 1′ using the first procedure (NETZSCH STA 409PC Thermogravimetric Analyzer).

The average mass loss after several analysis is 5.15% which corresponds to 3 molar equivalents of water (theoretical mass variation: Δmₜₕ=5.16%). The dehydration occurs between 55° C. and 100° C. If the anhydrous compound which corresponds to BrHPP crystalline (n,p)-Mix-III is stored under 100% Relative Humidity (RH) at ambient temperature (ca. 20° C.) for 24 hours, the tri-hydrated crystalline BrHPP Quinine (n,p)-Mix-I is recovered: the phenomenon is thus reversible.

### Example 2

### Preparation of 3-(bromomethyl)-3-butanol-1-yl diphosphoric acid, di-cinchonidinium salts (BrHPP Cinchonidine Salts)

### Preparation of BrHPP Cinchonidine Salts in the Amorphous State:

1.120 g (2.187 mmoles, 1 equivalent) of racemic BrHPP sodium salt (INNATE PHARMA, Marseilles, France—batch INP-D004-03a, 86% purity based on HPAEC analysis) is dissolved in 5 mL of deionized water at room temperature. The aqueous solution obtained is loaded on a column containing 194 milliequivalents (88 mL) of IMAC 1100 cationic resin (H⁺ form), then eluted with 90 mL of deionized water. After elution, the volume of the resulting acidic solution of BrHPP is ca. 95 mL. 50 mL of ethanol is added to this solution. An ethanolic solution of the cinchonidine base which was prepared beforehand by dissolving 1.284 g of cinchonidine (4.362 mmoles, 2 equivalents, 98.5% minimum purity w/w, purchased from ACROS®, Belgium) in 40 mL of ethanol is then added slowly while stirring to the acidic solution of BrHPP (salification step). The resulting mixture is a homogeneous colourless solution. The entire amount of solvent is removed by evaporation under reduced pressure at 40° C. and successive stages of distillation are performed with addition of 50 mL portions of ethanol in order to obtain a dry residue. 2.266 g of amorphous BrHPP cinchonidine salts (yellowish foam) are obtained by this procedure.

Preparation of BrHPP Cinchonidine Salts Crystalline (n,p)-Mix-I:

462 mg of BrHPP cinchonidine salts in the amorphous state are homogenised in 23.3 g of deionized water while stirring at ambient temperature (≈20° C.). After a few minutes, a solid phase spontaneously nucleates that progressively leads to a white suspension. The entire amount of the solid is dissolved by increasing temperature at 70° C. (using a heating plate). Heating is then stopped and the system is kept on the plate allowing a slow cooling of the mixture down to ambient temperature (for several hours). By this way, a slow recrystallization is obtained. The solid phase is collected by filtration through a glass filter and washed with acetone, then allowed to dry at ambient atmosphere until constant weight. 25 mg of the white crystalline (n,p)-Mix-I of BrHPP cinchonidine salts are obtained by this procedure with a yield of ≈5%.

Preparation of BrHPP Cinchonidine Salts Crystalline (n,p)-Mix-II:

1.697 g of BrHPP cinchonidine salts in the amorphous state are homogenised in 5.93 g of a water/ethanol (75/25, wt. %) mixture while stirring at ambient temperature (≈20° C.). After a few minutes, a solid phase spontaneously nucleates that progressively leads to a white slurry. The system is kept at ambient temperature while stirring for 24 hours. The solid phase is collected by filtration through a glass filter and then allowed to dry at ambient atmosphere until constant weight. (n,p)-Mix-II of BrHPP cinchonidine salts is obtained by this procedure.

### X-Ray Powder Diffraction Analyses:

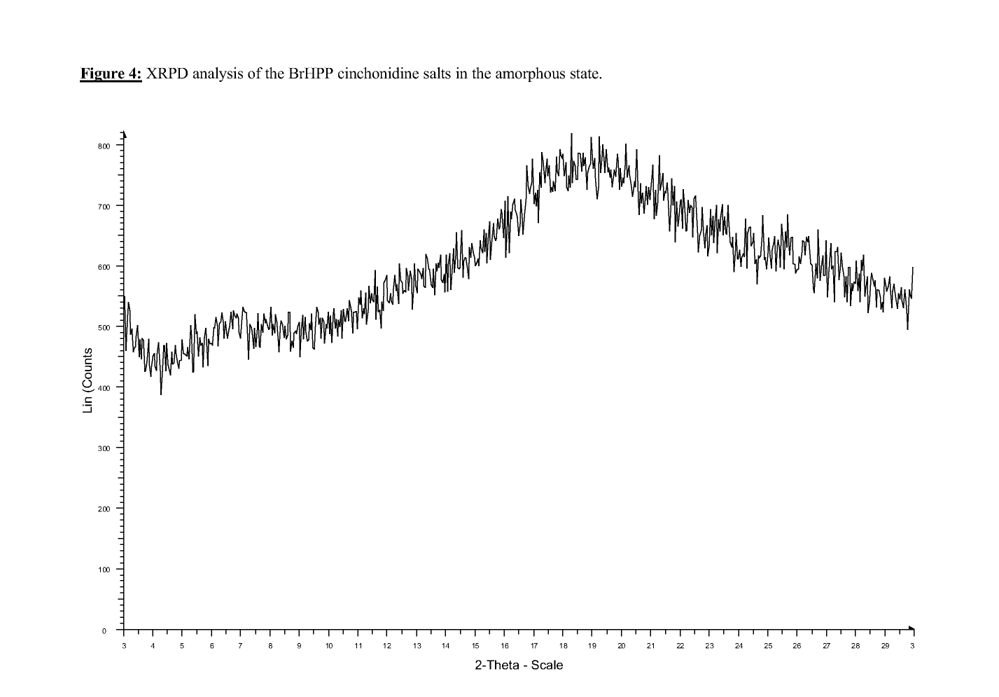

The XRPD patterns of the amorphous, (n,p)-Mix-I and (n,p)-Mix-II are shown respectively in FIG. 4, FIG. 5 and FIG. 6 (the background has not been removed). For (n,p)-Mix-I and (n,p)-Mix-II, diffraction angles (2-theta ) intensity and relative intensity (expressed as a percentage of the most intense ray) of the significant peaks are collected in the associated table. These analyses were carried out according to the first procedure (SIEMENS D5005, room temperature).

### Example 3

### Preparation of 3-(bromomethyl)-3-butanol-1-yl diphosphoric acid, di-8-hydroxyquinolinium salts (BrHPP 8-Hydroxyquinoline Salts)

### Preparation of BrHPP 8-Hydroxyquinoline Salts, Crystalline Phase-I:

1.034 g (2.019 mmoles, 1 equivalent) of racemic BrHPP sodium salt (INNATE PHARMA, Marseilles, France—batch INP-D004-03a, 86% purity based on HPAEC analysis) is dissolved in 5 mL of deionized water at room temperature. The aqueous solution obtained is loaded on a column containing 194 milliequivalents (88 mL) of IMAC 1100 cationic resin (H⁺ form), then eluted with 90 mL of deionized water. After elution, the volume of the resulting acidic solution of BrHPP is ca. 95 mL. 20 mL of methanol is added to this solution. A methanolic solution of the 8-hydroxyquinoline base which was prepared beforehand by dissolving 594 mg of 8-hydroxyquinoline (4.092 mmoles, 2 equivalents, beforehand purified by a recrystallization in diethyl ether) in 20 mL of methanol is then added slowly while stirring to the acidic solution of BrHPP (salification step). The resulting mixture is a homogeneous yellow solution. The entire amount of solvent is removed by evaporation under reduced pressure at 40° C. and successive stages of distillation are performed with addition of 50 mL portions of methanol in order to obtain a dry residue. 1.537 g of a yellow solid is obtained at this stage. This solid is dissolved at 60° C. in 20 mL of methanol (homogenous yellow solution). A cooling ramp (from 60° C. to 20° C. for 2 hours) is applied in order to induce the crystallization (seeding with a small amount of Phase-I at 28° C.). This crystallization is followed by a maturation step (24 temperature cycles between 30° C. and 10° C. with a 10°/h cooling rate). The last cycle is stopped at 18° C. (during the cooling phase) and the solid phase is collected by filtration through a glass filter and allowed to dry at ambient atmosphere until constant weight. 411 mg of the yellow crystalline Phase-I of BrHPP 8-hydroxyquinoline salt are obtained by this procedure with a yield of ≈32%.

### X-Ray Powder Diffraction Analysis:

The XRPD pattern of the crystalline Phase-I is shown in FIG. 7 (the background has not been removed). Diffraction angles (2- [symbol] ) intensity and relative intensity (expressed as a percentage of the most intense ray) of the significant peaks are collected in the associated table. This analysis was carried out according to the second procedure (SIEMENS D5000 Matic).

### Example 4

### Preparation of 3-(bromomethyl)-3-butanol-1-yl diphosphoric acid, N,N′-dibenzylethylenediammonium salts (BrHPP Benzathin Salts)

### Preparation of BrHPP Benzathin Salts Crystalline (Rac)-Mix-I:

1.080 g (2.109 mmoles, 1 equivalent) of racemic BrHPP sodium salt (INNATE PHARMA, Marseilles, France—batch INP-D004-03a, 86% purity based on HPAEC analysis) is dissolved in 5 mL of deionized water at room temperature. The aqueous solution obtained is loaded on a column containing 194 milliequivalents (88 mL) of IMAC 1100 cationic resin (H⁺ form), then eluted with 110 mL of deionized water. After elution, the volume of the resulting acidic solution of BrHPP is ca. 115 mL. An ethanolic solution of the benzathin base which was prepared beforehand by diluting 520 mg of benzathin (2.164 mmoles, 1 equivalent, 97% purity, purchased from SIGMA-ALDRICH®, Germany) in 20 mL of ethanol is then added slowly while stirring to the acidic solution of BrHPP (salification step). The resulting mixture is a colourless solution. The entire amount of solvent is removed by evaporation under reduced pressure at 40° C. and the drying is completed under dry nitrogen atmosphere for several hours. 1.383 g of a white solid is obtained at this stage. At 40° C., 78 g of deionized water are added to the dry residue while stirring, which leads to a white suspension of low viscosity. A slow cooling ramp (from 40° C. down to 2° C., t>4 hours) doesn't allow to significantly increase the quantity of crystalline material. 5.1 g of ethanol are added to this suspension (final solvent composition: 94/6, wt. %) and the system is fast cooled down to −7° C. (crystallization of the solvent-solid system). Then, the temperature is increased up to 0° C. and the slurry is kept under isothermal stirring for 16 hours. The solid phase is collected by filtration through a glass filter and washed with 20 mL of acetone, then allowed to dry at ambient atmosphere until constant weight. 521 mg of the racemic white crystalline (Rac)-Mix-I of BrHPP benzathin salts are obtained by this procedure with a yield of =51%.

### Preparation of BrHPP Benzathin Salts Crystalline (Rac)-Mix-II (Spontaneous Nucleation):

254 mg of BrHPP benzathin salt (Rac)-Mix-I (0.435 mmoles) are dissolved at 33° C. in 2.374 g of a acetic acid (50%, wt. %)-water (50%) mixture (acetic acid 100%, purchased from Prolabo, VWR International France). 7.974 g of methanol are added while stirring. Then, the spontaneous nucleation of a white solid phase occurs and progressively leads to a suspension of moderate viscosity. The solid phase is collected by filtration through a glass filter and allowed to dry at ambient atmosphere until constant weight. 0.182 g of the asolvated racemic white crystalline (Rac)-Mix-II of BrHPP benzathin salts are obtained by this procedure with a yield of 72%.

### Preparation of BrHPP Benzathin Salts Crystalline (Rac)-Mix-II (Seeded Crystallization):

259 mg of BrHPP benzathin salt (Rac)-Mix-I (0.444 mmoles) are dissolved at room temperature in 932 mg of a acetic acid (50%, wt. %)-water (50%) mixture (acetic acid 100%, purchased from Prolabo, VWR International France). 1.064 g of methanol are added while stirring. The clear solution is then seeded by a few milligrams of BrHPP benzathin salts crystalline (Rac)-Mix-II: the crystallization velocity appears to be relatively high. The resulting suspension is kept while stirring for one night. 5.289 g more methanol are added, that allows to decrease the viscosity of the suspension and thus facilitates the filtration. The solid phase is collected by filtration through a glass filter and allowed to dry at ambient atmosphere until constant weight. 0.193 g of the asolvated racemic white crystalline (Rac)-Mix-II of BrHPP benzathin salts are obtained by this procedure with a yield of 75%.

### Preparation of BrHPP Benzathin Salts Crystalline (Rac)-Mix-III:

249 mg of BrHPP benzathin salt (Rac)-Mix-II (0.427 mmoles) are slurried in 837 mg of water at room temperature. The system is kept for one night while stirring: (Rac)-Mix-III is obtained as a white paste (efflorescent dihydrate).

### X-Ray Powder Diffraction Analyses:

The XRPD patterns of the crystalline (Rac)-Mix-I, (Rac)-Mix-II and (Rac)-Mix-III are given in FIG. 8, FIG. 9 and FIG. 10 respectively (the background has not been removed). Diffraction angles (2- [symbol] ), intensity and relative intensity (expressed as a percentage of the most intense peak) of the significant peaks are collected in the associated tables. These analyses were carried out according to the second procedure (SIEMENS D5000 Matic).

### Example 5

### Preparation of (2E)-1-Hydroxy-2-methyl-2-pentenyl-pyrophosphonic acid, diquininium salts ((E)-C-HDMAPP Quinine Salts)

### Preparation of (E)-C-HDMAPP Quinine Salts Crystalline (E)-Phase-I:

861 mg (2.127 mmoles, 1 equivalent) of (E)-C-HDMAPP ammonium salt (batch NE-014271-A-3-2 crude 4#1, 80% purity based on HPAEC analysis) is dissolved in 10 mL of deionized water at room temperature. The aqueous solution obtained is loaded on a column containing 166 milliequivalents (83 mL) of DOWEX 50WX8-100 cationic resin (H⁺ form), then eluted with 140 mL of deionized water. After elution, the volume of the resulting acidic solution of (E)-C-HDMAPP is ca. 150 μL. 80 mL of ethanol are added to this solution. 16.5 mL of a 0.278 M ethanolic solution of the Quinine base (4.587 mmoles, 2.2 equivalents, 99% purity w/w, purchased from ACROS®, Belgium) is then added slowly while stirring to the acidic solution of (E)-C-HDMAPP (salification step). The resulting mixture is a colourless solution. The entire amount of the solvent is removed by evaporation under reduced pressure at 40° C. and successive stages of distillation are performed with addition of 50 mL portions of ethanol in order to obtain a dry residue. 2.148 g of (E)-C-HDMAPP quinine salts (yellowish solid) are obtained at this stage. At 45° C., 17.1 g of a water/methanol (58/42, wt %) mixture are added to the dry residue while stirring. It leads to a white suspension of low viscosity. The temperature is slowly decreased down to 20° C. to obtain a white slurry. The solid phase is collected by filtration through a glass filter and allowed to dry at ambient atmosphere until constant weight. 1.015 g of the white crystalline (E)-Phase-I of (E)-C-HDMAPP quinine salt are obtained by this procedure with a yield of ≈53%.

### Preparation of (E)-C-HDMAPP Quinine Salts, Crystalline (E)-Phase-II:

(E)-Phase-I is heated at 50° C. for 2 h 15 min. The resulting solid corresponds to a new crystalline phase of (E)-C-HDMAPP quinine salt referred to as (E)-Phase-II.

### X-Ray Powder Diffraction Analyses:

The XRPD patterns of (E)-Phase-I and (E)-Phase-II are shown respectively in FIG. 11 and FIG. 12 (the background has not been removed). Diffraction angles (2-thet [symbol] ), intensity and relative intensity (expressed as a percentage of the most intense ray) of the significant peaks are collected in the associated tables. These analyses were carried out according to the first procedure (SIEMENS D5005, room temperature).

### Example 6

### Preparation of (2E)-1-Hydroxy-2-methyl-2-pentenyl-pyrophosphonic acid, N,N′-dibenzylethylenediammonium salts ((E)-C-HDMAPP Benzathin Salts)

### Preparation of (E)-C-H DMAPP Benzathin Salt Crystalline (E)-Phase-I:

11.141 g of a 1.951% (wt. %) aqueous solution (0.699 mmoles, 1 equivalent) of (E)-C-HDMAPP triammonium salt (batch NE-014271-A-3-4 Chrom 1#3, 89% purity based on HPAEC analysis) is loaded on a column containing 194 milliequivalents (88 mL) of IMAC 1100 cationic resin (H⁺ form), then eluted with 90 mL of deionized water. After elution, the volume of the resulting acidic solution of (E)-C-HDMAPP is ca. 100 mL. An ethanolic solution of the benzathin base which was prepared beforehand by diluting 178.5 mg of benzathin (0.743 mmoles, 1.1 equivalent, 97% purity, purchased from SIGMA-ALDRICH®, Germany) in 20 mL of ethanol is then added slowly while stirring to the acidic solution of (E)-C-HDMAPP (salification step). The resulting mixture is a colourless solution. The entire amount of solvent is removed by evaporation under reduced pressure at 40° C. and successive stages of distillation are performed with addition of 50 mL portions of ethanol and acetone in order to obtain a dry residue. That allows to obtain 476 mg of (E)-C-HDMAPP benzathin salt at the amorphous state (mixture of white foam and colourless oil). A small amount of ethanol (<20 mL) is added while stirring, that leads to a white slurry. This mixture is kept under isothermal stirring for 2 hours, then filtered on a glass filter. The crop is allowed to dry under ambient atmosphere until constant weight. 337 mg of the crystalline (E)-Phase-I of (E)-C-HDMAPP benzathin salt are obtained by this procedure with a yield of ≈71%.

### Preparation of (E)-C-HDMAPP Benzathin Salt Crystalline (E)-Phase-II:

1.398 g of a 77% (wt. %) oil (1.500 mmoles, 1 equivalent) of (2E)-1-(2-Tetrahydropyranyloxy)-2-methyl-2-pentenyl-pyrophosphonate Bis-tributylammonium salt (THP protected C-HDMAPP, batch NE-014271-A-3-5 crude 14#1, 77% purity based on HPAEC analysis) is diluted with 5 mL of deionized water at room temperature. The aqueous solution obtained is loaded on a column containing 175 milliequivalents (88 mL) of DOWEX 50WX8-100 cationic resin (H⁺ form), then eluted with 145 mL of deionized water (deprotection/acidification step—see note below). After elution, the volume of the resulting acidic solution of (E)-C-HDMAPP is ca. 150 mL. 17.0 mL of a 0.100 M ethanolic solution of the benzathin base (1.700 mmoles, 1.1 equivalent, 97% purity, purchased from SIGMA-ALDRICH®, Germany) is then added slowly while stirring to the acidic solution of (E)-C-HDMAPP (salification step). The resulting mixture is a colourless solution. The entire amount of solvent is removed by evaporation under reduced pressure at 40° C. that allows to obtain 0.926 g of (E)-C-HDMAPP benzathin salt at the amorphous state (mixture of yellowish foam and oil). This residue is homogenized with 10.2 g of water while stirring at room temperature. The resulting solution is seeded with a small amount of the crystalline (E)-Phase-I. That leads to a brown slurry of high viscosity. The temperature is then progressively decreased down to 2° C. The solid phase is collected by filtration through a glass filter and allowed to dry at ambient atmosphere until constant weight. 192 mg of the crystalline (E)-Phase-II of (E)-C-HDMAPP benzathin salt are obtained by this procedure with a yield of =26%.

Note: a “one pot” deprotection/acidification sequence is conducted in this example since the THP protective group of the allylic alcohol can be easily removed under acidic conditions.

### X-Ray Powder Diffraction Analyses:

The XRPD patterns of (E)-Phase-I and (E)-Phase-II are shown respectively in FIG. 13 and FIG. 14 (the background has not been removed). Diffraction angles (2-thet [symbol] ), intensity and relative intensity (expressed as a percentage of the most intense ray) of the significant peaks are collected in the associated tables. These analyses were carried out according to the second procedure (SIEMENS D5000 Matic).

### Example 7

### Preparation of 3-methyl-3-buten-1-yl pyrophoshoric acid, diquininium salts (IPP Quinine Salts)

### Preparation of IPP Quinine Salt Crystalline Phase-I:

791 mg (2.535 mmoles, 1 equivalent) of IPP sodium salt (batch ChP4, 70% purity based on HPAEC analysis) is dissolved in 4 mL of deionized water at room temperature. This aqueous solution is loaded on a column containing 194 milliequivalents (88 mL) of IMAC 1100 cationic resin (H⁺ form), then eluted with 200 mL of deionized water. After elution, the volume of the resulting acidic solution of IPP is ca. 200 mL. 100 mL of ethanol are added to this solution. An ethanolic solution of the quinine base which was prepared beforehand by dissolving 1.644 g of quinine (5.068 mmoles, 2 equivalents, 99% purity w/w, purchased from ACROS®, Belgium) in 20 mL of ethanol is then added slowly while stirring to the acidic solution of IPP (salification step).The entire amount of solvent is removed by evaporation under reduced pressure at 40° C. and one complementary step of distillation is performed by using ethanol. 3.020 g of IPP quinine salt (colourless paste—amorphous state) are obtained at this stage. In order to perform the crystallization, this residue is homogenized in 20 mL of ethanol and water is slowly added while stirring (V_{H2O}=190 mL—ambient temperature), that leads to a white suspension of low turbidity. The mixture is then concentrated under reduced pressure at 40° C. The resulting suspension (V≈100 mL) is kept without stirring overnight (16 hours) at ambient atmosphere. This allows to obtain a white slurry. The solid phase is collected by filtration through a glass filter and allowed to dry at ambient atmosphere until constant weight. 1.142 g of the white crystalline Phase-I of IPP quinine salt are obtained by this procedure with a yield of ≈50%.

### Preparation of IPP Quinine Salt, Crystalline Phase-II:

A slurry is prepared with 858 mg of Phase-I of IPP quinine salt and 10 mL of acetone. This mixture is kept at 20° C. while stirring for 4 days. The solid phase is collected by filtration through a glass filter and allowed to dry at ambient atmosphere. Phase-II of IPP quinine salt is obtained by this procedure.

### X-Ray Powder Diffraction Analyses:

The XRPD patterns of Phase-I and Phase-II are shown respectively in FIG. 15 and FIG. 16 (the background has not been removed). Diffraction angles (2- [symbol] ) intensity and relative intensity (expressed as a percentage of the most intense ray) of the significant peaks are collected in the associated tables. These analyses were carried out according to the first procedure (SIEMENS D5005, room temperature).

### Example 8

### Chemical Purity and Stereoselectivity

The chemical purity of phosphoesters (including the isomeric purity for geometric isomers) is determined using High Performance Anion-Exchange Chromatography (HPAEC) with suppressed conductivity detection. As shown in table 3, the crystallization method according to the invention allows to increase the chemical purity (also referred to as the anionic purity) of phosphoantigen compounds. Table 3 shows the improvement in chemical purity after a single crystallization step; the single step may use for example as starting materials a crude or non-crystallized phosphoantigen, or a crystallized phosphoantigen, the latter starting materials generally being of higher chemical purity that the former and which step can also be considered a re-crystallization (see notes (b) and (d) below). In the case where geometric isomers are present (entries 7, 8, 9 and 10 of Table 3), the method according to the invention also leads to a stereoselective purification. An example of HPAEC profile is provided in FIG. 17.

TABLE 3
Chemical purities based on HPAEC analyses of phosphoantigen
compounds before and after a single crystallization step.

| | | Crystal- | Purity of | Purity of |
|---|---|---|---|---|
| Entry | Purified compound | lization | starting | purified |
| # | designation | solvent | material | compound |
| 1 | BrHPP Quinine salts, | MeOH/H₂O | <50%^{a} | 97.5% |
| 2 | crystalline (n,p)-Mix-I | MeOH/H₂O | 87.1%^{b} | 96.4% |
| 3 | | MeOH/H₂O | 94.7% | 99.2% |
| 4 | BrHPP Benzathin salts | MeOH/H₂O | <50%^{a} | 95.3% |
| | crystalline (Rac)-Mix-I | | | |
| 5 | BrHPP Benzathin salts | H₂0/ | 98.9%^{d} | 99.4% |
| | crystalline (Rac)-Mix-II | Ethylene | | |
| | | glycol | | |
| 6 | IPP quinine salt, | EtOH/H₂O | 95% | 99.3% |
| | crystalline Phase-III | | | |
| 7 | C-HDMAPP Quinine salt | MeOH/H₂O | 80% | 95.4% |
| | crystalline (E)-Phase-I | | (E/Z ratio: | (Z isomer |
| | | | 99.5/0.5) | not detected) |
| 8 | | MeOH/H₂O | 77%^{c} | 94.9% |
| | | | (E/Z ratio: | (Z isomer |
| | | | 98/2) | <0.5%) |
| 9 | | MeOH/H₂O | 87% (E) | 97.1% (E) |
| | | | (E/Z ratio: | (E/Z ratio: |
| | | | 94/6) | 98/2) |
| 10 | C-HDMAPP benzathin | H₂O | 77%^{c} | 94.4% |
| | salt, crystalline | | (E/Z ratio: | (Z isomer |
| | (E)-Phase-II | | 98/2) | <0.5%) |

^{a}The starting material is a crude product (bromation step crude product) containing one molar equivalent of free bromide.
^{b}The starting material is BrHPP Quinine salts, crystalline (n,p)-Mix-I.
^{c}The starting material corresponds to the crude THP protected C-HDMAPP as reported in Example 6.
^{d}The starting material is BrHPP Benzathin salts, crystalline (Rac)-Mix-I

### Example 9

### Chemical Stability Assessment

The chemical stability of crystalline forms according to the invention can be evaluated using DSC analyses and Ion Chromatography (HPAEC). Any other suitable method can be used as well, including by way of example phosphorous and proton NMR, reverse phase HPLC analysis.

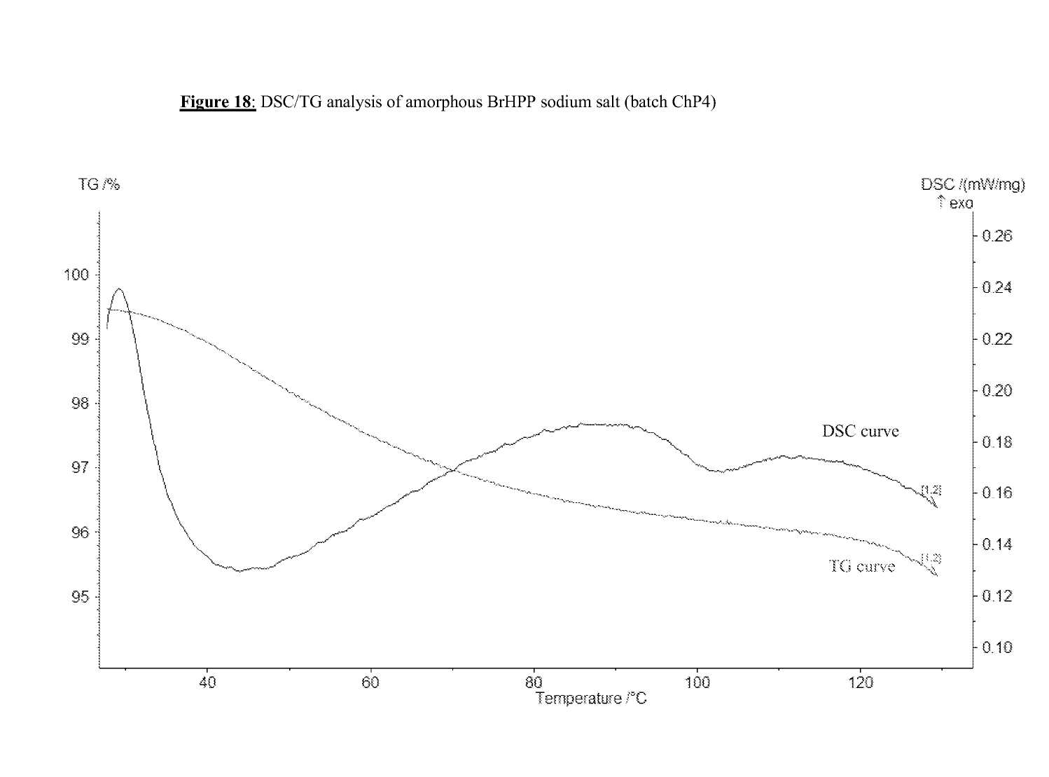

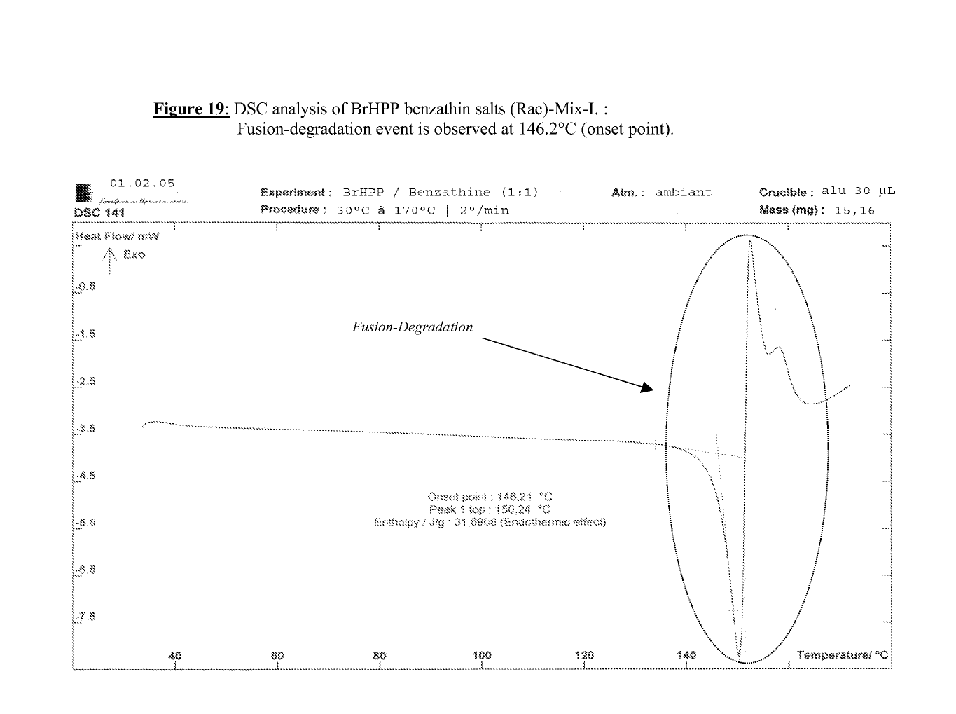

FIG. 18 shows the TG/DSC analysis of the BrHPP sodium salt (see TG/DSC procedure). The low but significant fluctuations of the DSC signal as well as the continuous mass loss are characteristic for an amorphous material that undergoes chemical degradations. This phenomenon occurs even for temperatures close to ambient.

By contrast, the DSC analysis (FIG. 19) of the (non solvated) crystalline form prepared according to example 4 (BrHPP benzathin salts, crystalline (Rac)-Mix-I)) reveals a high thermal stability profile since the fusion-degradation event only occurs above 146° C.

The stability of a sample of BrHPP Quinine salts, crystalline (n,p)-Mix-I which was stored in a glass vial at room temperature (20-25° C.) and ambient atmosphere (50-70% Relative Humidity) was evaluated using HPAEC analysis. The compound BrHPP was found to be stable over a period of at least 5 months. The anionic purity of this sample remained at a value of 99.5%±0.2% over that period.

### Example 10

### Hygroscopicity and Moisture Measurements

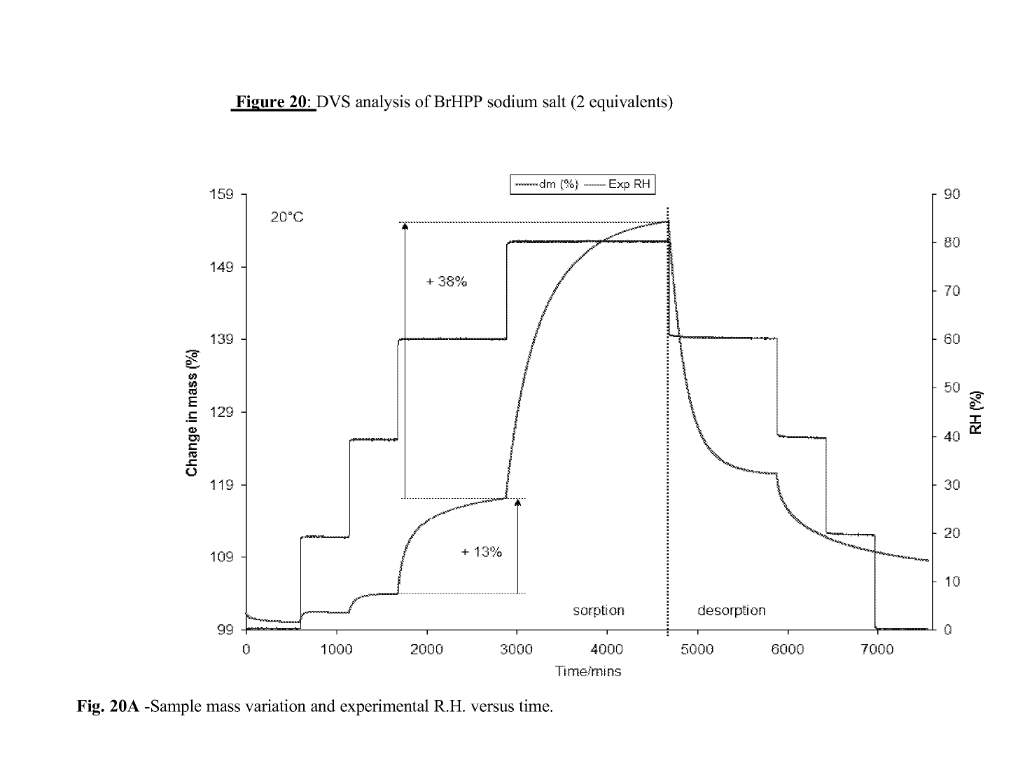

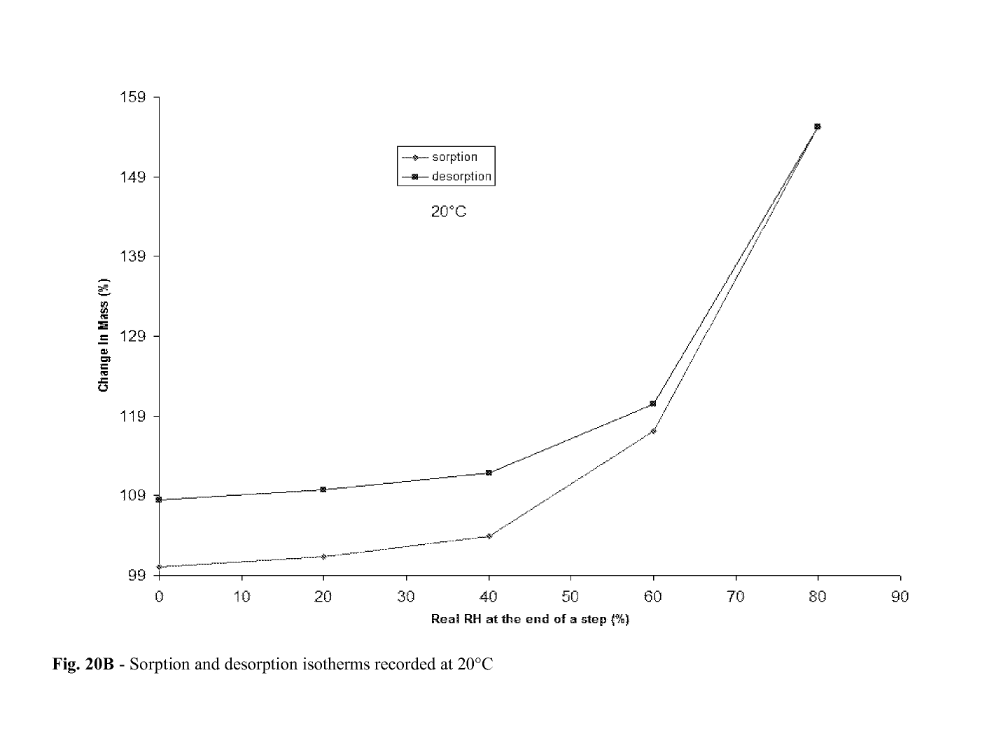

DVS measurements were performed with amorphous BrHPP disodium salt according to the first procedure. Sample mass variation and experimental R.H. are plotted as a function of time in FIG. 20-(a). Sorption and desorption isotherms are given in FIG. 20-(b). From 40% R.H. the water uptakes are high (3.8%<Δm<55.3%) and continuously increasing with R.H. For high relative humidities (≧80% R.H.), the stabilization of the mass could not be observed after 2300 minutes: at this stage, the compound becomes deliquescent (i.e. it absorbs vapour water molecules until dissolution, afterwards followed by dilution). Amorphous BrHPP sodium salt is very sensitive to atmospheric water, even for low R.H.

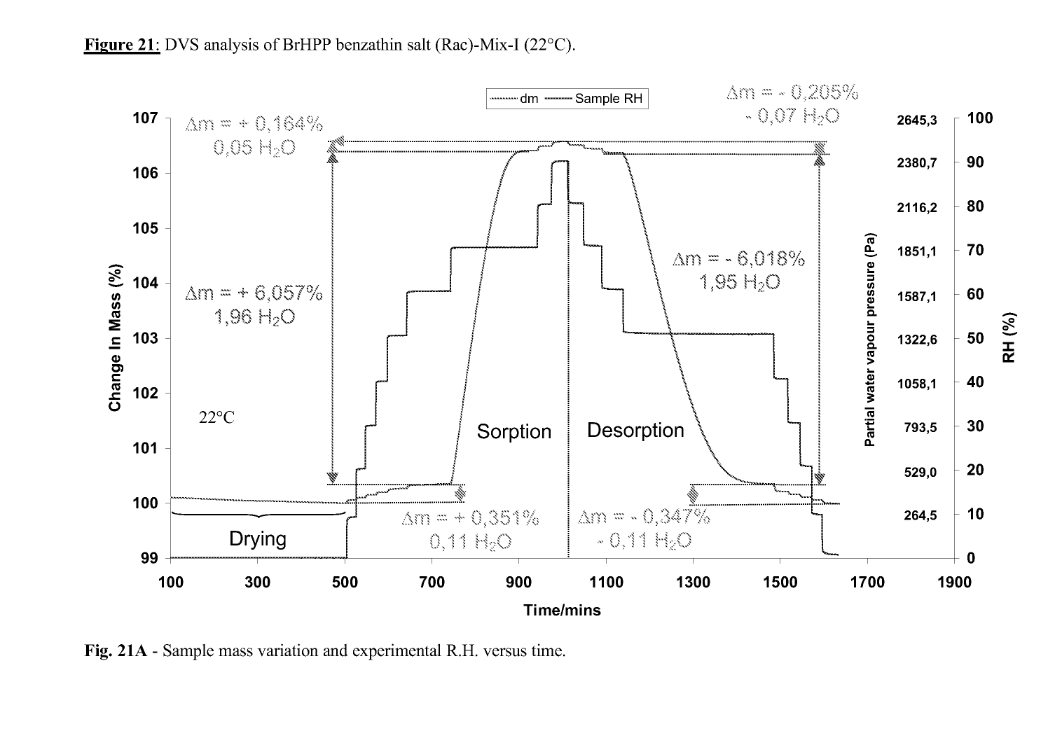

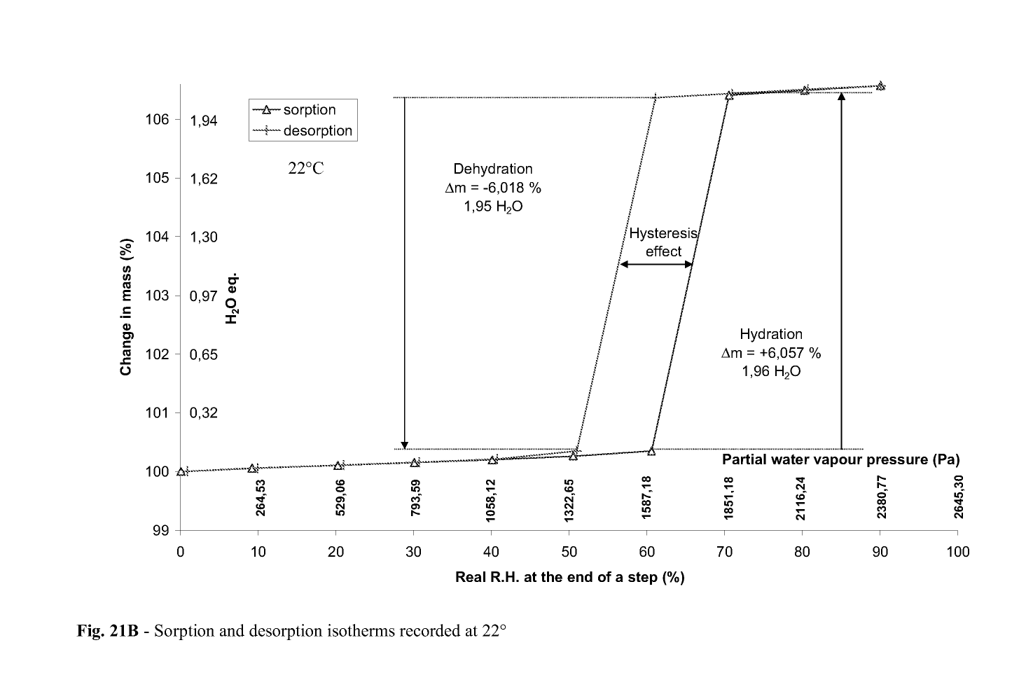

Comparatively, DVS analyses were also performed with BrHPP benzathin salt (Rac)-Mix-I according to the second procedure. Sample mass variations and experimental R.H. are plotted as a function of time in FIG. 21-(a). Sorption and desorption isotherms are reported in FIG. 21-(b). Between 0% and 50% R.H., mass variations are low (cumulated variations inferior to 0.4%) and probably associated with adsorption of water molecules on the crystal surfaces. For R.H. comprised in the range of 50%-70%, a reversible and reproducible mass variation of 6.1% is recorded. This observation is associated with the (de)hydration transformation between phase (Rac)-Mix-I (dehydrate) and (Rac)-Mix-III (dihydrate). The hysteresis could indicate even a metastability of the dehydrate above the threshold of hydration or a metastability of the dihydrate below the threshold of dehydration (FIG. 20-(b)). For high relative humidity (up to 90%), predominantly water adsorption phenomena occur as indicated by the low mass variations (cumulated variations inferior to 0.2%).

Crystalline BrHPP benzathin salts have been found to be stable for high relative humidity and no deliquescence phenomenon has been observed up to now. This behaviour ensures a great chemical stability of the title compound in time and also facilitates the storage operations (i.e. no particular need for dry atmosphere packaging).

### Example 11

### Preparation of (2Z)-4-hydroxy-2-methyl but-2-en-1-yl diphosphate, benzathine salt (H-angelyIPP Benzathin Salt)

### Preparation of H-AngelyIPP Benzathin Salt:

498 mg (1.440 mmoles, 1 equivalent) of ((2Z)-2-methyl-4-(tetrahydropyranyloxy)but-2-en-1-yl diphosphate, ammonium salt (THP protected H-angelyIPP, batch AB030, 60% purity based on HPAEC analysis) is diluted with 40 mL of deionized water at room temperature. The aqueous solution obtained is loaded on a column containing 126 milliequivalents (60 mL) of DOWEX 50WX8-200 cationic resin (H⁺ form), then eluted with 90 mL of deionized water (deprotection/acidification step—see note below). After elution, the volume of the resulting acidic solution of H-angelyIPP is ca. 130 mL. 40.0 mL of a 0.03 M ethanolic solution of the benzathin base (1.584 mmoles, 1.1 equivalent, 97% purity, purchased from SIGMA-ALDRICH®) is then added slowly while stirring to the acidic solution of H-angelylPP (salification step). The resulting mixture is a colourless solution. The entire amount of solvent is removed by evaporation under reduced pressure at 40° C. that allows to obtain 0.721 g of H-angelyIPP benzathin salt at the amorphous state (white solid). This residue is homogenized with 30 mL of Methanol while stirring at room temperature for 2 hours. The resulting suspension was filtered and the isolated solid phase (583 mg) was mixed with 15 mL oh Ethanol at room temperature. The temperature is then progressively decreased down to 10° C. followed by a maturation step (10 temperature cycles between 45° C. and 10° C. with a 15°/h cooling rate). The last cycle is stopped at 10° C. (during the cooling phase) and the solid phase is collected by filtration through a glass filter and allowed to dry at ambient atmosphere until constant weight. 360 mg of (2Z)-4-hydroxy-2-methylbut-2-en-1-yl diphosphate, benzathin salt (H-angelylPP benzathin salt) are obtained as a white solid by this procedure with a yield of =51% (95% purity based on HPAEC analysis). A “one pot” deprotection/acidification sequence is conducted in this example since the THP protective group of the allylic alcohol can be easily removed under acidic conditions.

1-96. (canceled)

97. A salt comprising the reaction product of a phosphoantigen and an organic base, wherein the phosphoantigen is a compound selected in the group consisting of compounds of Formulas I, II, IIa, III, IIIa, IIIa1, IIIa2, IIIa3, A, B, IIIb, IIIb1, IIIb2, IIIb3, C, IIIc, IIIc1, IIIc2, IIIc3, D, E, F and G.

98. The salt according to claim 97, wherein the phosphoantigen is substantially free of a different geometric isomer of the phosphoantigen.

99. The salt according to claim 97, wherein the organic base is selected from the group consisting of quinine, cinchonidine, cinchonine, quinidine, 8-hydroxy-quinoline, 5-chloro-8-hydroxy-quinoline, benzathin, procain, N-methyl-D-glucamine, diethylamine, ethylenediamine, piperazine, quinidine, quinoline, ethanolamine, thiethanolamine, betaine, natural alkaloid of quinquina, lysine and arginine and other basic and polar amino-acids.

100. The salt according to claim 97, wherein the phosphoantigen crystalline phase is characterised by an XRPD pattern comprising at least one, two, three, four or all of the peaks (2-theta angles) of the phases (A) to (O), said peaks of phases (A) to (O) being selected in the group consisting of the peaks at about:
(A) 7.52, 11.28, 16.75, 18.91 and 20.82;
(B) 7.71, 16.78 and 20.70;
(C) 7.66, 16.70 and 18.40;
(D) 6.65, 18.54 and 23.68;
(E) 5.96, 7.37, 16.06 and 19.27;
(F) 15.18, 15.51, 16.69, 17.78 and 26.24;
(G) 5.79, 11.46 and 17.14;
(H) 8.58, 17.13, 18.67 and 20.03;
(I) 8.38, 18.14, 19.71 and 19.96;
(J) 5.92 and 7.16;
(K) 5.53, 20.87, and 24.82;
(L) 8.33, 15.93, 18.06 and 19.89;
(M) 7.79, 17.51, 17.85 and 18.53;
(N) 5.62, 12.37, 16.41, 18.21, 18.70, 21.44 and 25.06; and
(O) 5.80, 8.68, 11.36, 23.32, 24.11.

101. The salt according to claim 97, wherein the phosphoantigen crystalline phase is characterised by an XRPD pattern comprising at least one of the peaks (2-theta angles) of the phases (A) to (O), said peaks of phases (A) to (O) being selected in the group consisting of the peaks at about:
(A) 5.64, 7.52, 11.28, 11.60, 12.92, 13.80, 15.71, 16.75, 17.49, 18.11, 18.44, 18.91, 19.25, 20.08, 20.82, 22.30, 23.96, 25.72, 26.56 and 27.24;
(B) 5.82, 7.71, 8.22, 15.55, 15.93, 16.45, 16.78, 17.60, 18.07, 18.68, 19.47 and 20.70;
(C) 5.81, 7.66, 16.70 and 18.40;
(D) 4.64, 6.65, 13.89, 14.24, 16.93, 18.54, 20.50, 23.68 and 27.90;
(E) 5.96, 7.37, 9.34, 9.70, 11.10, 12.38, 12.66, 13.69, 14.87, 16.06, 16.40, 18.20, 18.76, 19.27, 19.80, 20.86, 22.60, 23.00, 23.65, 24.32, 25.10, 25.50, 26.24, 26.62 and 27.03;
(F) 5.53, 10.69, 13.30, 13.97, 15.18, 15.51, 15.84, 16.69, 17.78, 18.12, 20.13, 20.67, 22.42, 23.85, 24.50, 25.42, 25.76, 26.24, 26.73 and 28.84;
(G) 5.79, 11.46, 16.19, 17.14, 17.39, 18.94 and 21.52;
(H) 8.58, 9.17, 10.07, 10.70, 14.33, 14.82, 16.04, 16.88, 17.13, 18.67, 20.03, 20.95, 22.42, 23.33, 25.34 and 25.64;
(I) 8.38, 15.74, 16.15, 18.14, 19.71, 19.96, 23.00, 25.06 and 25.99;
(J) 4.98, 5.92, 7.16 and 12.61;
(K) 5.53, 9.02, 10.61, 11.10, 14.31, 17.53, 19.83, 20.87, 23.49 and 24.82;
(L) 8.33, 13.56, 15.93, 16.74, 17.54, 18.06, 19.23, 19.89, 23.18, 24.98, 26.14 and 28.27;
(M) 6.83, 6.98, 7.79, 9.78, 13.71, 14.17, 14.41, 14.94, 15.38, 16.14, 17.28, 17.51, 17.85, 18.53, 18.77, 19.12, 19.50, 20.07, 20.93, 21.56, 21.73, 22.14, 24.09, 24.58, 24.96 and 25.68;
(N) 5.62, 10.26, 10.54, 11.22, 11.63, 12.37, 13.83, 14.88, 15.72, 16.41, 16.89, 17.12, 18.21, 18.70, 19.41, 20.63, 21.44, 21.85, 22.50, 23.31, 23.64, 24.11, 24.48, 25.06, 26.39, 27.14 and 29.62; and
(O) 5.80, 8.68, 10.58, 11.36, 11.60, 16.06, 16.70, 17.15, 17.45, 19.07, 19.52, 21.02, 21.82, 23.32, 24.11, 24.91, 25.33 and 28.16.

102. The salt according to claim 97, wherein said phosphoantigen salt has anionic purity of at least 95%.

103. The salt according to claim 97, wherein said phosphoantigen salt is stable for a period of time of at least 3 months at ambient temperature.

104. The salt according to claim 97, wherein said phosphoantigen salt decreases in anionic purity by less than 3% over said period of time.

105. The salt according to claim 97, wherein said phosphoantigen salt is substantially non-hygroscopic.

106. The salt according to claim 97, wherein the weight of said phosphoantigen salt does not vary by more than 5% at 80% relative humidity when the phosphoantigen is analyzed over a relative humidity range of from 0 to 90% in 3 steps and where each step is brought to equilibrium before moving to the next step, with equilibrium assessed as a weight change of less than 0.002 mg (0.02%) for five consecutive points at 1 point per 120 seconds.

107. The salt according to claim 97, wherein said phosphoantigen is 3-(bromomethyl)-3-butanol-1-yl-diphosphate (BrHPP), (E)-4-hydroxy-3-methyl-2-butenyl pyrophosphate (HDMAPP) or (E)-5-hydroxy-4-methylpent-3-enyl pyrophosphonate (CHDMAPP) made by reacting an organic base selected in the group consisting of quinine, cinchonidine, cinchonine, quinidine, 8-hydroxy-quinoline, 5-chloro-8-hydroxy-quinoline, benzathin, procain, N-methyl-D-glucamine, diethylamine, ethylenediamine, piperazine, ethanolamine, thiethanolamine, betaine, lysine, arginine and any other basic and polar amino-acid, wherein:
the form is stable for a period of time of at least 3 months at ambient temperature,
the form is substantially non-hygroscopic, or
the form has a purity of at least 99.5%.

108. The salt according to claim 97, wherein the phosphoantigen is a geometric isomer.

109. A composition comprising at least 10 g of a phosphoantigen salt according to claim 97.

110. A composition comprising a phosphoantigen salt according to claim 97 and a pharmaceutically acceptable carrier.

111. A method of preparing a crystalline phosphoantigen, the method comprising:
(a) acidification of a phosphoantigen,
(b) salification with an organic base, and
(c) isolating the crystals, thereby obtaining a crystalline phosphoantigen salt.

112. A method of purifying a phosphoantigen, the method comprising:
crystallizing a phosphoantigen according to the method of claim 111; and
recrystallizing the phosphoantigen to obtain a substantially pure phosphoantigen.

113. A method of preparing a crystalline phosphoantigen, the method comprising:
(a) acidification of the phosphoantigen;
(b) salification with an organic base solution;
(c) evaporation of solvent or distillation to form a solid;
(d) dissolution of the solid of step (c) in a minimal amount of a suitable solvent to form a suspension;
(e) anti-solvent addition, slow evaporation of the solvent in a controlled atmosphere or cooling of the suspension;
(f) maturation cycles on the suspension;
(g) isolation of a crystallized solid; and
(h) drying of the crystallized solid to obtain a crystalline phosphoantigen salt.

114. The method according to claim 113, further comprising washing or forming slurries of the crystallized solid.
